(19) United States
(12) Patent Application Publication
(10) Pub. No.: US 2023/0200227 A1
(43) Pub. Date: Jun. 22, 2023

(54) ORGANIC ELECTROLUMINESCENT DEVICE

(71) Applicant: BEIJING SUMMER SPROUT TECHNOLOGY CO., LTD., Beijing (CN)

(72) Inventors: Ziyan Zhang, Beijing (CN); Meiying Wang, Beijing (CN); Le Wang, Beijing (CN); Qiang Wang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 17/973,700

(22) Filed: Oct. 26, 2022

(30) Foreign Application Priority Data
Oct. 28, 2021 (CN) 202111253800.0

(51) Int. Cl. H01L 51/00; C09K 11/06

(52) U.S. Cl. CPC H01L 51/0067; C09K 11/06; H01L 51/0052; H01L 51/0072; H01L 51/0085; C09K 2211/185; C09K 2211/1029; H01L 51/5012

(57) ABSTRACT

Provided is an organic electroluminescent device. The organic electroluminescent device includes an anode, a cathode and an organic layer disposed between the anode and the cathode, where the organic layer at least comprises a first compound having a structure of H-L-E and a second compound having a structure of Formula 2. The first compound and the second compound may be used as host materials in the organic electroluminescent device. The electroluminescent device can have a longer device lifetime while maintaining a high level of current efficiency or further improving the current efficiency, and can provide better device performance. Further provided are an electronic apparatus and a compound combination.

### CROSS-REFERENCE TO RELATED APPLICATION(S)

This application claims priority to Chinese Patent Application No. 202111253800.0 filed on Oct. 28, 2021, the disclosure of which is incorporated herein by reference in its entirety.

### TECHNICAL FIELD

The present disclosure relates to an organic electronic device, for example, an organic electroluminescent device. More particularly, the present disclosure relates to an organic electroluminescent device comprising an organic layer which containing a first compound and a second compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

KR1020150077220A has disclosed a compound having the following structure:

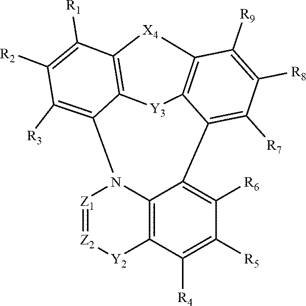

. However, this application does not disclose or teach use of the compound having the general formula mentioned above together with a indolocarbazole-based second host compound as host materials.

US20180337340A1 has disclosed a compound having the following structure:

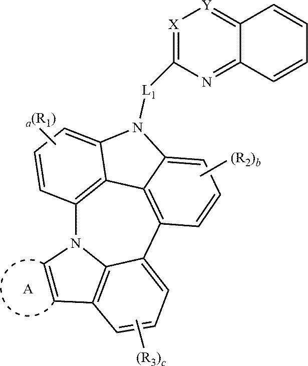

. The compound disclosed in this application must have a structure unit of quinazoline or quinoxaline. In addition, this application does not disclose or teach a compound combination comprising a compound that is formed by joining a carbazole-fused aza-seven-membered ring structure unit to triazine, or a similar structure, with an additional second host compound.

However, for many host materials reported at present, there is still room for improvement. To meet an increasing requirement of the industry, especially requirements for performance such as higher device efficiency, a longer device lifetime and a lower drive voltage, a new material combination still requires further research and development.

### SUMMARY

The present disclosure aims to provide a new electroluminescent device comprising an organic layer which containing a first compound and a second compound to solve at least part of the above problems. The first compound has a structure of H-L-E, and the second compound has a structure represented by Formula 2. The first compound and the second compound may be used as host materials in the electroluminescent device. The electroluminescent device can have a longer device lifetime while maintaining a high level of current efficiency or further improving the current efficiency, and can provide better device performance.

According to an embodiment of the present disclosure, disclosed is an electroluminescent device, which comprises an anode, a cathode and an organic layer disposed between the anode and the cathode, wherein the organic layer at least comprises a first compound and a second compound;
wherein the first compound has a structure of H-L-E, wherein H has a structure represented by Formula 1:

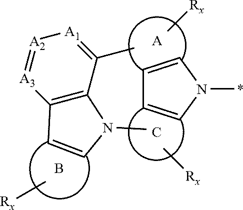

wherein in Formula 1, A₁, A₂ and A₃ are, at each occurrence identically or differently, selected from N or CR, and the ring A, the ring B and the ring C are, at each occurrence identically or differently, selected from a carbocyclic ring having 5 to 18 carbon atoms or a heterocyclic ring having 3 to 18 carbon atoms; and
Rₓ represents, at each occurrence identically or differently, mono-substitution, multiple substitutions or non-substitution;
wherein E has a structure represented by Formula 1-a:

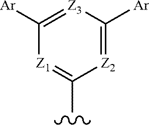

wherein in Formula 1-a, Ar is, at each occurrence identically or differently, selected from substituted or unsubstituted aryl having 6 to 30 carbon atoms or substituted or unsubstituted heteroaryl having 3 to 30 carbon atoms;
Z₁ to Z₃ are each independently selected from N or CR_{Z}, and at least one of Z₁ to Z₃ is N;
L is selected from a single bond, substituted or unsubstituted arylene having 6 to 30 carbon atoms, substituted or unsubstituted heteroarylene having 3 to 30 carbon atoms or a combination thereof;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R, Rₓ can be optionally joined to form a ring;
wherein the second compound has a structure represented by Formula 2:

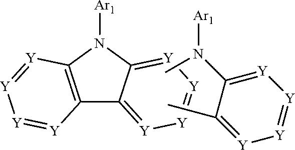

wherein
Y is, at each occurrence identically or differently, selected from C, CR_{Y} or N;
R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.

According to another embodiment of the present disclosure, further disclosed is an electronic apparatus, which comprises an electroluminescent device. A specific structure of the electroluminescent device is shown in any one of the preceding embodiments.

According to another embodiment of the present disclosure, further disclosed is a compound combination, which comprises a first compound and a second compound, wherein the first compound has a structure of H-L-E, wherein H has a structure represented by Formula 1:

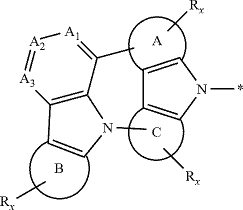

wherein in Formula 1, A₁, A₂ and A₃ are, at each occurrence identically or differently, selected from N or CR, and the ring A, the ring B and the ring C are, at each occurrence identically or differently, selected from a carbocyclic ring having 5 to 18 carbon atoms or a heterocyclic ring having 3 to 18 carbon atoms; and
Rₓ represents, at each occurrence identically or differently, mono-substitution, multiple substitutions or non-substitution;
wherein E has a structure represented by Formula 1-a:

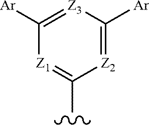

wherein in Formula 1-a, Ar is, at each occurrence identically or differently, selected from substituted or unsubstituted aryl having 6 to 30 carbon atoms or substituted or unsubstituted heteroaryl having 3 to 30 carbon atoms;
Z₁ to Z₃ are each independently selected from N or CR_{Z}, and at least one of Z₁ to Z₃ is N;
L is selected from a single bond, substituted or unsubstituted arylene having 6 to 30 carbon atoms, substituted or unsubstituted heteroarylene having 3 to 30 carbon atoms or a combination thereof;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R, Rₓ can be optionally joined to form a ring;
wherein the second compound has a structure represented by Formula 2:

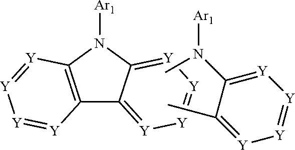

wherein
Y is, at each occurrence identically or differently, selected from C, CR_{Y} or N;
R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.

The new electroluminescent device disclosed in the present disclosure comprises an organic layer which containing the first compound and the second compound. The first compound and the second compound may be used as the host materials in the organic electroluminescent device. When the first compound and the second compound are used in combination, the electroluminescent device can have a longer device lifetime while maintaining a high level of current efficiency or further improving the current efficiency, and can provide better device performance.

### BRIEF DESCRIPTION OF DRAWINGS

FIG. 1 is a schematic diagram of an organic light-emitting apparatus that may comprise an electroluminescent device disclosed herein.

FIG. 2 is a schematic diagram of another organic light-emitting apparatus that may comprise an electroluminescent device disclosed herein.

### DETAILED DESCRIPTION

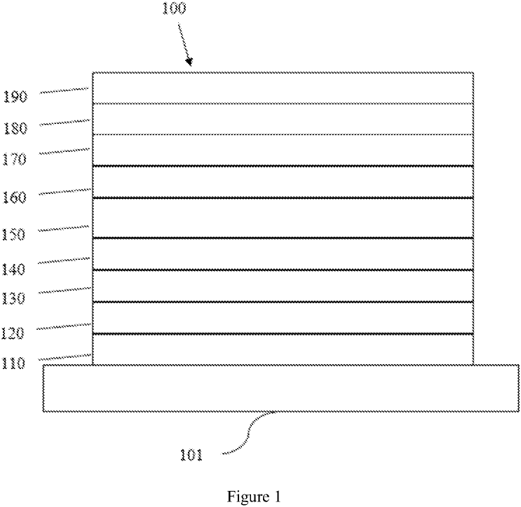

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Pat. Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Pat. Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Pat. Application Publication No. 2003/0230980, which are incorporated by reference herein in their entireties. Examples of injection layers are provided in U.S. Pat. Application Publication No. 2004/0174116, which is incorporated by reference herein in its entirety. A description of protective layers may be found in U.S. Pat. Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

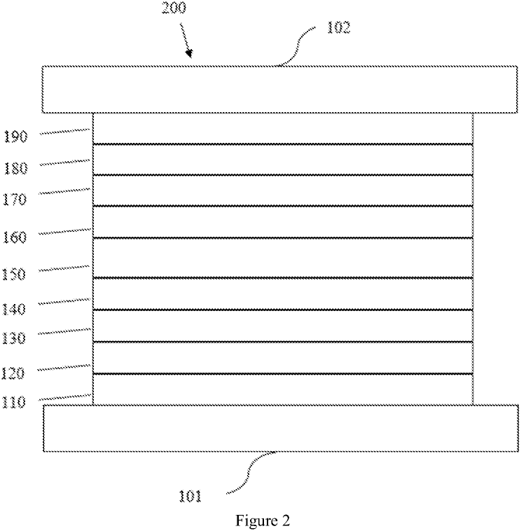

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small Δ_{ES-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide - as used herein includes fluorine, chlorine, bromine, and iodine.

Alkyl - as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl - as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl - as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted. Additionally, the heteroalkyl group may be optionally substituted.

Alkenyl - as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl - as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group - as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle - as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl - as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy - as used herein, is represented by —O—alkyl, —O—cycloalkyl, —O—heteroalkyl, or -O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy - as used herein, is represented by —O—aryl or —O—heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl - as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl - as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl - as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl - as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl - as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitution refers to a range that includes a di-substitution, up to the maximum available substitution. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

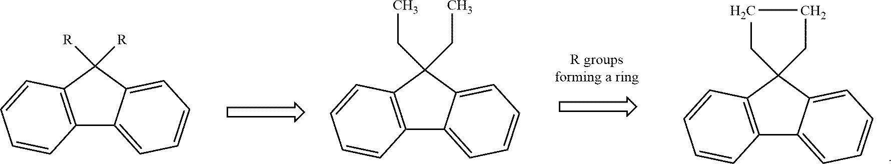

The expression that adjacent substituents can be optionally joined to form a ring is also intended to mean that two substituents bonded to carbon atoms which are directly bonded to each other are joined to each other via a chemical bond to form a ring, which can be exemplified by the following formula:

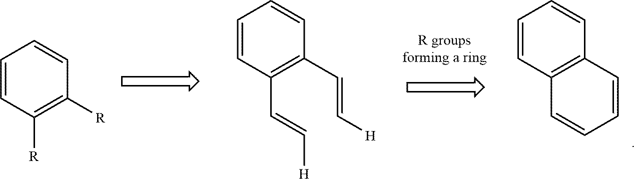

The expression that adjacent substituents can be optionally joined to form a ring is also intended to mean that two substituents bonded to a further distant carbon atom are joined to each other via a chemical bond to form a ring, which can be exemplified by the following formula:

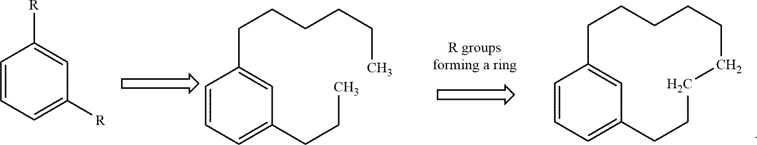

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

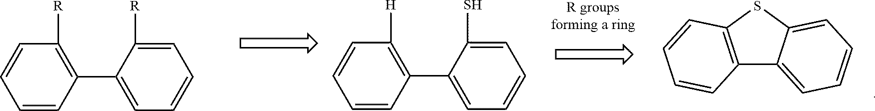

According to an embodiment of the present disclosure, disclosed is an electroluminescent device comprising:
an anode,
a cathode, and
an organic layer disposed between the anode and the cathode, wherein the organic layer at least comprises a first compound and a second compound;
wherein the first compound has a structure of H-L-E, wherein H has a structure represented by Formula 1:

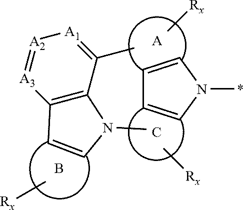

wherein in Formula 1, A₁, A₂ and A₃ are, at each occurrence identically or differently, selected from N or CR, and the ring A, the ring B and the ring C are, at each occurrence identically or differently, selected from a carbocyclic ring having 5 to 18 carbon atoms or a heterocyclic ring having 3 to 18 carbon atoms; and
Rₓ represents, at each occurrence identically or differently, mono-substitution, multiple substitutions or non-substitution;
wherein E has a structure represented by Formula 1-a:

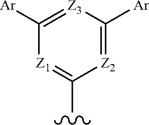

wherein in Formula 1-a, Ar is, at each occurrence identically or differently, selected from substituted or unsubstituted aryl having 6 to 30 carbon atoms or substituted or unsubstituted heteroaryl having 3 to 30 carbon atoms;
Z₁ to Z₃ are each independently selected from N or CR_{Z}, and at least one of Z₁ to Z₃ is N;
L is selected from a single bond, substituted or unsubstituted arylene having 6 to 30 carbon atoms, substituted or unsubstituted heteroarylene having 3 to 30 carbon atoms or a combination thereof;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R, Rₓ can be optionally joined to form a ring;
wherein the second compound has a structure represented by Formula 2:

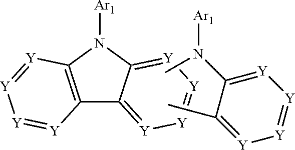

wherein Y is, at each occurrence identically or differently, selected from C, CR_{Y} or N;
R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.

In this embodiment, the expression that “adjacent substituents R, Rₓ can be optionally joined to form a ring” is intended to mean that any one or more of groups of adjacent substituents, such as adjacent substituents R, adjacent substituents Rₓ, and adjacent substituents R and Rₓ, can be joined to form a ring. Obviously, for those skilled in the art, it is possible that none of these groups of adjacent substituents are joined to form a ring.

In the present disclosure, the expression that “adjacent substituents R_{Y} can be optionally joined to form a ring” is intended to mean that any adjacent substituents R_{Y} can be joined to form a ring. Obviously, it is possible that any adjacent R_{Y} are not joined to form a ring.

According to an embodiment of the present disclosure, H has a structure represented by Formula 1A:

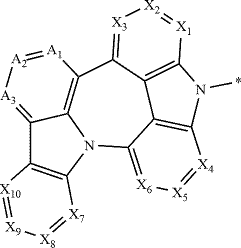

wherein A₁ to A₃ are, at each occurrence identically or differently, selected from N or CR, and X₁ to X₁₀ are, at each occurrence identically or differently, selected from N or CRₓ;
R and Rₓ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R, Rₓ can be optionally joined to form a ring.

In the present disclosure, the expression that “adjacent substituents R, Rₓ can be optionally joined to form a ring” is intended to mean that adjacent substituents R can be optionally joined to form a ring; also intended to mean that adjacent substituents Rₓ in X₁ to X₃ can be optionally joined to form a ring; also intended to mean that adjacent substituents Rₓ in X₄ to X₆ can be optionally joined to form a ring; also intended to mean that adjacent substituents Rₓ in X₇ to X₁₀ can be optionally joined to form a ring; and also intended to mean that adjacent substituents R and Rₓ can be optionally joined to form a ring. For example, adjacent substituents in A₁ and X₃, and/or A₃ and X₁₀, and/or X₆ and X₇ can be optionally joined to form a ring. Obviously, for those skilled in the art, adjacent substituents R and Rₓ may not be joined to form a ring. In this case, adjacent substituents R are not joined to form a ring, and/or adjacent substituents Rₓ are not joined to form a ring, and/or adjacent substituents R and Rₓ are also not joined to form a ring.

According to an embodiment of the present disclosure, R and Rₓ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted amino having 0 to 20 carbon atoms, a cyano group, an isocyano group, a hydroxyl group, a sulfanyl group and combinations thereof; and

adjacent substituents R and Rₓ can be optionally joined to form a ring.

According to an embodiment of the present disclosure, at least one of R and Rₓ is selected from deuterium, halogen, a cyano group, a hydroxyl group, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms or a combination thereof; and

adjacent substituents R and Rₓ can be optionally joined to form a ring.

According to an embodiment of the present disclosure, at least one of R and Rₓ is selected from deuterium, fluorine, a cyano group, a hydroxyl group, a sulfanyl group, methyl, trideuteromethyl, vinyl, phenyl, biphenyl, naphthyl, 4-cyanophenyl, dibenzofuranyl, dibenzothienyl, triphenylenyl, carbazolyl, 9-phenylcarbazolyl, 9,9-dimethylfluorenyl, pyridyl, phenylpyridyl or a combination thereof.

According to an embodiment of the present disclosure, H is selected from the group consisting of H-1 to H-139, wherein the specific structures of H-1 to H-139 are referred to claim 4.

According to an embodiment of the present disclosure, hydrogens in the structures of H-1 to H-139 can be partially or fully substituted with deuterium.

According to an embodiment of the present disclosure, E has a structure represented by Formula 1-a:

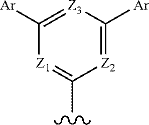

wherein Z₁ to Z₃ are each independently selected from N or CR_{Z}, and at least two of Z₁ to Z₃ are N;
R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
Ar is, at each occurrence identically or differently, selected from substituted or unsubstituted aryl having 6 to 18 carbon atoms or substituted or unsubstituted heteroaryl having 3 to 18 carbon atoms.

According to an embodiment of the present disclosure, Z₁ to Z₃ are all N.

According to an embodiment of the present disclosure, Ar is, at each occurrence identically or differently, selected from the group consisting of: phenyl, deuterated phenyl, methylphenyl, fluorophenyl, tert-butylphenyl, trideuteromethylphenyl, biphenyl, naphthyl, deuterated naphthyl, dibenzofuranyl, dibenzothienyl, 9,9-dimethylfluorenyl, carbazolyl, pyridyl, pyrimidinyl, 4-cyanophenyl, 3-cyanophenyl, triphenylenyl and combinations thereof.

According to an embodiment of the present disclosure, E is selected from the group consisting of E-1 to E-95, wherein the specific structures of E-1 to E-95 are referred to claim 6.

According to an embodiment of the present disclosure, hydrogens in the structures of E-1 to E-95 can be partially or fully substituted with deuterium.

According to an embodiment of the present disclosure, L is selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L has a structure represented by Formula 4:

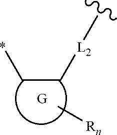

wherein the ring G is, at each occurrence identically or differently, selected from an aromatic ring having 6 to 18 carbon atoms or an heteroaromatic ring having 3 to 18 carbon atoms; L₂ is selected from a single bond, substituted or unsubstituted arylene having 6 to 30 carbon atoms, substituted or unsubstituted heteroarylene having 3 to 30 carbon atoms or a combination thereof; and when L₂ is selected from substituted arylene having 6 to 30 carbon atoms or substituted heteroarylene having 3 to 30 carbon atoms, L₂ has a substituent Rₘ, wherein Rₘ represents, at each occurrence identically or differently, mono-substitution or multiple substitutions; and
Rₙ and Rₘ are, at each occurrence identically or differently, selected from the group consisting of: hydrogen, deuterium, halogen, substituted or unsubstituted alkyl having 1 to 20 carbon atoms, substituted or unsubstituted arylalkyl having 7 to 30 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, an isocyano group, a hydroxyl group and combinations thereof;
adjacent substituents Rₙ, Rₘ can be optionally joined to form a ring.

In the present disclosure, the expression that “adjacent substituents Rₙ, Rₘ can be optionally joined to form a ring” is intended to mean that in the presence of substituents Rₙ and Rₘ, any one or more of groups of adjacent substituents, such as adjacent substituents Rₙ, adjacent substituents Rₘ, and substituents Rₙ and Rₘ, can be joined to form a ring. Obviously, in the presence of substituents Rₙ and Rₘ, it is possible that none of these groups of adjacent substituents are joined to form a ring.

According to an embodiment of the present disclosure, in Formula 4, the ring G is, at each occurrence identically or differently, selected from an aromatic ring having 6 to 12 carbon atoms or a heteroaromatic ring having 3 to 12 carbon atoms.

According to an embodiment of the present disclosure, in Formula 4, L₂ is selected from a single bond, substituted or unsubstituted arylene having 6 to 12 carbon atoms, substituted or unsubstituted heteroarylene having 3 to 12 carbon atoms or a combination thereof.

According to an embodiment of the present disclosure, in Formula 4, the ring G is, at each occurrence identically or differently, selected from a benzene ring, a naphthalene ring, a phenanthrene ring, a fluorene ring, a triphenylene ring, a carbazole ring, a dibenzofuran ring, a dibenzothiophene ring, a pyridine ring or a combination thereof;
L₂ is selected from a single bond, substituted or unsubstituted phenylene or substituted or unsubstituted naphthylene; and
Rₙ and Rₘ are, at each occurrence identically or differently, selected from the group consisting of: hydrogen, deuterium, halogen, substituted or unsubstituted alkyl having 1 to 20 carbon atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted amino having 0 to 20 carbon atoms, a cyano group, a hydroxyl group, a sulfanyl group and combinations thereof.

According to an embodiment of the present disclosure, L is selected from the group consisting of the following structures:

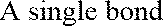

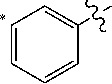

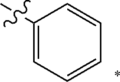

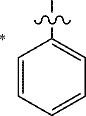

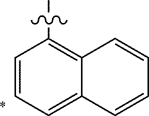

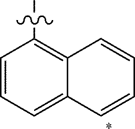

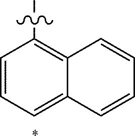

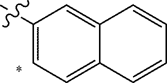

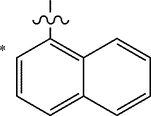

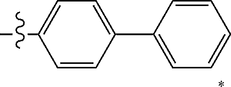

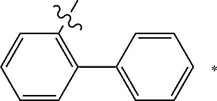

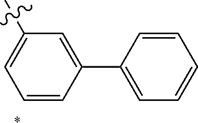

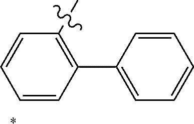

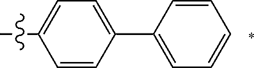

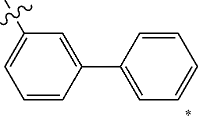

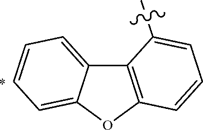

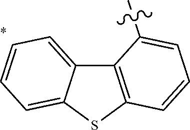

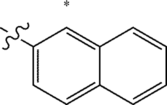

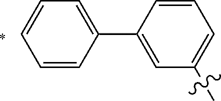

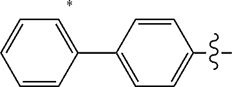

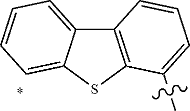

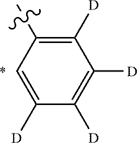

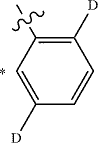

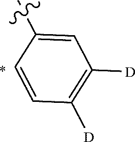

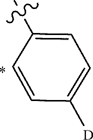

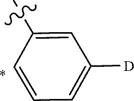

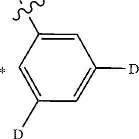

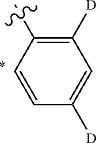

wherein “

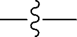

” represents a position where the structure of L-1 to L-27 is joined to E, and “*” represents a position where the structure of L-1 to L-27 is joined to H.

According to an embodiment of the present disclosure, hydrogens in the structures of L-1 to L-27 can be partially or fully substituted with deuterium.

According to an embodiment of the present disclosure, the first compound has the structure of H-L-E, wherein H is selected from any one of the group consisting of H-1 to H-139, L is selected from any one of the group consisting of L-0 to L-27, and E is selected from any one of the group consisting of E-1 to E-95; optionally, hydrogens in the first compound can be partially or fully substituted with deuterium.

According to an embodiment of the present disclosure, the first compound is selected from the group consisting of Compound 1-1 to Compound 1-550, wherein the specific structures of Compound 1-1 to Compound 1-550 are referred to claim 8.

According to an embodiment of the present disclosure, hydrogens in Compound 1-1 to Compound 1-550 can be partially or fully substituted with deuterium.

According to an embodiment of the present disclosure, in Formula 2, Y is, at each occurrence identically or differently, selected from C or CR_{Y}.

According to an embodiment of the present disclosure, the second compound has a structure represented by one of Formulas 2-a to 2-d:

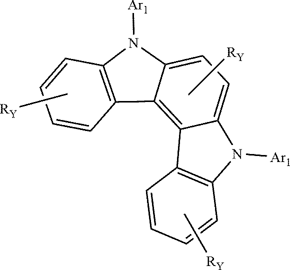

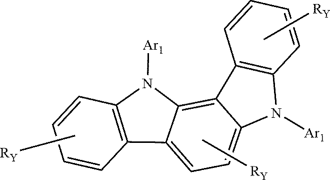

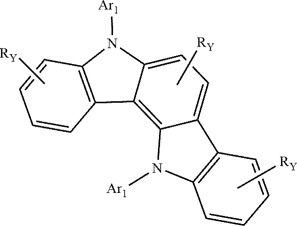

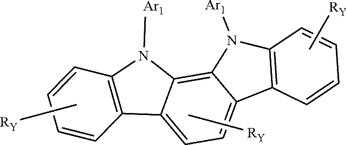

wherein R_{Y} represents, at each occurrence identically or differently, mono-substitution, multiple substitutions or non-substitution; R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.

According to an embodiment of the present disclosure, R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, a substituted or unsubstituted heterocyclic group having 3 to 20 ring atoms, substituted or unsubstituted aryl having 6 to 20 carbon atoms, substituted or unsubstituted heteroaryl having 3 to 20 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a cyano group, an isocyano group, a sulfanyl group, a sulfinyl group, a sulfonyl group, a phosphino group and combinations thereof.

According to an embodiment of the present disclosure, Ar₁ is, at each occurrence identically or differently, selected from substituted or unsubstituted aryl having 6 to 25 carbon atoms, substituted or unsubstituted heteroaryl having 3 to 25 carbon atoms or a combination thereof.

According to an embodiment of the present disclosure, Ar₁ is, at each occurrence identically or differently, selected from phenyl, fluorophenyl, naphthyl, biphenyl, benzothienyl, dibenzothienyl, benzofuranyl, dibenzofuranyl, dibenzoselenophenyl, carbazolyl, 9,9-dimethylfluorenyl, 9,9-spirobifluorenyl, cyanophenyl, phenanthryl, acridinyl, benzoacridinyl, adamantane spirofluorenyl or a combination thereof.

According to an embodiment of the present disclosure, Ar₁ is selected from the group consisting of Ar-1 to Ar-132, wherein the specific structures of Ar-1 to Ar-132 are referred to claim 10.

According to an embodiment of the present disclosure, hydrogens in the structures of Ar-1 to Ar-132 can be partially or fully substituted with deuterium.

According to an embodiment of the present disclosure, the second compound is selected from the group consisting of Compound 2-1 to Compound 2-305, wherein the specific structures of Compound 2-1 to Compound 2-305 are referred to claim 11.

According to an embodiment of the present disclosure, hydrogens in Compound 2-1 to Compound 2-305 can be partially or fully substituted with deuterium.

According to an embodiment of the present disclosure, the organic layer is a light-emitting layer, and the first compound and the second compound are host materials.

According to an embodiment of the present disclosure, the light-emitting layer further comprises at least one phosphorescent material.

According to an embodiment of the present disclosure, the at least one phosphorescent material is a metal complex having a general formula of M(Lₐ)ₘ(L_{b})ₙ(L_{c})_{q}; M is selected from a metal with a relative atomic mass greater than 40; and Lₐ, L_{b} and L_{c} are a first ligand, a second ligand and a third ligand coordinated to M, respectively; Lₐ, L_{b} and L_{c} can be optionally joined to form a multidentate ligand; Lₐ, L_{b} and L_{c} may be identical or different; m is 1, 2 or 3; n is 0, 1 or 2; q is 0, 1 or 2; the sum of m, n and q is equal to an oxidation state of M; when m is greater than or equal to 2, a plurality of Lₐ may be identical or different; when n is 2, two L_{b} may be identical or different; when q is 2, two L_{c} may be identical or different;
wherein Lₐ has a structure represented by Formula 3:

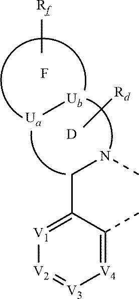

wherein
the ring D is selected from a five-membered heteroaromatic ring or a six-membered heteroaromatic ring;
the ring F is selected from a five-membered unsaturated carbocyclic ring, a benzene ring, a five-membered heteroaromatic ring or a six-membered heteroaromatic ring;
the ring D and the ring F are fused via Uₐ and U_{b};
Uₐ and U_{b} are, at each occurrence identically or differently, selected from C or N;
R_{d} and R_{f} represent, at each occurrence identically or differently, mono-substitution, multiple substitutions or non-substitution;
V₁ to V₄ are, at each occurrence identically or differently, selected from CRᵥ or N;
R_{d}, R_{f} and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{d}, R_{f}, Rᵥ can be optionally joined to form a ring;
wherein L_{b} and L_{c} are, at each occurrence identically or differently, selected from any one of the following structures:

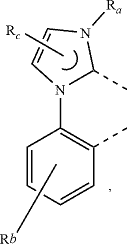

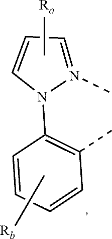

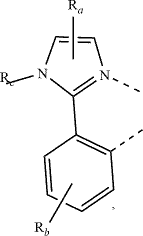

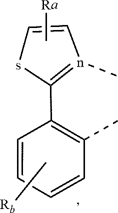

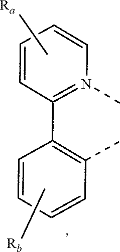

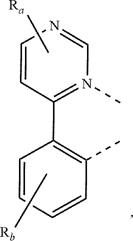

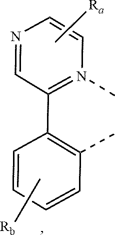

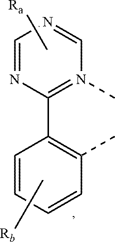

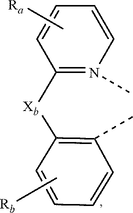

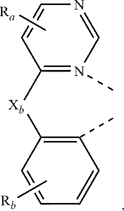

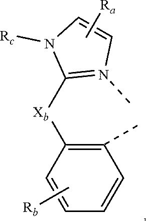

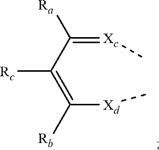

wherein Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
in the structures of the ligands L_{b} and L_{c}, adjacent substituents Rₐ, R_{b}, R_{c}, R_{N1}, R_{N2}, R_{C1} and R_{C2} can be optionally joined to form a ring.

In the present disclosure, the expression that “adjacent substituents R_{d}, R_{f}, Rᵥ can be optionally joined to form a ring” is intended to mean that any one or more of groups of adjacent substituents, such as adjacent substituents R_{d}, adjacent substituents R_{f}, adjacent substituents Rᵥ, adjacent substituents R_{d} and Rᵥ, adjacent substituents R_{f} and Rᵥ, and adjacent substituents R_{d} and R_{f}, can be joined to form a ring. Obviously, for those skilled in the art, it is possible that none of these groups of adjacent substituents are joined to form a ring.

In this embodiment, the expression that “adjacent substituents Rₐ, R_{b}, R_{c}, R_{N1}, R_{N2}, R_{C1} and R_{C2} can be optionally joined to form a ring” is intended to mean that any one or more of groups of adjacent substituents, such as adjacent substituents Rₐ, adjacent substituents R_{b}, adjacent substituents R_{c}, adjacent substituents Rₐ and R_{b}, adjacent substituents Rₐ and R_{c}, adjacent substituents R_{b} and R_{c}, adjacent substituents Rₐ and R_{N1}, adjacent substituents R_{b} and R_{N1}, adjacent substituents Rₐ and R_{C1}, adjacent substituents Rₐ and R_{C2}, adjacent substituents R_{b} and R_{C1}, adjacent substituents R_{b} and R_{C2}, adjacent substituents Rₐ and R_{N2}, and adjacent substituents R_{b} and R_{N2}, can be joined to form a ring. Obviously, it is possible that none of these substituents are joined to form a ring.

According to an embodiment of the present disclosure, the at least one phosphorescent material is a metal complex having a general formula of M(Lₐ)ₘ(L_{b})ₙ;
M is selected from a metal with a relative atomic mass greater than 40;
Lₐ and L_{b} are a first ligand and a second ligand coordinated to M, respectively; Lₐ and L_{b} can be optionally joined to form a multidentate ligand; and
m is 1, 2 or 3; n is 0, 1 or 2; the sum of m and n is equal to an oxidation state of M; when m is greater than or equal to 2, a plurality of Lₐ may be identical or different; when n is 2, two L_{b} may be identical or different;
wherein Lₐ has a structure represented by Formula 3:

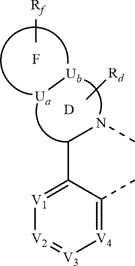

wherein
the ring D is selected from a five-membered heteroaromatic ring or a six-membered heteroaromatic ring;
the ring F is selected from a five-membered unsaturated carbocyclic ring, a benzene ring, a five-membered heteroaromatic ring or a six-membered heteroaromatic ring;
the ring D and the ring F are fused via Uₐ and U_{b};
Uₐ and U_{b} are, at each occurrence identically or differently, selected from C or N;
R_{d} and R_{f} represent, at each occurrence identically or differently, mono-substitution, multiple substitutions or non-substitution;
V₁ to V₄ are, at each occurrence identically or differently, selected from CRᵥ or N;
R_{d}, R_{f} and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{d}, R_{f}, Rᵥ can be optionally joined to form a ring;
wherein the ligand L_{b} has the following structure:

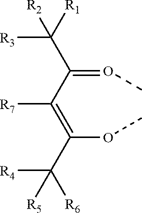

wherein R₁ to R₇ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof.

According to an embodiment of the present disclosure, at least one or two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one or two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof; and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

According to an embodiment of the present disclosure, in the device, the at least one phosphorescent material is an Ir complex, a Pt complex or an Os complex.

According to an embodiment of the present disclosure, in the device, the at least one phosphorescent material is an Ir complex and has a structure represented by any one of Ir(Lₐ)(L_{b})(L_{c}), Ir(Lₐ)₂(L_{b}), Ir(Lₐ)₂(L_{c}) or Ir(Lₐ)(L_{c})₂.

According to an embodiment of the present disclosure, in the electroluminescent device, the at least one phosphorescent material is an Ir complex and comprises a ligand Lₐ, wherein the Lₐ has the structure represented by Formula 3 and comprises at least one structure unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring and a heteroaromatic ring formed by fusing a six-membered ring to a five-membered ring.

According to an embodiment of the present disclosure, in the electroluminescent device, the at least one phosphorescent material is an Ir complex and comprises a ligand Lₐ, wherein the Lₐ has the structure represented by Formula 3 and comprises at least one structure unit selected from the group consisting of naphthalene, phenanthrene, quinoline, isoquinoline and azaphenanthrene.

According to an embodiment of the present disclosure, in the electroluminescent device, the at least one phosphorescent material is an Ir complex and comprises a ligand Lₐ, wherein the Lₐ is, at each occurrence, selected from any one of the group consisting of the following structures:

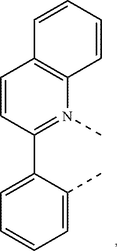

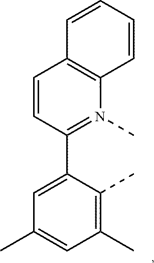

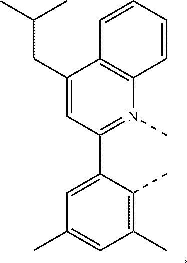

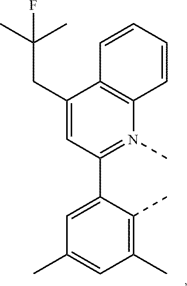

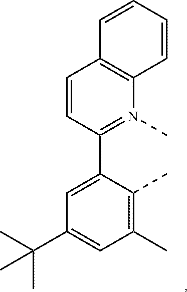

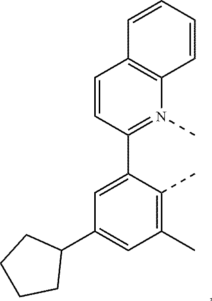

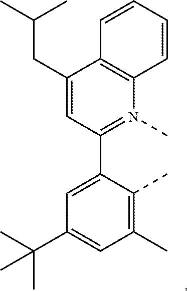

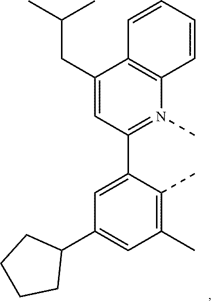

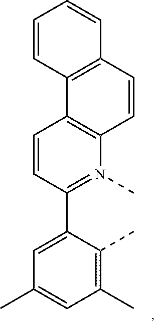

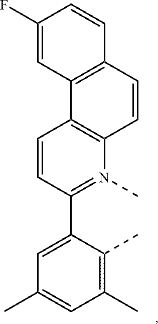

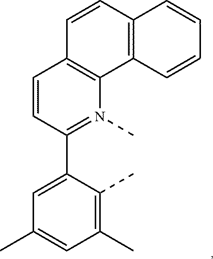

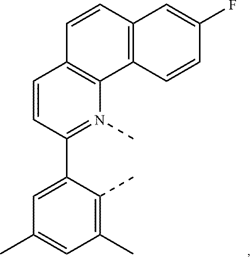

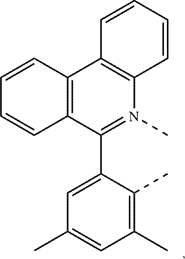

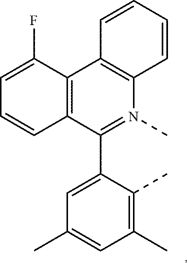

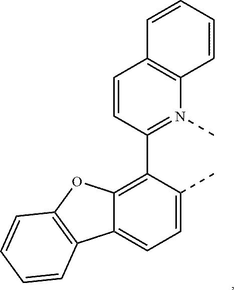

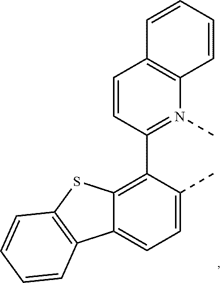

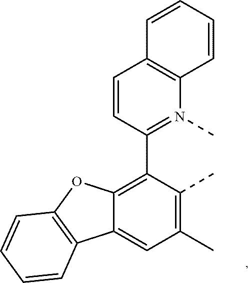

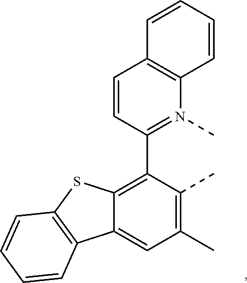

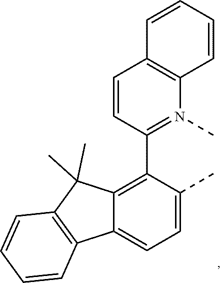

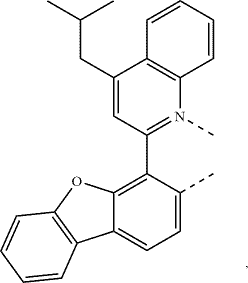

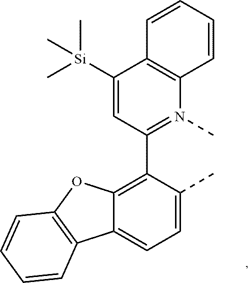

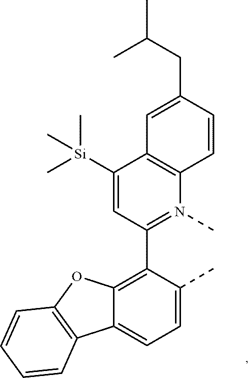

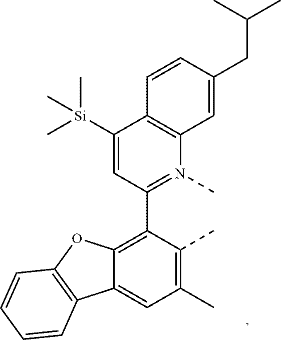

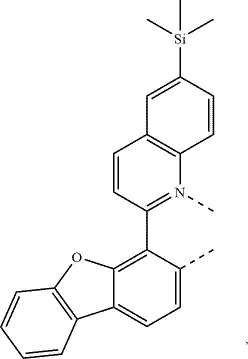

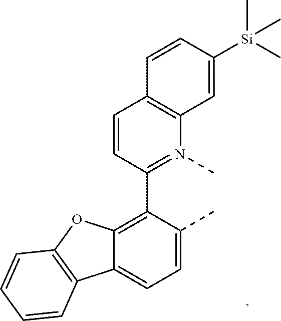

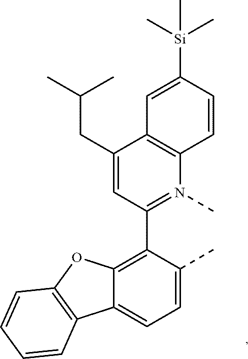

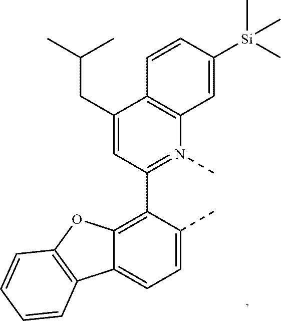

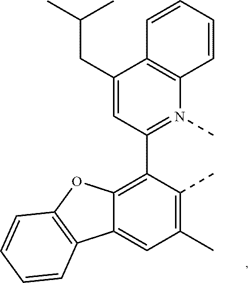

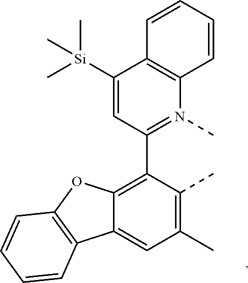

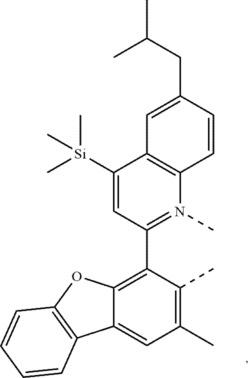

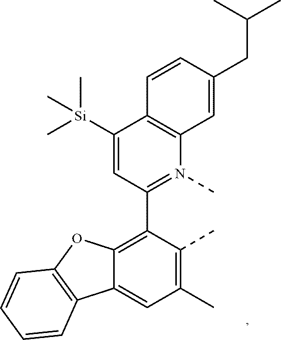

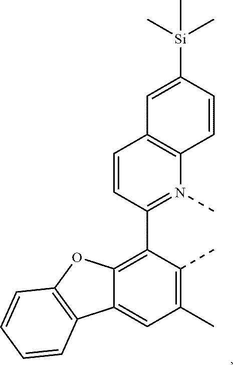

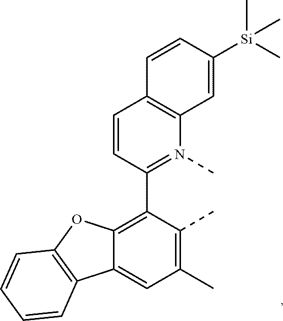

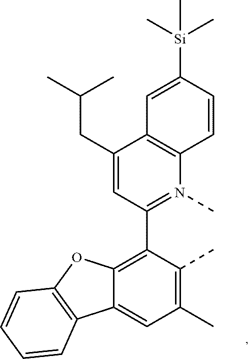

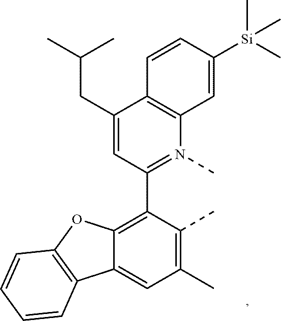

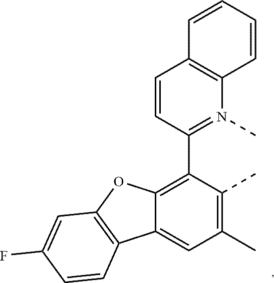

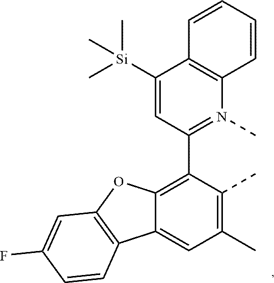

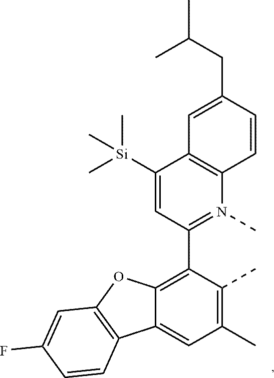

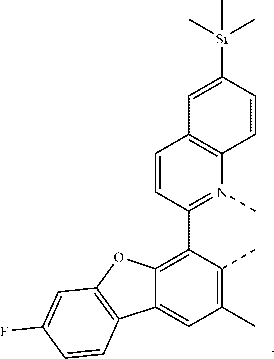

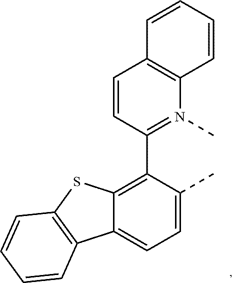

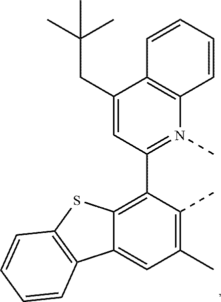

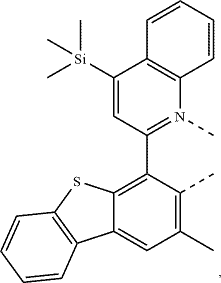

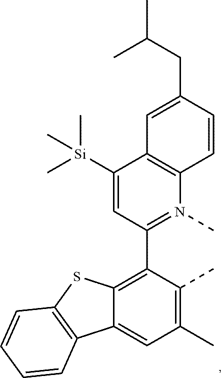

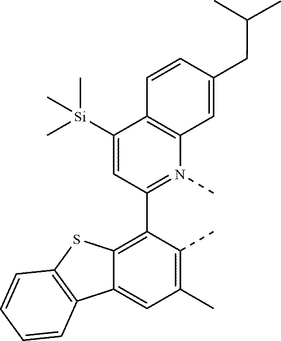

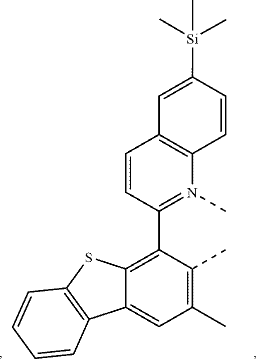

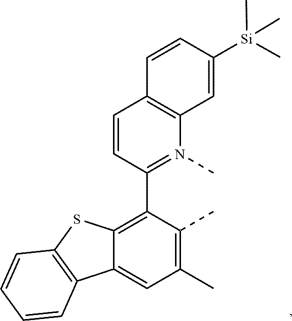

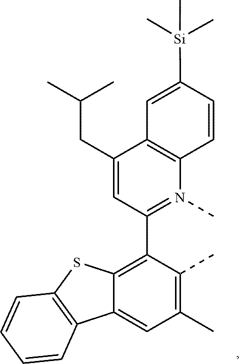

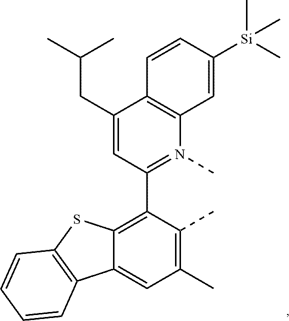

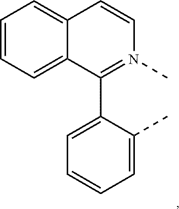

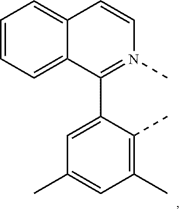

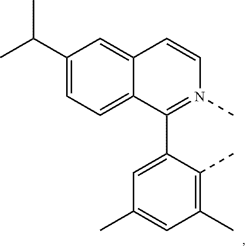

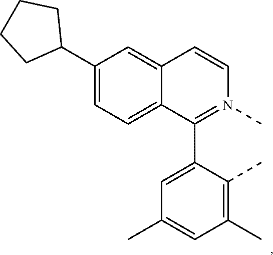

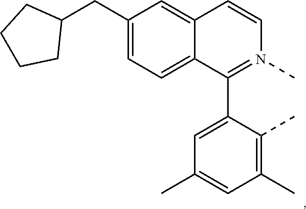

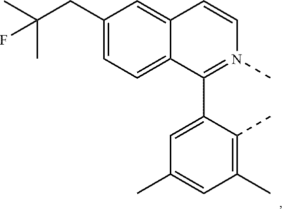

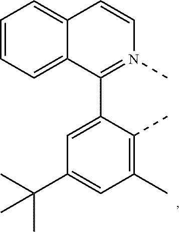

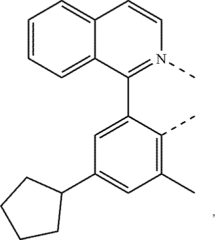

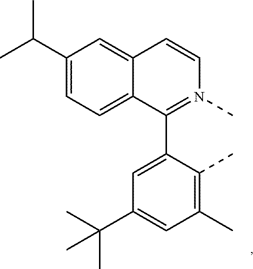

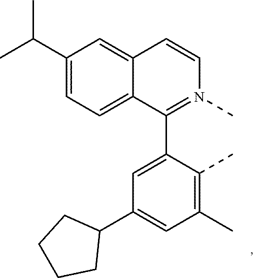

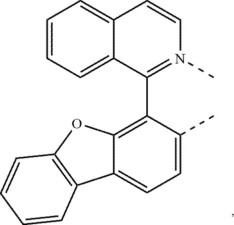

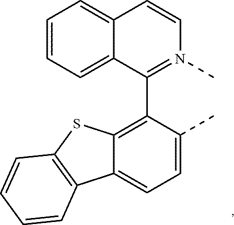

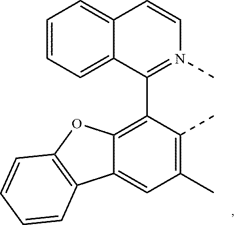

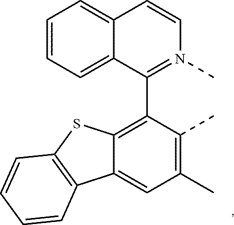

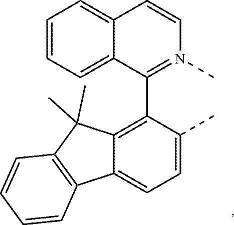

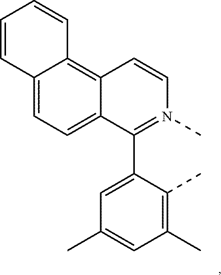

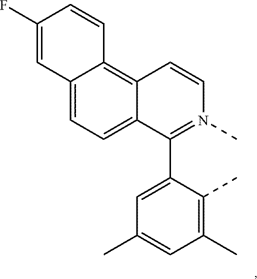

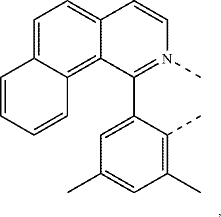

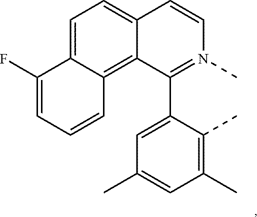

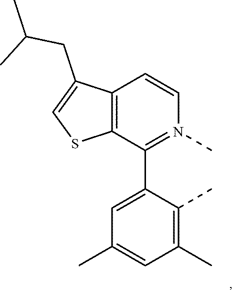

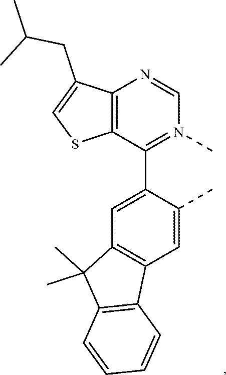

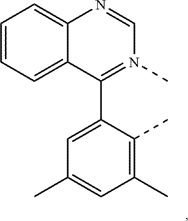

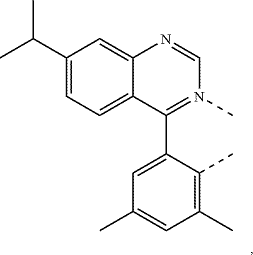

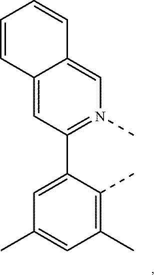

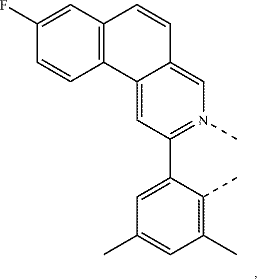

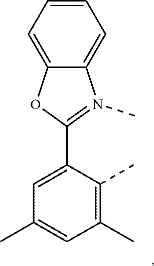

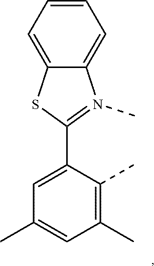

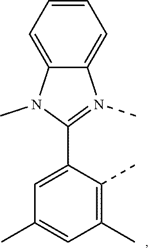

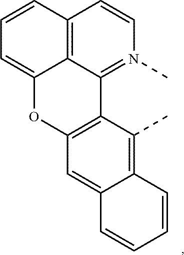

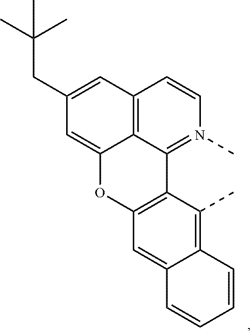

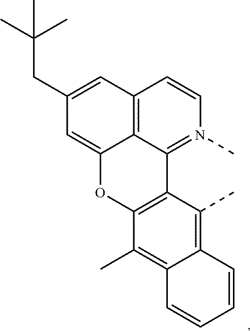

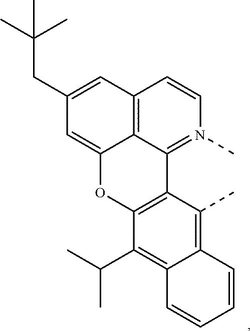

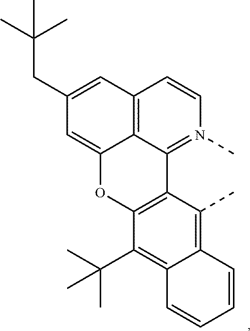

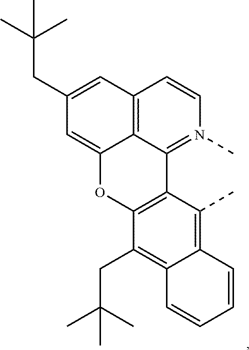

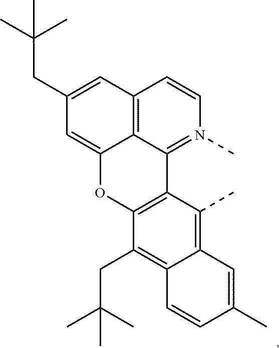

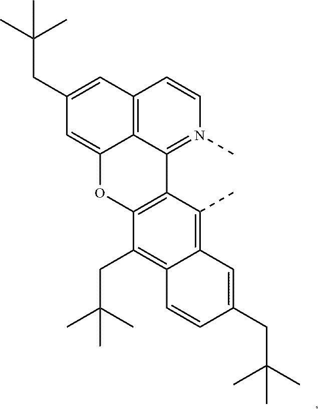

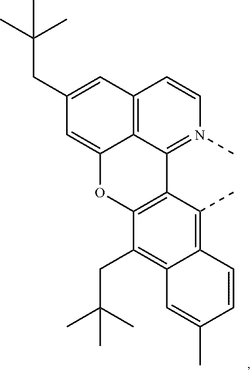

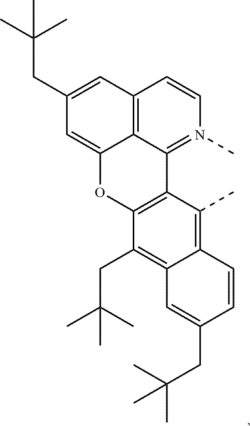

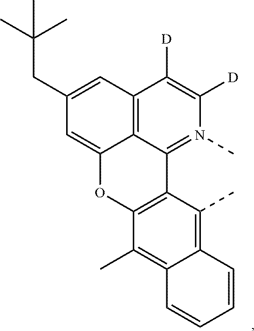

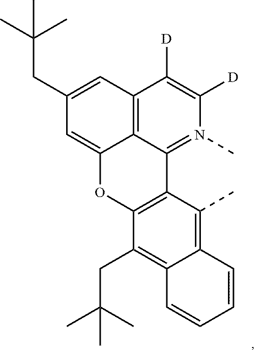

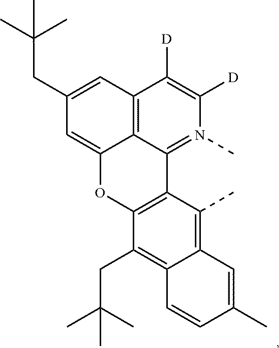

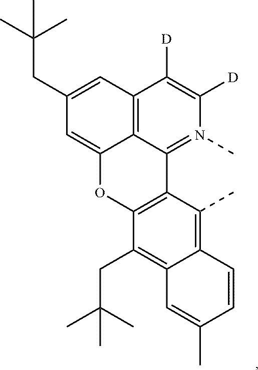

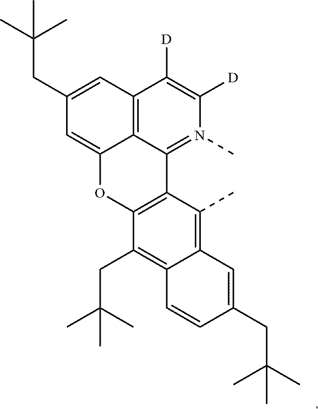

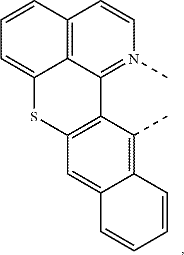

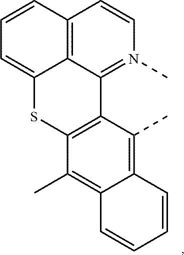

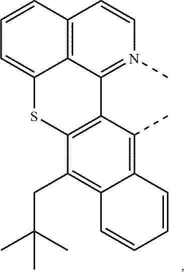

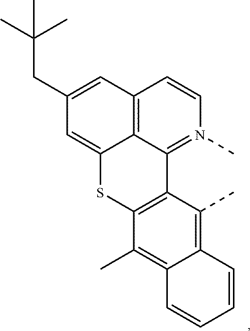

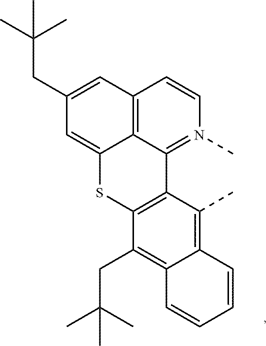

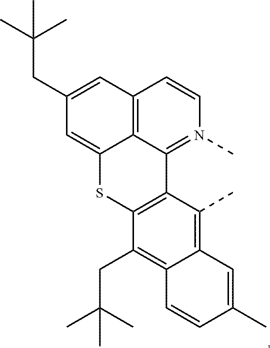

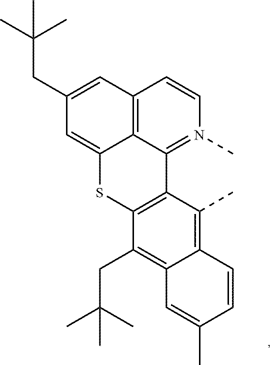

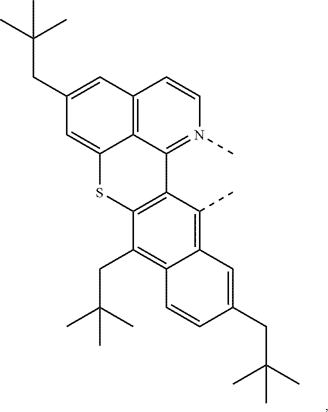

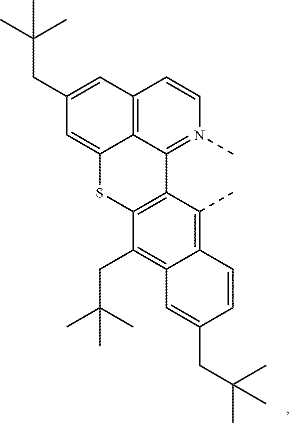

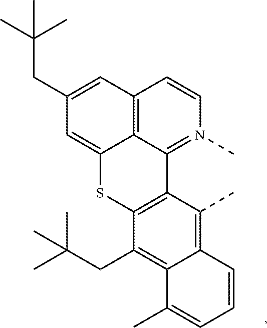

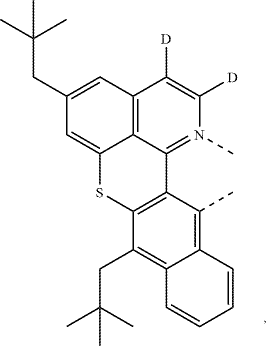

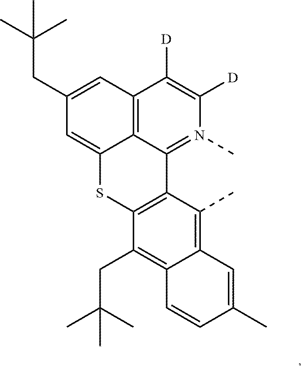

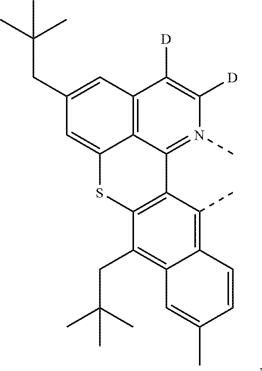

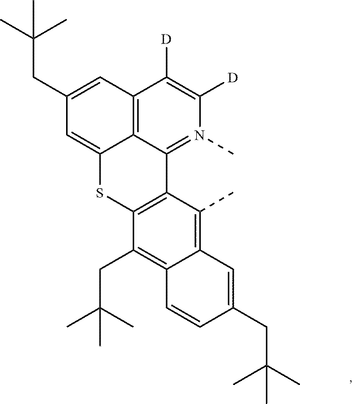

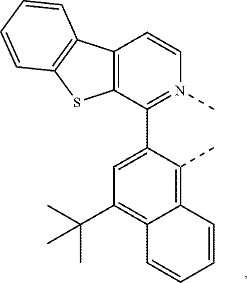

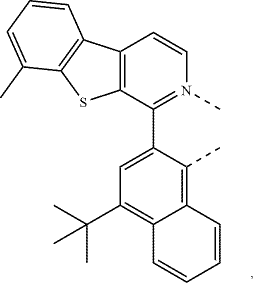

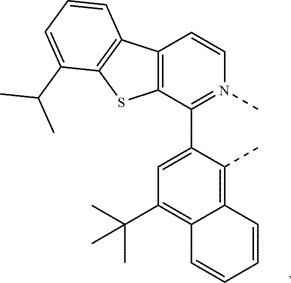

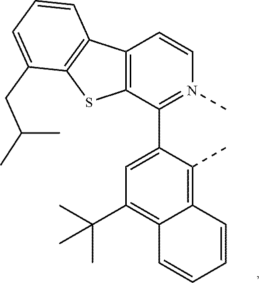

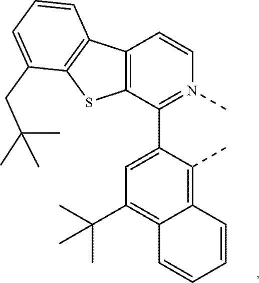

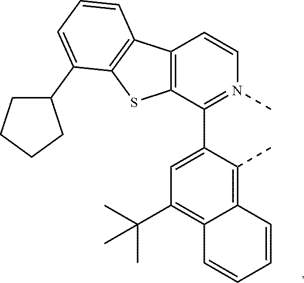

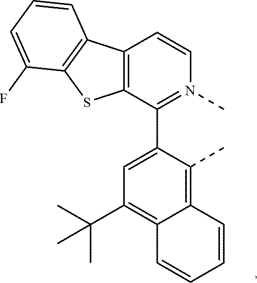

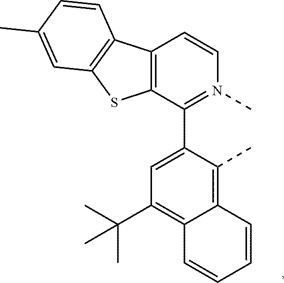

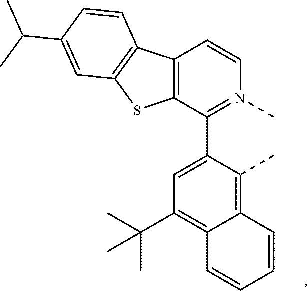

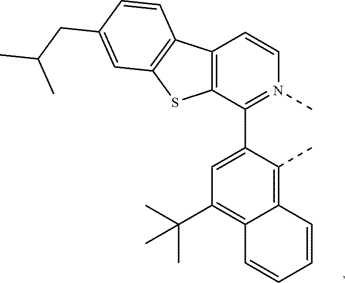

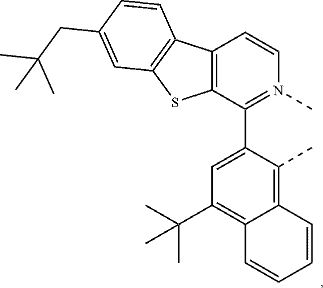

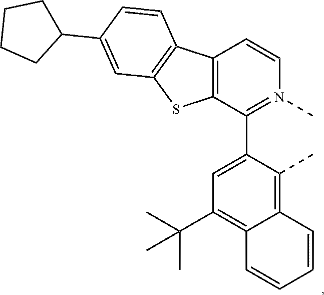

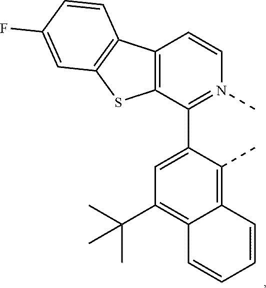

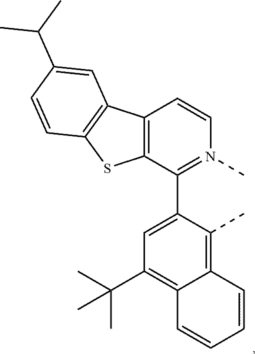

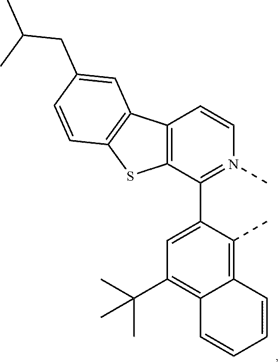

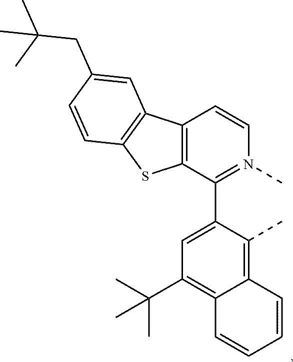

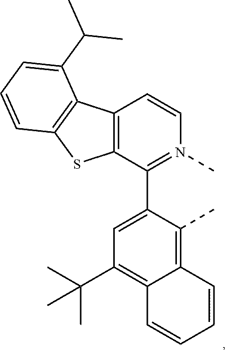

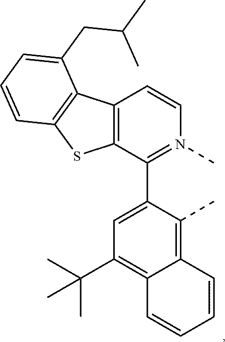

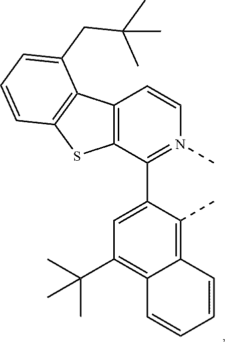

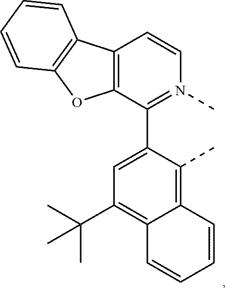

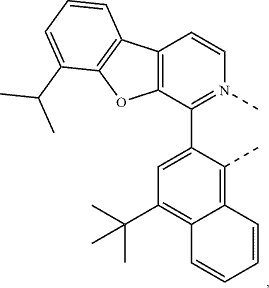

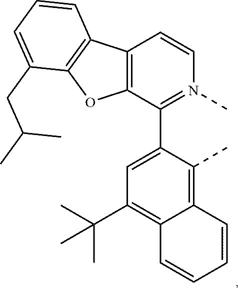

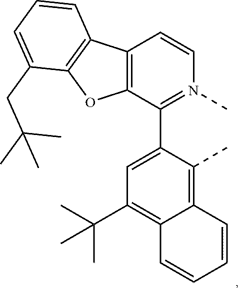

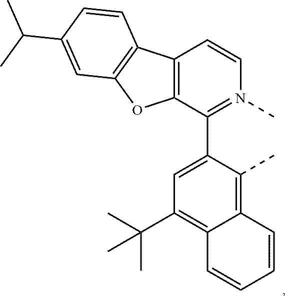

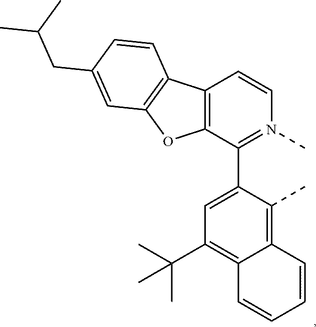

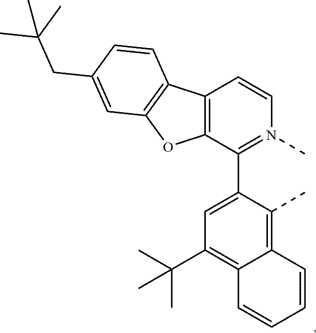

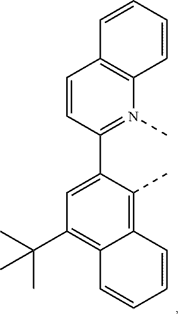

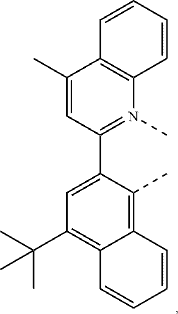

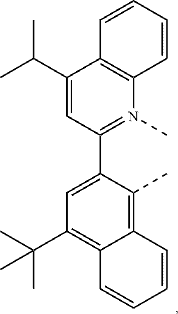

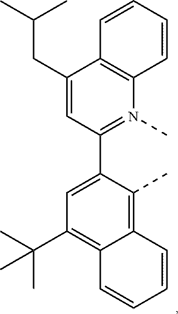

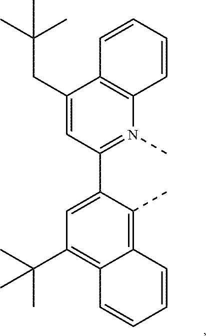

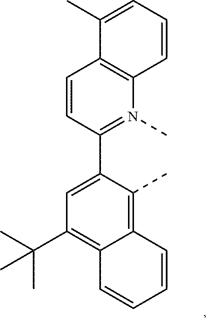

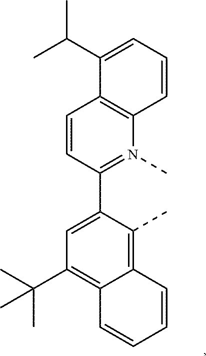

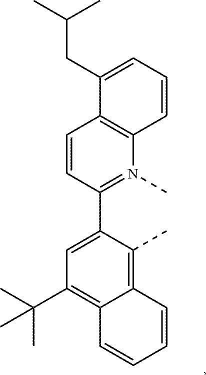

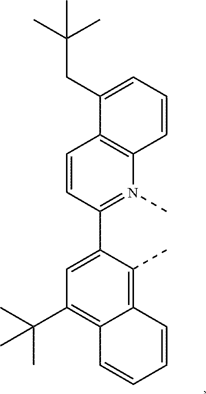

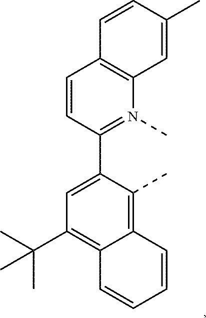

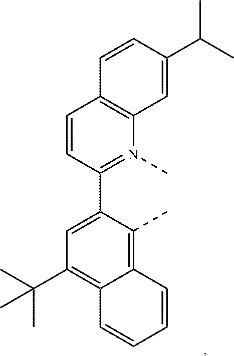

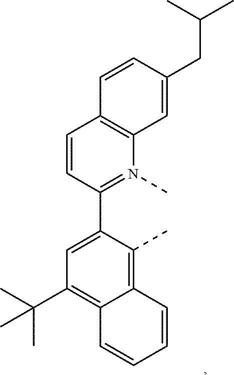

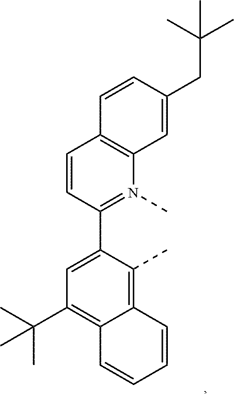

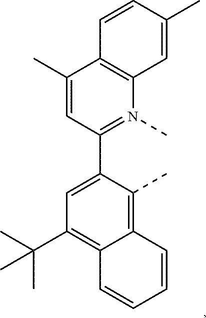

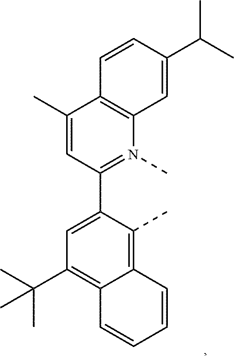

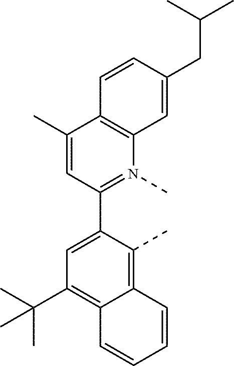

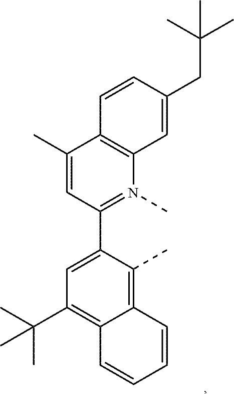

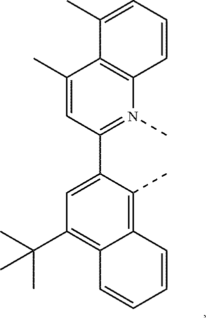

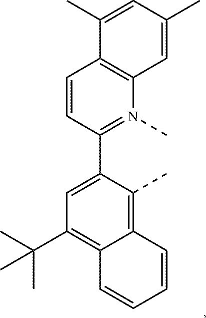

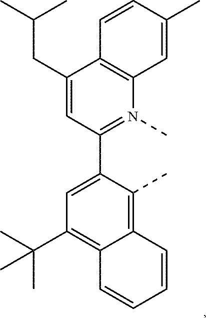

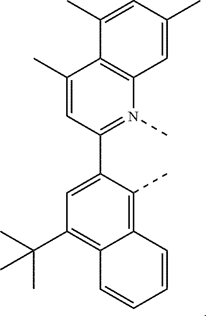

According to an embodiment of the present disclosure, in the electroluminescent device, the at least one phosphorescent material is an Ir complex and comprises the ligand L_{b}, wherein L_{b} is, at each occurrence identically or differently, selected from the group consisting of the following structures:

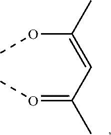

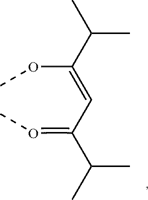

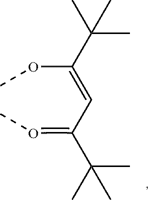

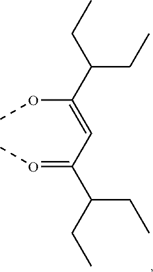

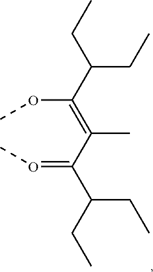

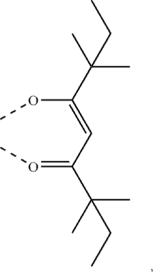

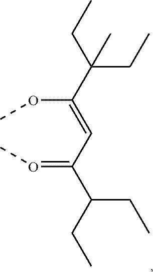

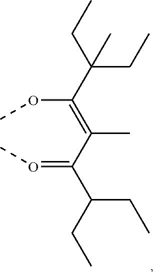

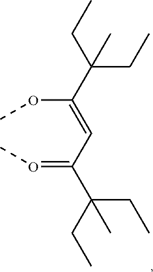

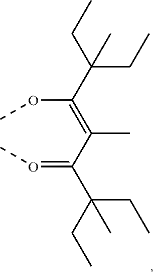

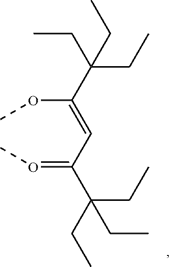

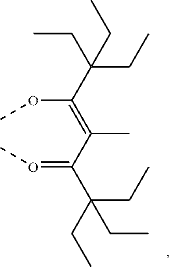

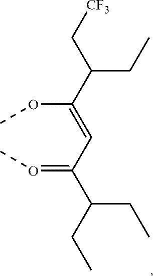

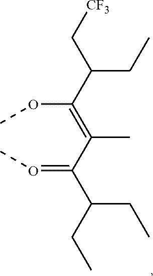

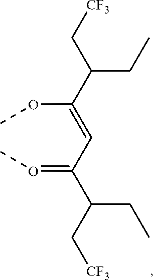

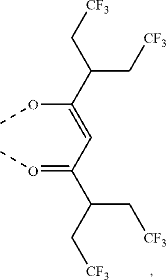

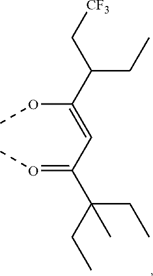

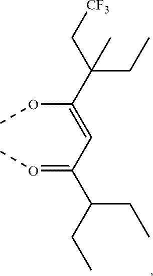

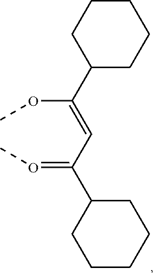

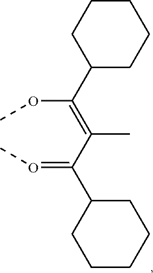

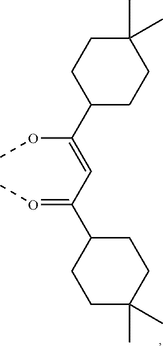

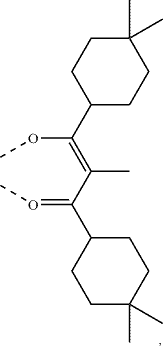

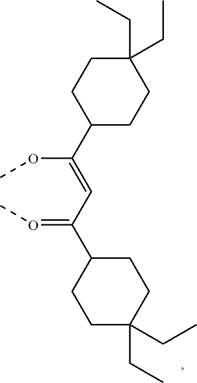

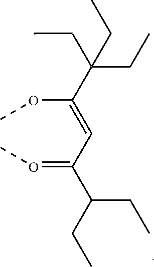

According to an embodiment of the present disclosure, in the electroluminescent device, the at least one phosphorescent material is selected from the group consisting of the following structures:

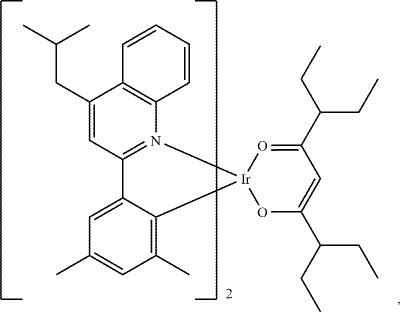

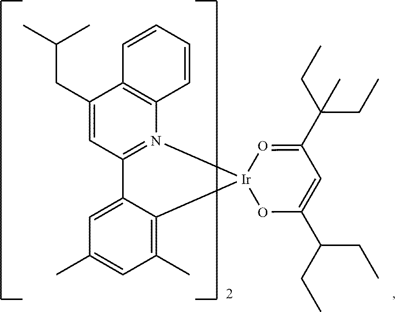

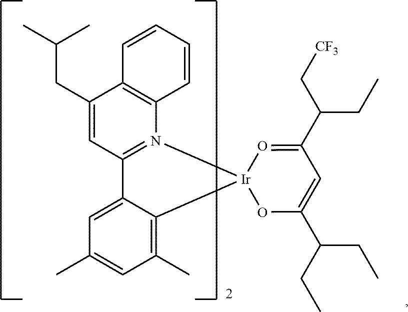

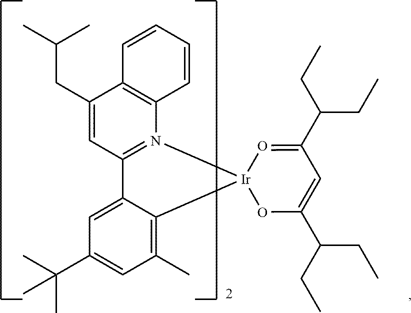

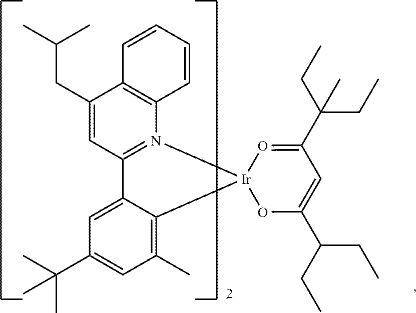

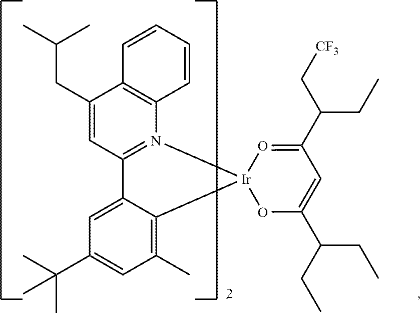

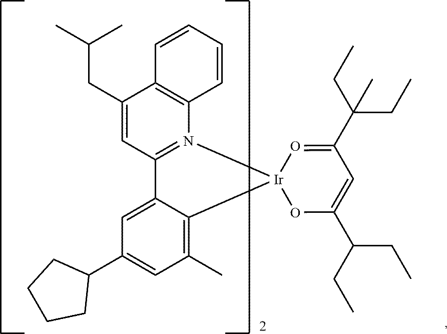

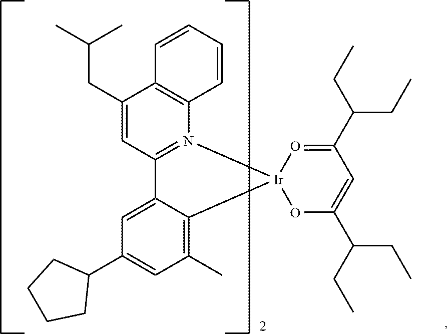

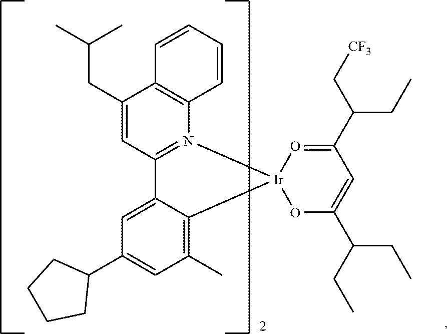

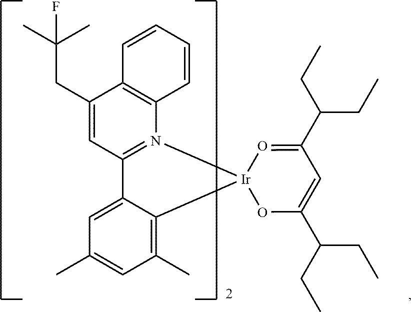

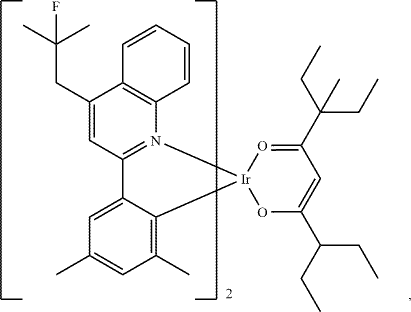

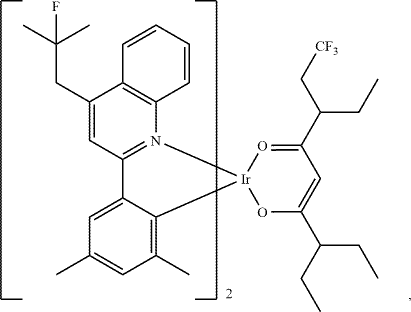

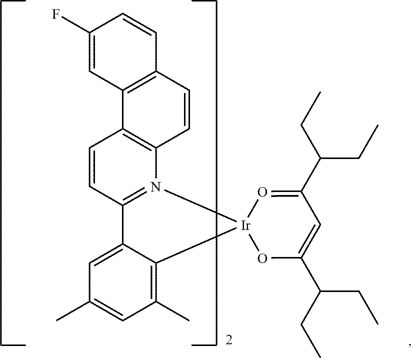

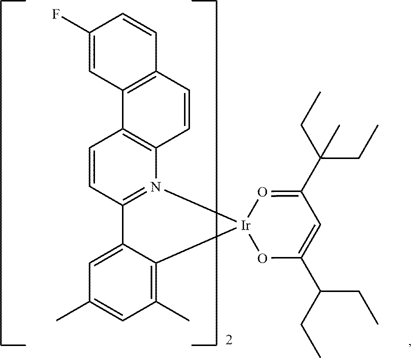

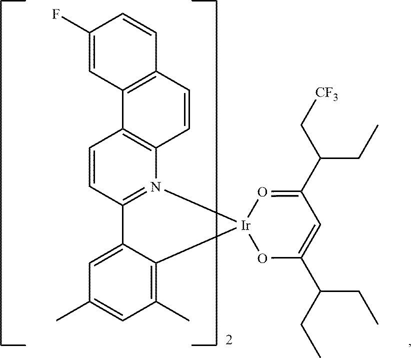

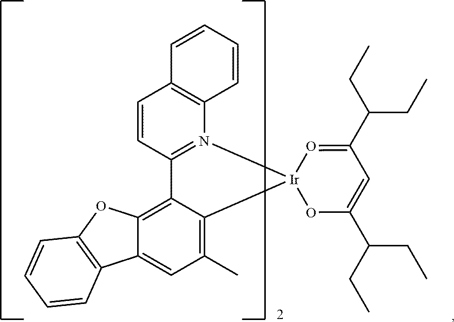

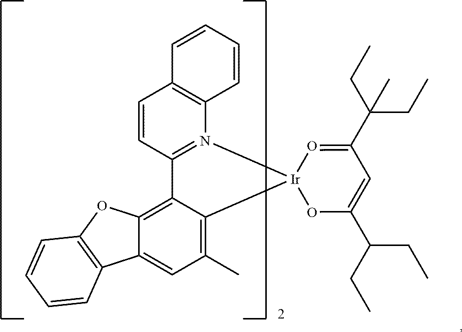

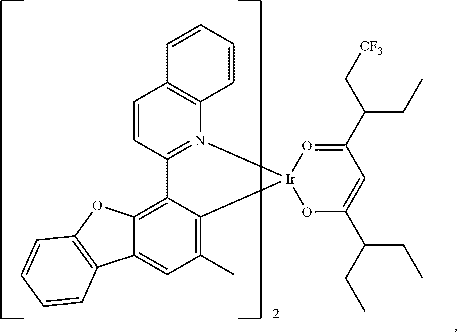

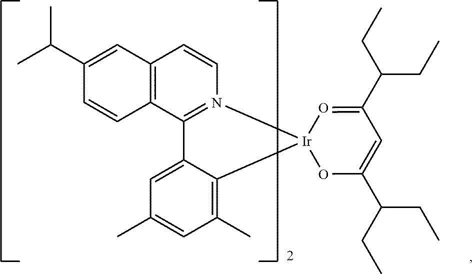

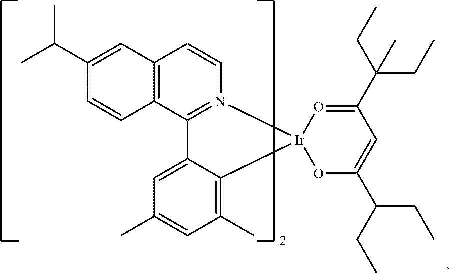

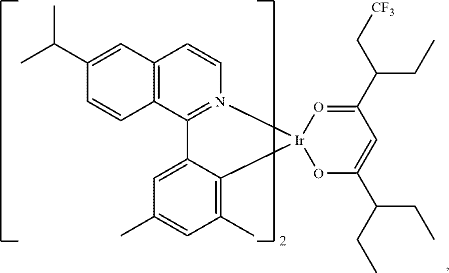

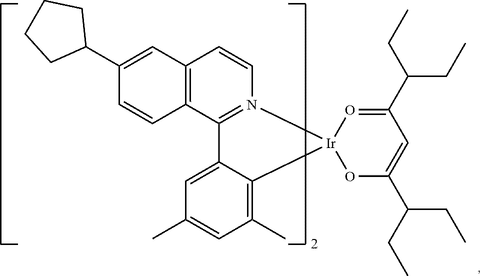

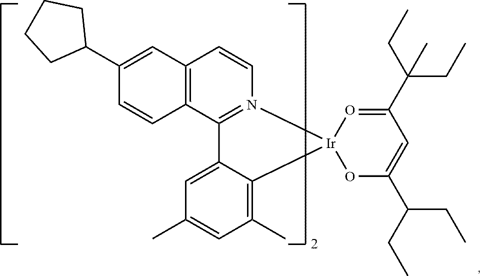

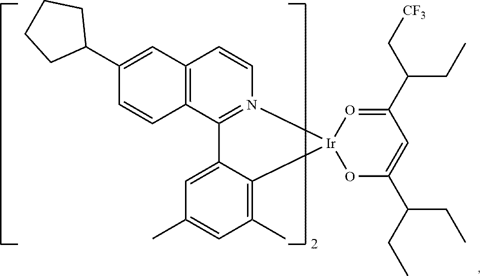

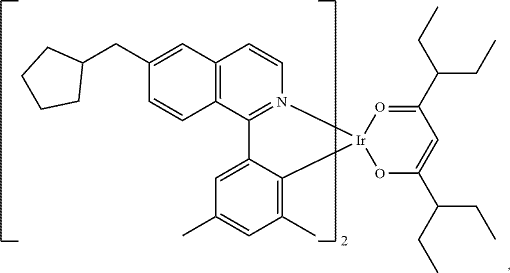

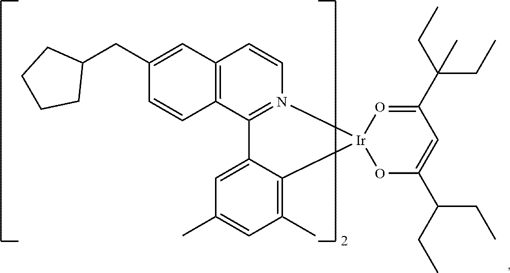

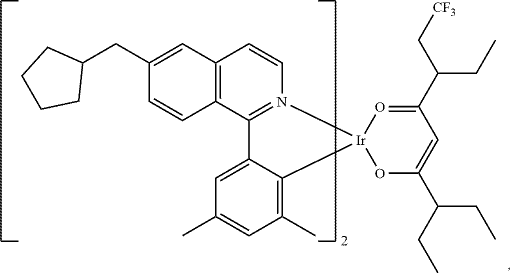

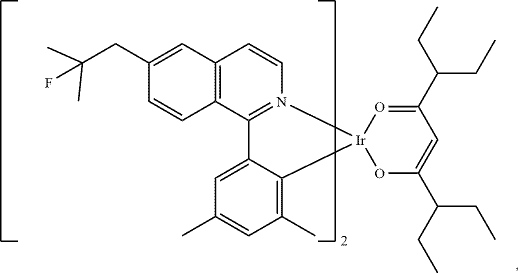

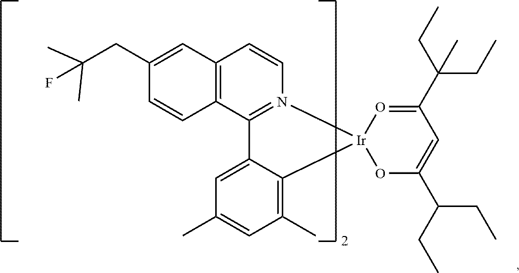

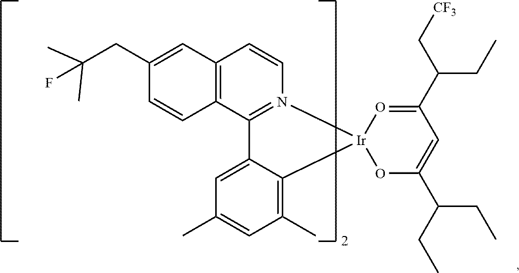

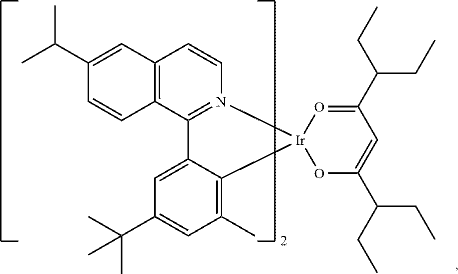

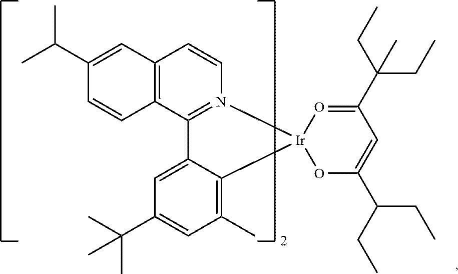

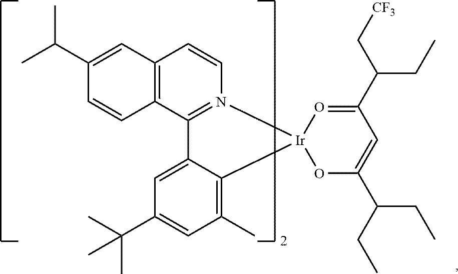

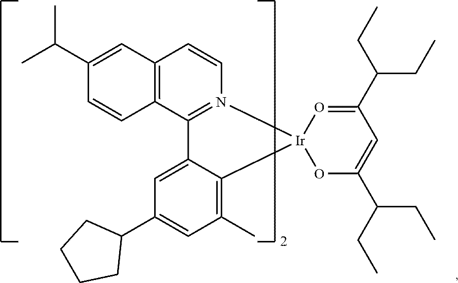

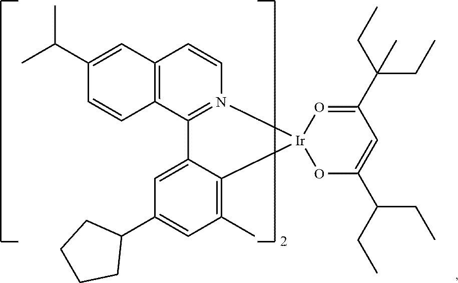

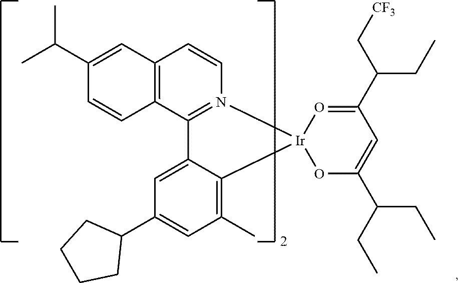

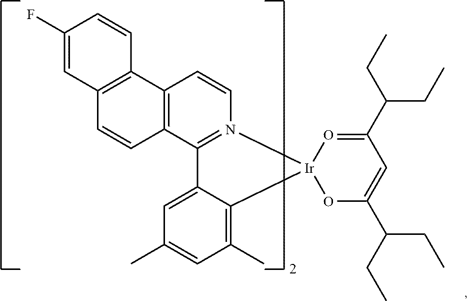

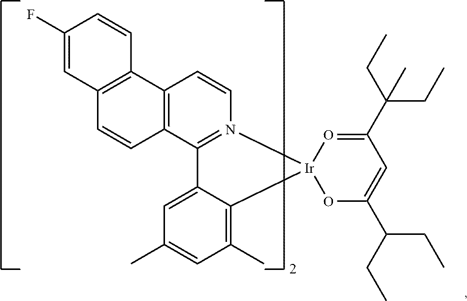

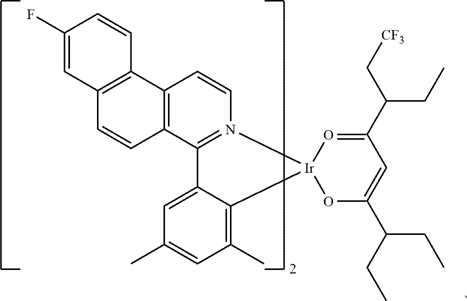

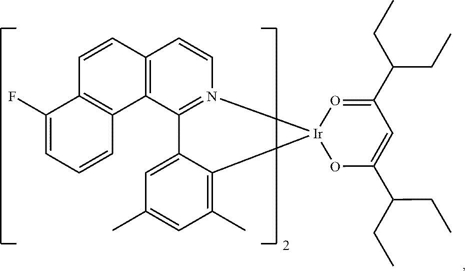

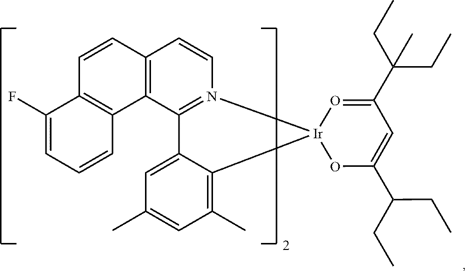

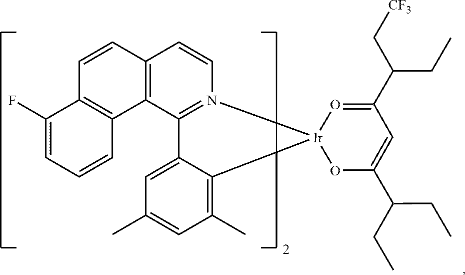

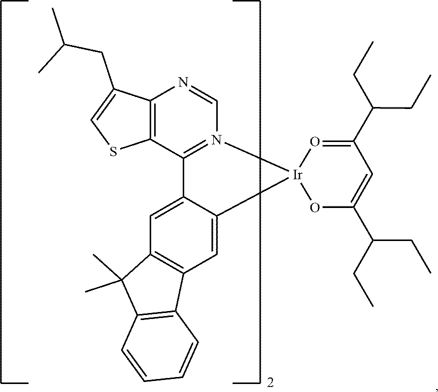

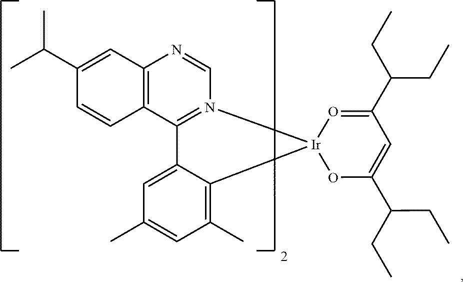

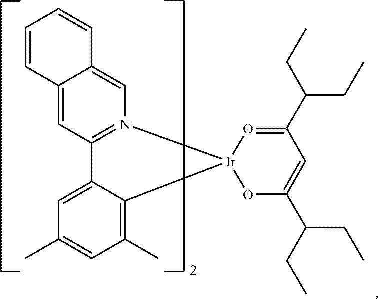

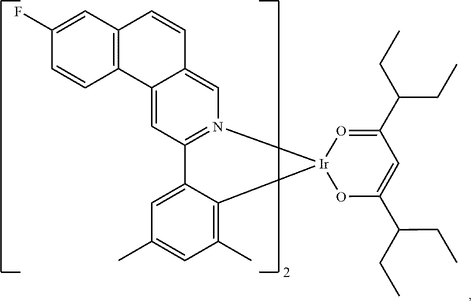

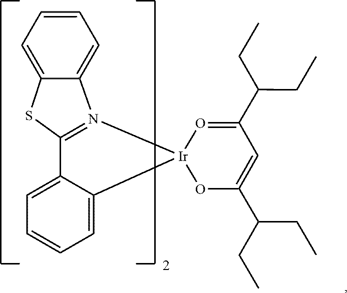

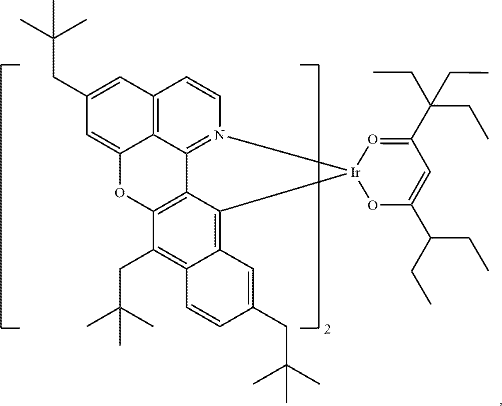

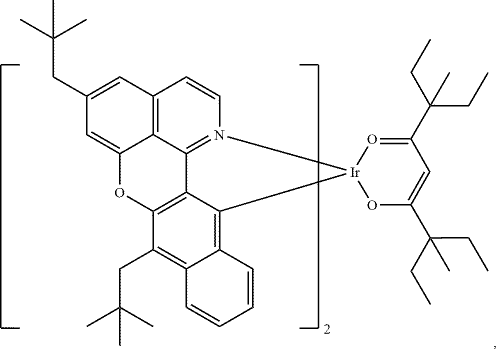

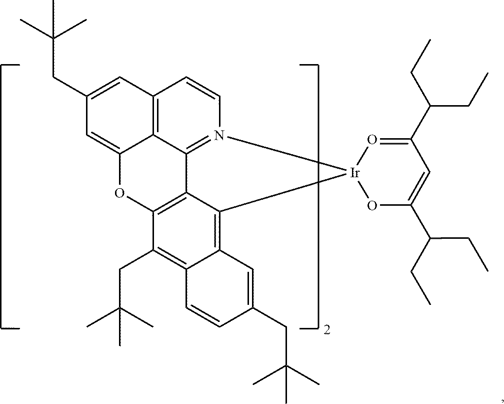

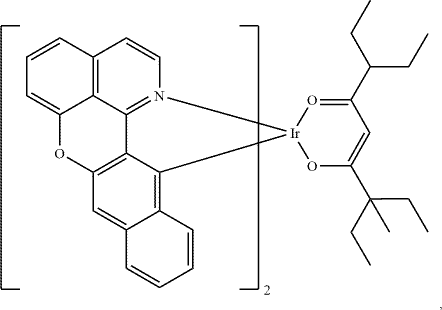

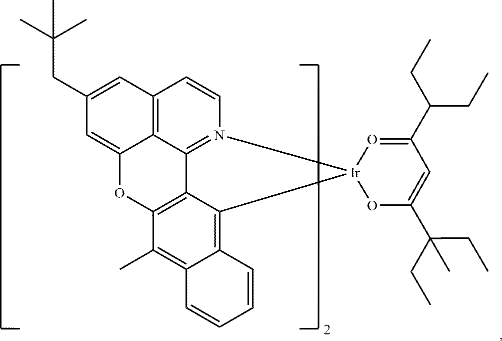

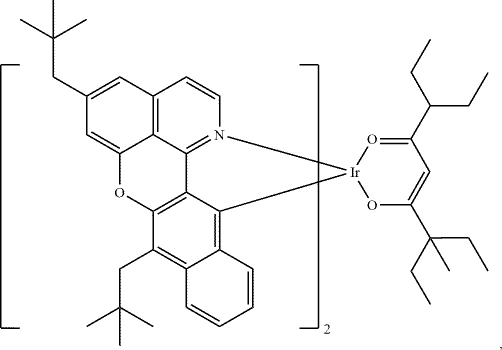

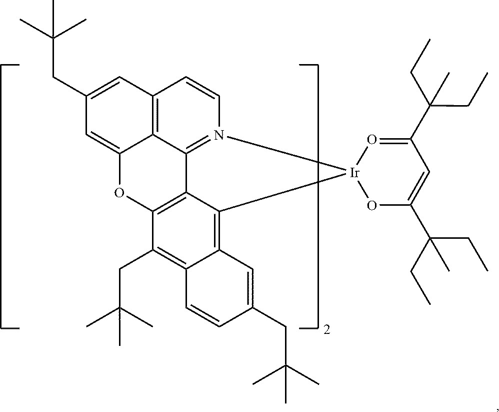

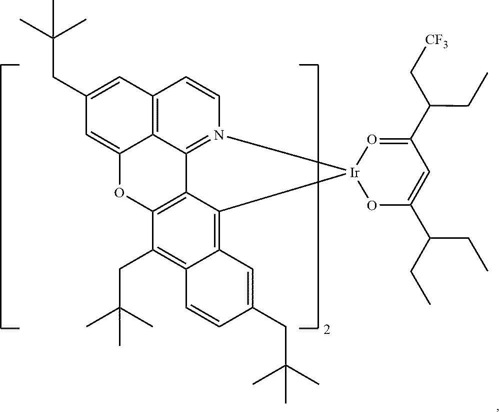

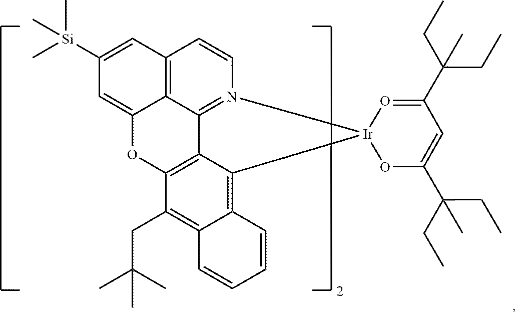

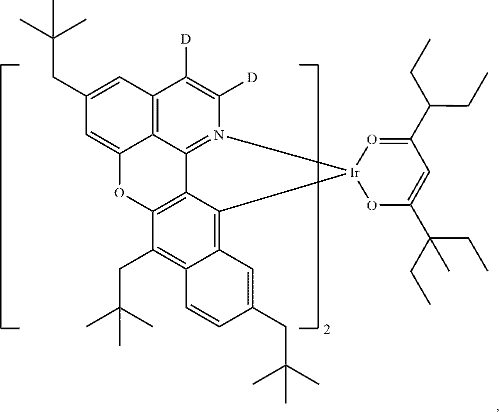

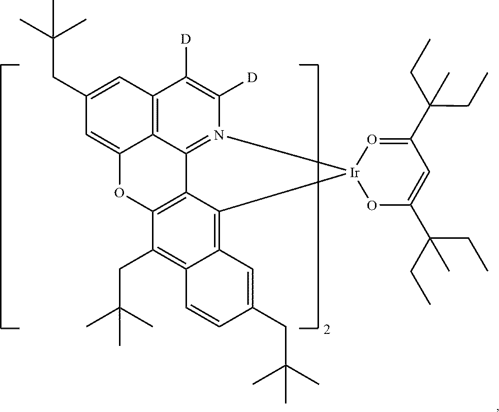

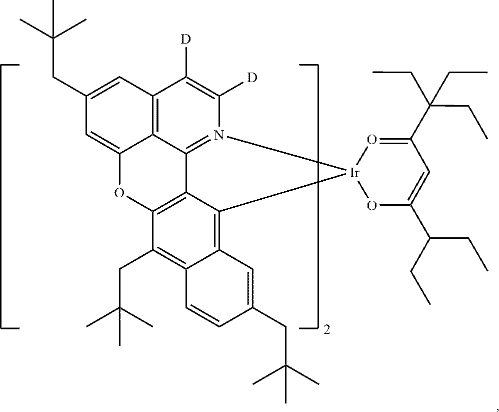

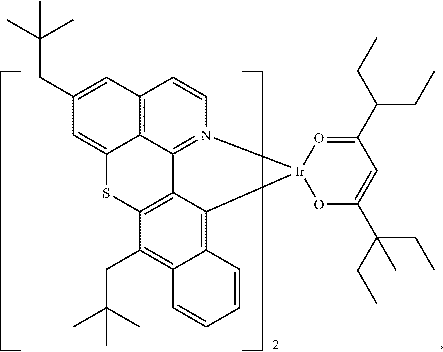

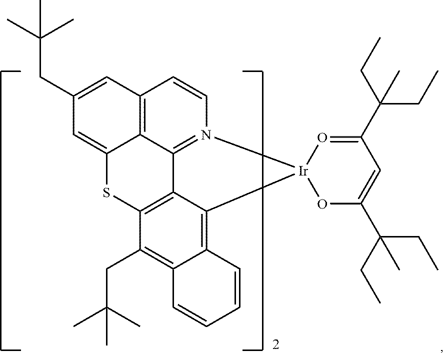

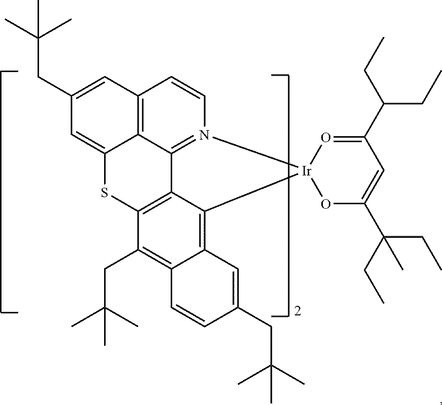

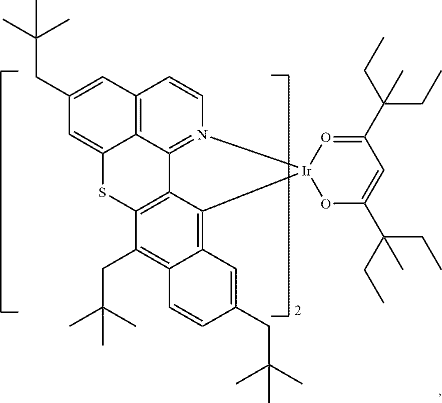

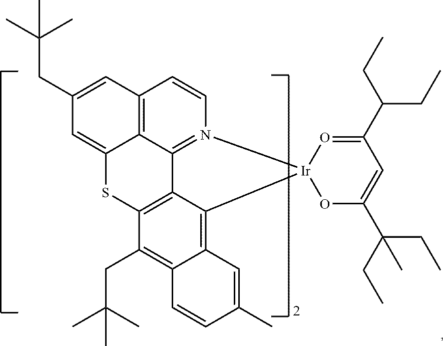

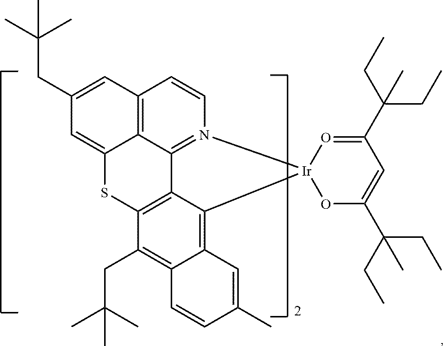

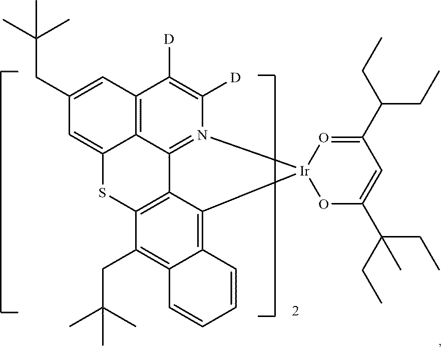

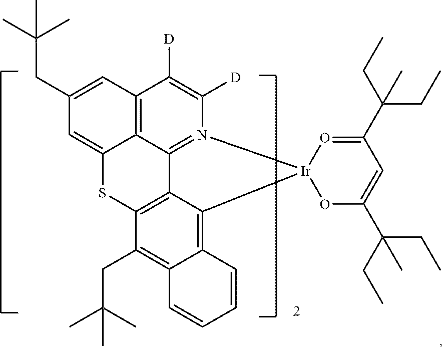

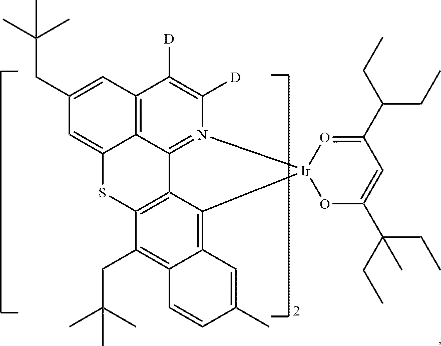

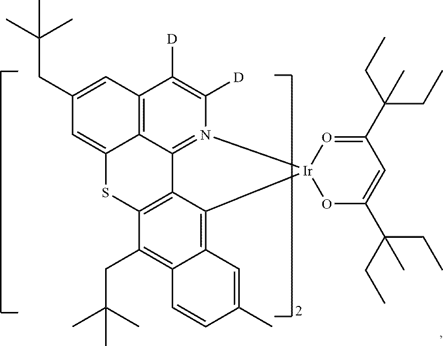

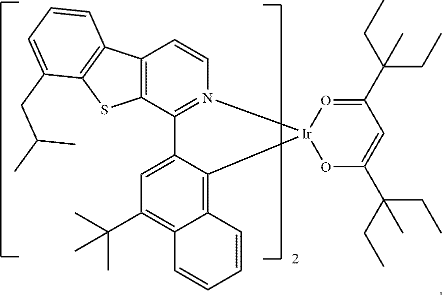

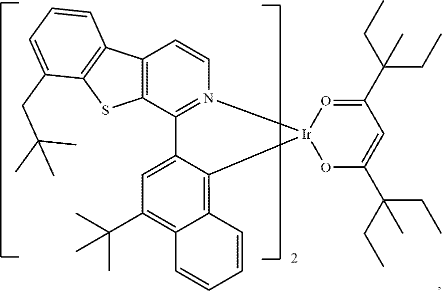

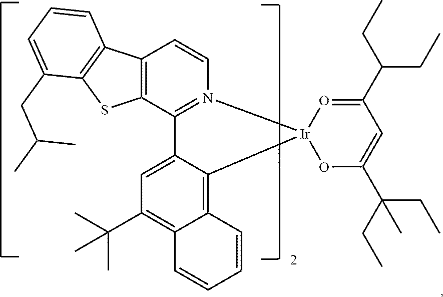

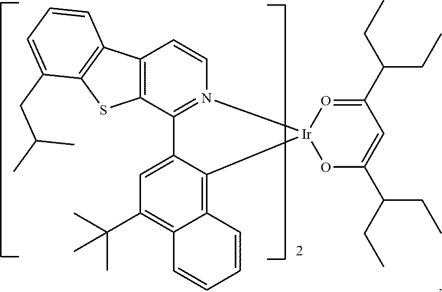

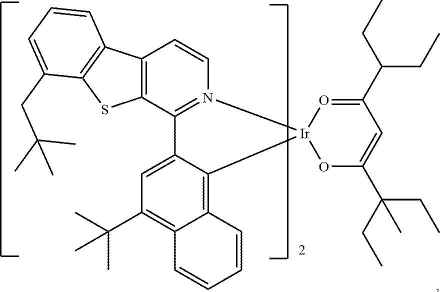

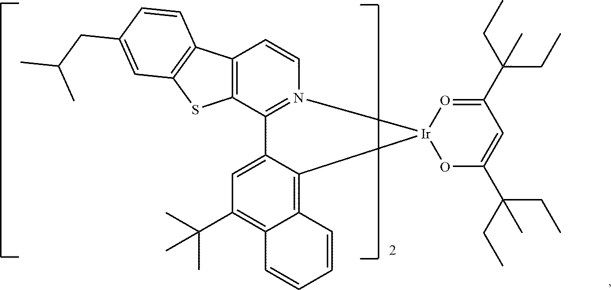

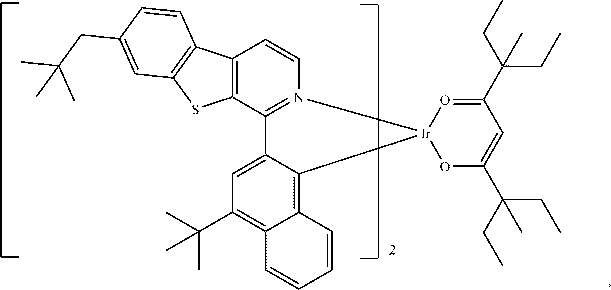

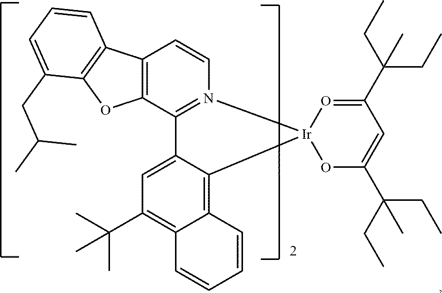

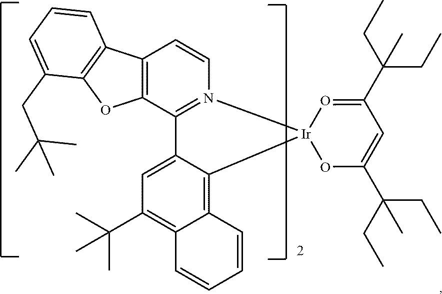

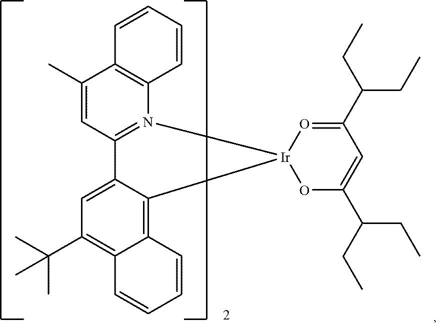

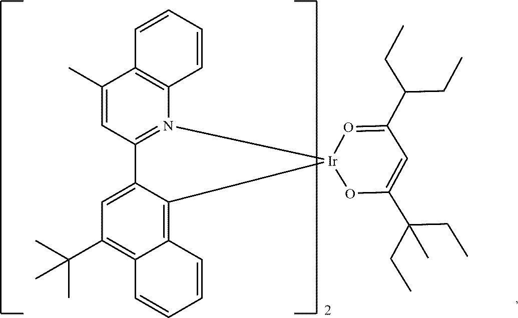

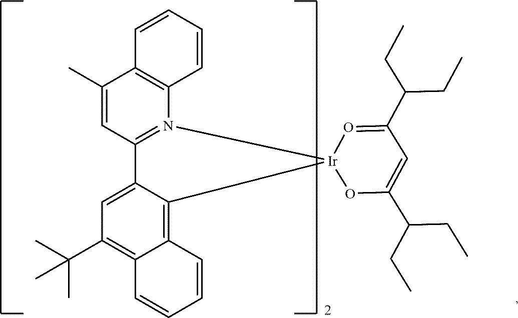

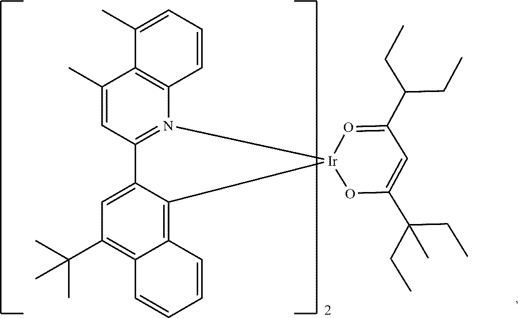

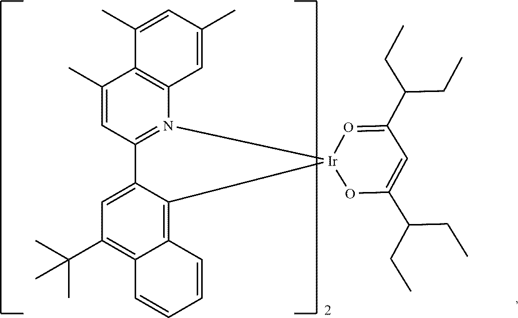

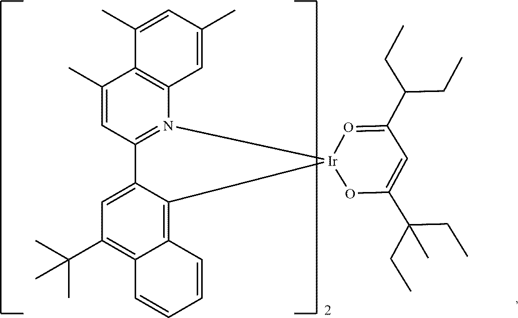

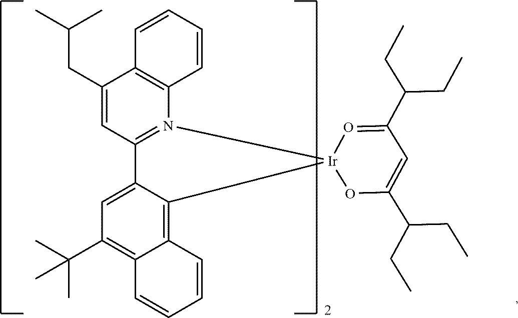

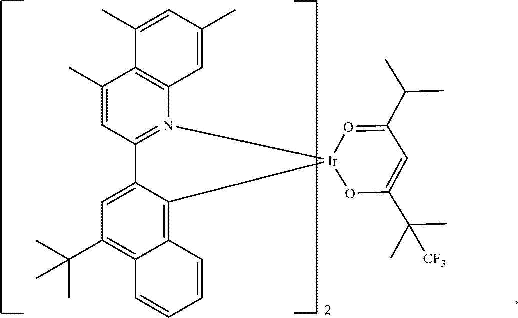

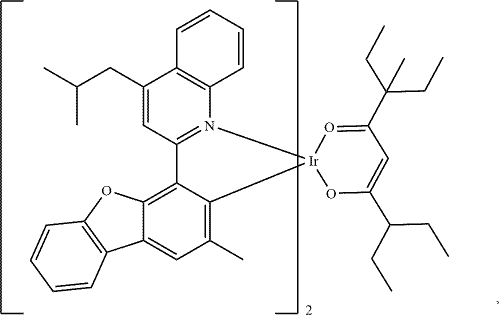

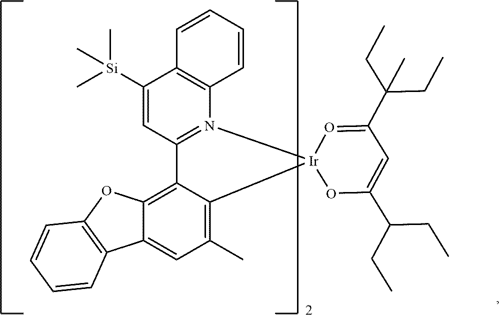

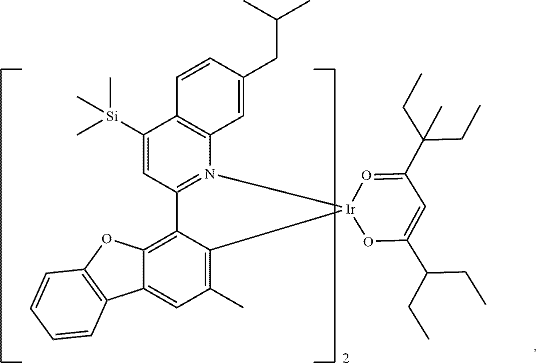

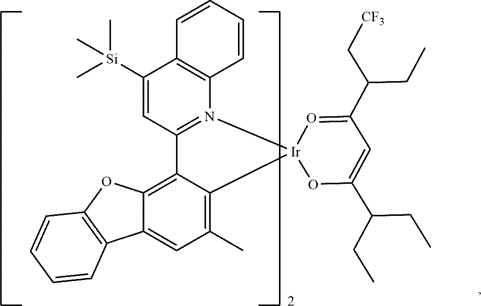

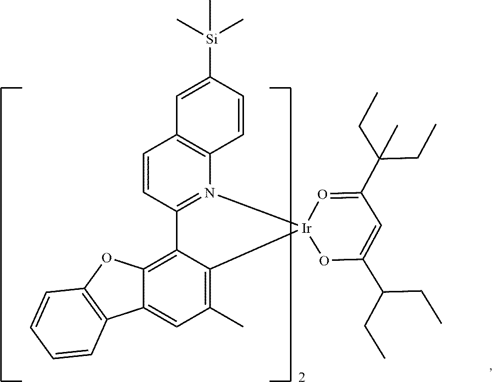

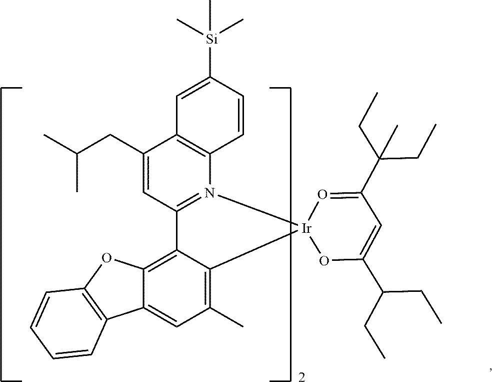

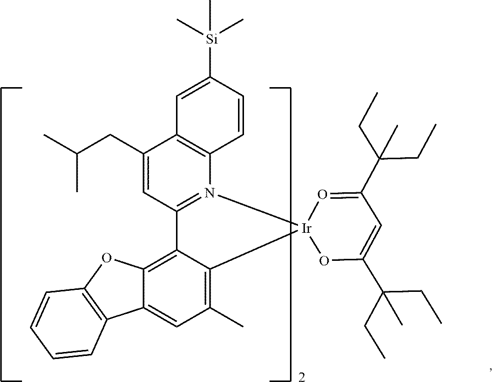

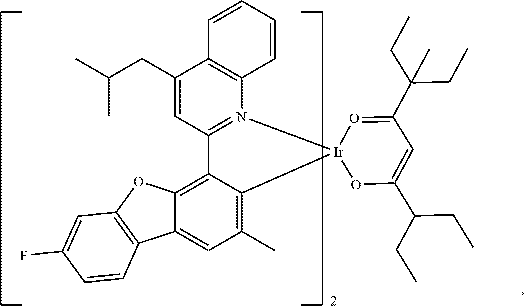

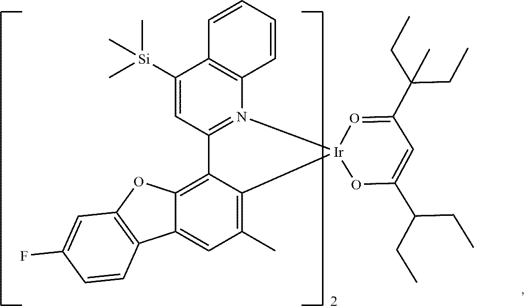

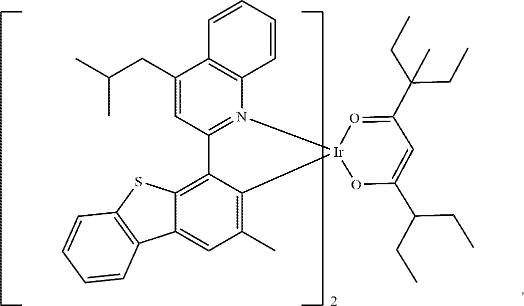

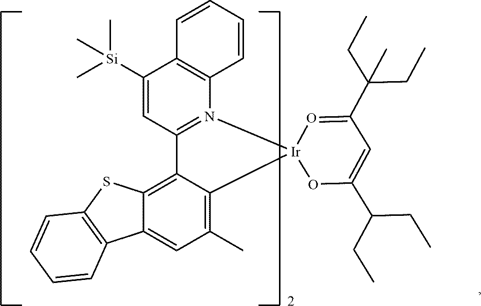

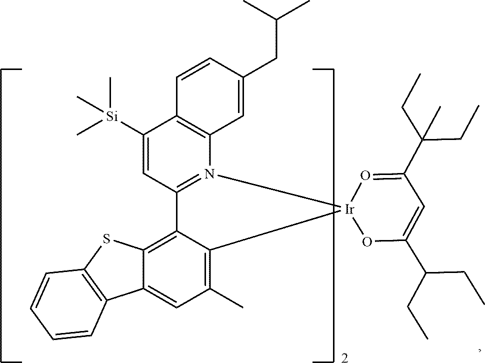

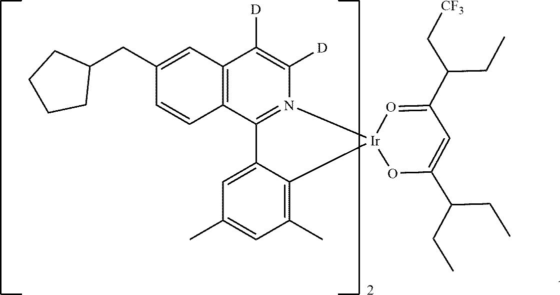

According to another embodiment of the present disclosure, further disclosed is an electronic apparatus, which comprises an electroluminescent device. A specific structure of the electroluminescent device is shown in any one of the preceding embodiments.

According to another embodiment of the present disclosure, further disclosed is a compound combination, which comprises a first compound and a second compound, wherein specific structures of the first compound and the second compound are shown in any one of the preceding embodiments.

### Combination With Other Materials

The materials described in the present disclosure for a particular layer in an organic light-emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

Methods for preparing a first compound and a second compound selected herein are not limited in the present disclosure. Those skilled in the art can prepare the first compound and the second compound by conventional synthesis methods or can easily prepare the first compound and the second compound with reference to Patent Application No. CN202110464197.4. The preparation methods are not repeated herein. The method for preparing an organic electroluminescent device is not limited. The preparation methods in the following device examples are merely examples and not to be construed as limitations. Those skilled in the art can make reasonable improvements on the preparation methods in the following device examples based on the related art. Exemplarily, the proportions of the first compound and the second compound are not particularly limited. Those skilled in the art can reasonably select the proportions within a certain range based on the related art. For example, taking the total weight of the materials in the light-emitting layer as reference, the total weight of the first compound and the second compound accounts for 99.5% to 80.0% of the total weight of the light-emitting layer, and a weight ratio of the first compound and the second compound is 1:99 to 99:1; or the weight ratio of the first compound and the second compound may be 20:80 to 99:1; or the weight ratio of the first compound and the second compound may be 50:50 to 90:10. In the preparation of a device, when two or more than two host materials together with a luminescent material are to be co-deposited to form a luminescent layer, this may be implemented through either of the following manners: (1) co-depositing the two or more than two host materials and the luminescent material from respective evaporation sources, to form the luminescent layer; or (2) pre-mixing the two or more than two host materials to obtain a pre-mixture, and co-depositing the pre-mixture from an evaporation source with the luminescent material from another evaporation source, to form the light-emitting layer. The latter pre-mixing method further save evaporation sources. In the present disclosure, it may be implemented through either of the following manners: (1) co-depositing the first host material, the second host material and the luminescent material from respective evaporation sources, to form the luminescent layer; or (2) pre-mixing the first host material and the second host material to obtain a pre-mixture, and co-depositing the pre-mixture from an evaporation source with the luminescent material from another evaporation source, to form the light-emitting layer. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well-known to the persons skilled in the art. As the persons skilled in the art are aware of the above-mentioned equipment use, test methods and other related contents, the inherent data of the sample can be obtained with certainty and without influence, so the above related contents are not further described in this patent.

### Device Example 1

First, a glass substrate having an indium tin oxide (ITO) anode with a thickness of 120 nm was cleaned and then treated with UV ozone and oxygen plasma. After the treatment, the substrate was dried in a nitrogen-filled glovebox to remove moisture. Then, the substrate was mounted on a substrate holder and placed in a vacuum chamber. Organic layers specified below were sequentially deposited through vacuum thermal evaporation on the ITO anode at a rate of 0.01 to 5 Å/s and a vacuum degree of about 10⁻⁸ torr. Compound HI was used as a hole injection layer (HIL) with a thickness of 100 Å. Compound HT was used as a hole transporting layer (HTL) with a thickness of 400 Å. Compound EB was used as an electron blocking layer (EBL) with a thickness of 50 Å. Then, Compound 1-333 as a first host, Compound 2-229 as a second host and Compound RD as a dopant were co-deposited as an emissive layer (EML) with a thickness of 400 Å. Compound HB was used as a hole blocking layer (HBL) with a thickness of 50 Å. On the hole blocking layer, Compound ET and 8-hydroxyquinolinolato-lithium (Liq) were co-deposited as an electron transporting layer (ETL) with a thickness of 350 Å. Finally, 8-hydroxyquinolinolato-lithium (Liq) was deposited as an electron injection layer (EIL) with a thickness of 10 Å, and A1 was deposited as a cathode with a thickness of 1200 Å. The device was transferred back to the glovebox and encapsulated with a glass lid to complete the device.

### Device Example 2

The implementation mode in Device Example 2 was the same as that in Device Example 1, except that in the emissive layer (EML), Compound 2-229 was replaced with Compound 2-283 as the second host.

### Device Example 3

The implementation mode in Device Example 3 was the same as that in Device Example 1, except that in the emissive layer (EML), Compound 2-229 was replaced with Compound 2-205 as the second host.

### Device Example 4

The implementation mode in Device Example 4 was the same as that in Device Example 1, except that in the emissive layer (EML), Compound 2-229 was replaced with Compound 2-227 as the second host.

### Device Example 5

The implementation mode in Device Example 5 was the same as that in Device Example 1, except that in the emissive layer (EML), Compound 2-229 was replaced with Compound 2-275 as the second host.

### Device Comparative Example 1

The implementation mode in Device Comparative Example 1 was the same as that in Device Example 1, except that in the emissive layer (EML), Compound 1-333 and Compound 2-229 were replaced with Compound 1-333 as a single host and a weight ratio of Compound 1-333 and Compound RD was 97:3.

Detailed structures and thicknesses of layers of the devices are shown in the following table. A layer using more than one material is obtained by doping different compounds at their weight ratio as recorded.

TABLE 1

Part of device structures in device examples and device comparative example

| | | | | | | |
|---|---|---|---|---|---|---|
| Device ID | HIL | HTL | EBL | EML | HBL | ETL |
| Example 1 | Compound HI (100 Å) | Compound HT (400 Å) | Compound EB (50 Å) | Compound 1-333:Compound 2-229:Compound RD (87.3:9.7:3) (400 Å) | Compound HB (50 Å) | Compound ET:Liq (40:60) (350 Å) |
| Example 2 | Compound HI (100 Å) | Compound HT (400 Å) | Compound EB (50 Å) | Compound 1-333:Compound 2-283:Compound RD (87.3:9.7:3) (400 Å) | Compound HB (50 Å) | Compound ET:Liq (40:60) (350 Å) |
| Example 3 | Compound HI (100 Å) | Compound HT (400 Å) | Compound EB (50 Å) | Compound 1-333:Compound 2-205:Compound RD (87.3:9.7:3) (400 Å) | Compound HB (50 Å) | Compound ET:Liq (40:60) (350 Å) |
| Example 4 | Compound HI (100 Å) | Compound HT (400 Å) | Compound EB (50 Å) | Compound 1-333:Compound 2-227:Compound RD (87.3:9.7:3) (400 Å) | Compound HB (50 Å) | Compound ET:Liq (40:60) (350 Å) |
| Example 5 | Compound HI (100 Å) | Compound HT (400 Å) | Compound EB (50 Å) | Compound 1-333:Compound 2-275:Compound RD (87.3:9.7:3) | Compound HB (50 Å) | Compound ET:Liq (40:60) (350 Å) |
| Compar ative Example 1 | Compound HI (100 Å) | Compound HT (400 Å) | Compound EB (50 Å) | Compound 1-333:Compound RD (97:3) (400 Å) | Compound HB (50 Å) | Compound ET:Liq (40:60) (350 Å) |

The structures of the materials used in the devices are shown as follows:

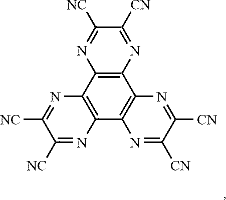

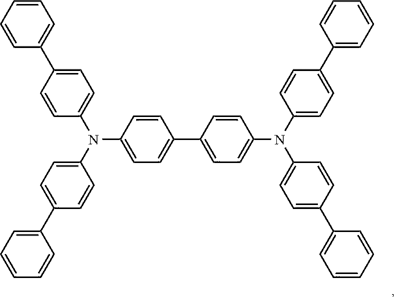

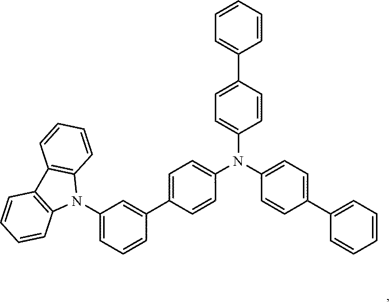

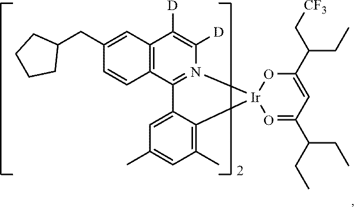

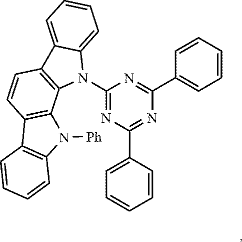

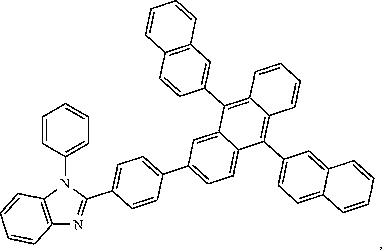

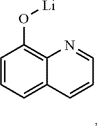

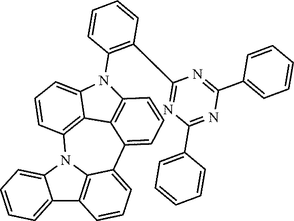 [SMILES: c1ccc(-c2nc(-c3ccccc3)nc(-c3ccccc3-n3c4cccc5c6cccc7c8ccccc8n(c8cccc3c8c54)c67)n2)cc1]

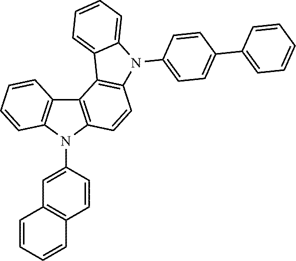 [SMILES: c1ccc(-c2ccc(-n3c4ccccc4c4c5c6ccccc6n(-c6ccc7ccccc7c6)c5ccc43)cc2)cc1]

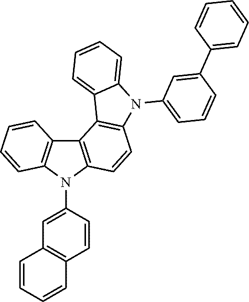 [SMILES: c1ccc(-c2cccc(-n3c4ccccc4c4c5c6ccccc6n(-c6ccc7ccccc7c6)c5ccc43)c2)cc1]

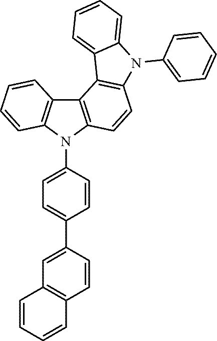 [SMILES: c1ccc(-n2c3ccccc3c3c4c5ccccc5n(-c5ccc(-c6ccc7ccccc7c6)cc5)c4ccc32)cc1]

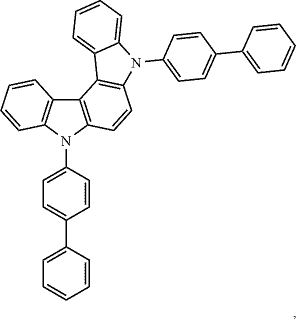

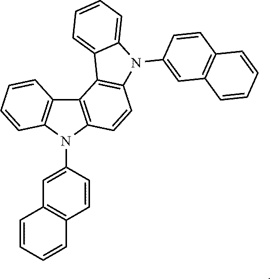

The maximum emission wavelength (λₘₐₓ), CIE data and current efficiency (CE) of the device examples and device comparative example were measured at a constant current of 15 mA/cm². The device lifetime (LT97) was measured at a constant current of 80 mA/cm², where LT97 is the time for the device to decay to 97% of its initial brightness. The data was recorded and shown in Table 2.

TABLE 2

Device data

| Device ID | CIE (x,y) | λₘₐₓ (nm) | CE [cd/A] | LT97 [h] |
|---|---|---|---|---|
| Example 1 | 0.678, 0.321 | 622 | 24.3 | 187 |
| Example 2 | 0.678, 0.321 | 621 | 24.3 | 163 |
| Example 3 | 0.678, 0.321 | 620 | 24.3 | 186 |
| Example 4 | 0.678, 0.321 | 621 | 23.8 | 175 |
| Example 5 | 0.678, 0.321 | 621 | 24.1 | 241 |
| Comparative Example 1 | 0.679, 0.321 | 622 | 23.8 | 75 |

### Discussion

As can be seen from the data in Table 2, the CIE data and maximum emission wavelength of the examples and comparative example remain substantially consistent; in terms of current efficiency, Examples 1 to 5 can maintain substantially the same high efficiency as Comparative Example 1 or have a further improvement in the high efficiency level of Comparative Example 1; more importantly, in terms of lifetime, compared to that in Comparative Example 1, the lifetimes in Examples 1 to 5 are improved by 112 hours, 88 hours, 111 hours, 100 hours and 166 hours, respectively, and improved by as many as 2.5 times, 2.2 times, 2.5 times, 2.3 times and 3.2 times, respectively. As can be seen from the above data, compared to the case where the first compound is used alone, the electroluminescent device comprising the combination of the first compound and the second compound selected in the present disclosure has a significant improvement in device performance, and the device lifetime is significantly improved while maintaining high efficiency or further improving the device efficiency, which fully indicates that the first compound and the second compound selected in the present disclosure have excellent device characteristics and great application potential when used in combination.

It should be understood that various embodiments described herein are merely examples and not intended to limit the scope of the present disclosure. Therefore, it is apparent to the persons skilled in the art that the present disclosure as claimed may include variations from specific embodiments and preferred embodiments described herein. Many of materials and structures described herein may be substituted with other materials and structures without departing from the spirit of the present disclosure. It should be understood that various theories as to why the present disclosure works are not intended to be limitative.

What is claimed is:

1. An electroluminescent device, comprising:
an anode,
a cathode, and
an organic layer disposed between the anode and the cathode, wherein the organic layer at least comprises a first compound and a second compound;
wherein the first compound has a structure of H-L-E, wherein H has a structure represented by Formula 1: 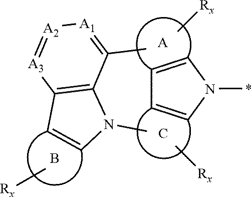
wherein in Formula 1, A₁, A₂ and A₃ are, at each occurrence identically or differently, selected from N or CR, and the ring A, the ring B and the ring C are, at each occurrence identically or differently, selected from a carbocyclic ring having 5 to 18 carbon atoms or a heterocyclic ring having 3 to 18 carbon atoms; and
Rₓ represents, at each occurrence identically or differently, mono-substitution, multiple substitutions or non-substitution;
wherein E has a structure represented by Formula 1-a: 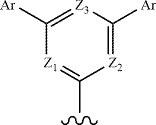
wherein in Formula 1-a, Ar is, at each occurrence identically or differently, selected from substituted or unsubstituted aryl having 6 to 30 carbon atoms or substituted or unsubstituted heteroaryl having 3 to 30 carbon atoms;
Z₁ to Z₃ are each independently selected from N or CR_{z}, and at least one of Z₁ to Z₃ is N;
L is selected from a single bond, substituted or unsubstituted arylene having 6 to 30 carbon atoms, substituted or unsubstituted heteroarylene having 3 to 30 carbon atoms or a combination thereof;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R, Rₓ can be optionally joined to form a ring;
wherein the second compound has a structure represented by Formula 2: 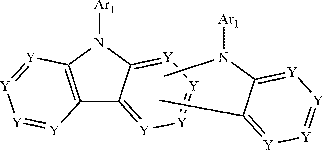
wherein Y is, at each occurrence identically or differently, selected from C, CR_{Y} or N;
R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.

2. The electroluminescent device according to claim 1, wherein H has a structure represented by Formula 1A: 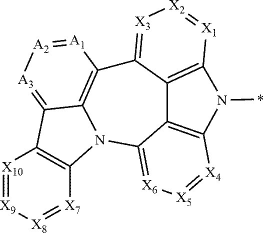
wherein A₁ to A₃ are, at each occurrence identically or differently, selected from N or CR, and X₁ to X₁₀ are, at each occurrence identically or differently, selected from N or CRₓ;
R and Rₓ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R, Rₓ can be optionally joined to form a ring; and
preferably, R and Rₓ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted amino having 0 to 20 carbon atoms, a cyano group, an isocyano group, a hydroxyl group, a sulfanyl group and combinations thereof.

3. The electroluminescent device according to claim 1, wherein at least one of R and Rₓ is selected from deuterium, halogen, a cyano group, a hydroxyl group, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms or a combination thereof;
adjacent substituents R and Rₓ can be optionally joined to form a ring; and
preferably, at least one of R and Rₓ is selected from deuterium, fluorine, a cyano group, a hydroxyl group, a sulfanyl group, methyl, trideuteromethyl, vinyl, phenyl, biphenyl, naphthyl, 4-cyanophenyl, dibenzofuranyl, dibenzothienyl, triphenylenyl, carbazolyl, 9-phenylcarbazolyl, 9,9-dimethylfluorenyl, pyridyl, phenylpyridyl or a combination thereof.

4. The electroluminescent device according to claim 1, wherein H is selected from the group consisting of the following structures: 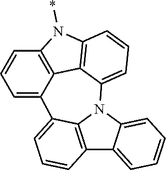 [SMILES: *n1c2cccc3c4cccc5c6ccccc6n(c6cccc1c6c32)c45] 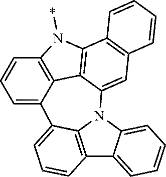 [SMILES: *n1c2cccc3c4cccc5c6ccccc6n(c6cc7ccccc7c1c6c32)c45] 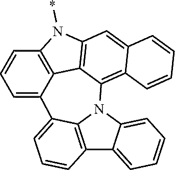 [SMILES: *n1c2cccc3c4cccc5c6ccccc6n(c45)c4c5ccccc5cc1c4c32] 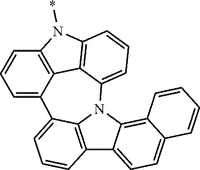 [SMILES: *n1c2cccc3c4cccc5c6ccc7ccccc7c6n(c6cccc1c6c32)c45] 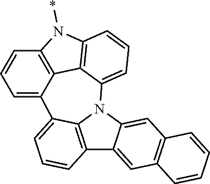 [SMILES: *n1c2cccc3c4cccc5c6cc7ccccc7cc6n(c6cccc1c6c32)c45] 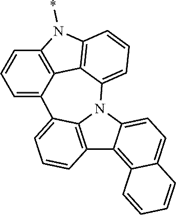 [SMILES: *n1c2cccc3c4cccc5c6c7ccccc7ccc6n(c6cccc1c6c32)c45] 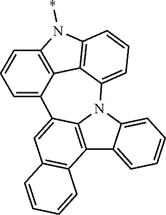 [SMILES: *n1c2cccc3c4cc5ccccc5c5c6ccccc6n(c6cccc1c6c32)c45] 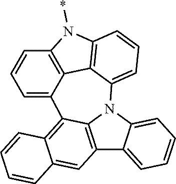 [SMILES: *n1c2cccc3c2c2c1cccc2n1c2ccccc2c2cc4ccccc4c3c21] 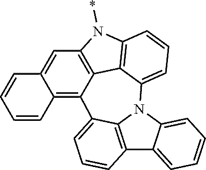 [SMILES: *n1c2cc3ccccc3c3c4cccc5c6ccccc6n(c6cccc1c6c32)c54] 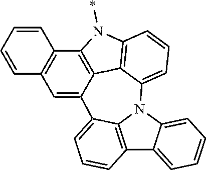 [SMILES: *n1c2cccc3c2c2c(cc4ccccc4c21)c1cccc2c4ccccc4n3c12] 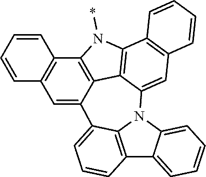 [SMILES: *n1c2c3ccccc3cc3c4cccc5c6ccccc6n(c6cc7ccccc7c1c6c32)c45] 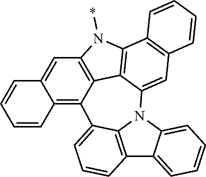 [SMILES: *n1c2cc3ccccc3c3c4cccc5c6ccccc6n(c6cc7ccccc7c1c6c32)c54] 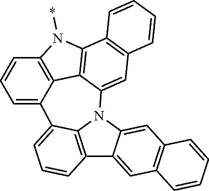 [SMILES: *n1c2cccc3c4cccc5c6cc7ccccc7cc6n(c6cc7ccccc7c1c6c32)c45] 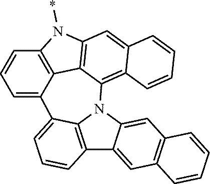 [SMILES: *n1c2cccc3c4cccc5c6cc7ccccc7cc6n(c45)c4c5ccccc5cc1c4c32] 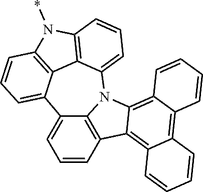 [SMILES: *n1c2cccc3c4cccc5c6c7ccccc7c7ccccc7c6n(c6cccc1c6c32)c45] 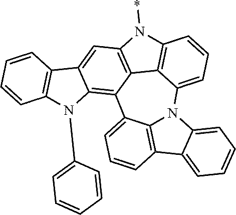 [SMILES: *n1c2cc3c4ccccc4n(-c4ccccc4)c3c3c4cccc5c6ccccc6n(c6cccc1c6c32)c54] 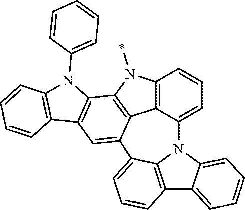 [SMILES: *n1c2cccc3c2c2c(cc4c5ccccc5n(-c5ccccc5)c4c21)c1cccc2c4ccccc4n3c12] 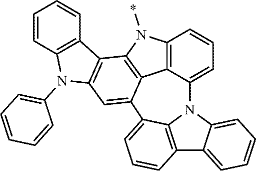 [SMILES: *n1c2cccc3c2c2c(cc4c(c5ccccc5n4-c4ccccc4)c21)c1cccc2c4ccccc4n3c12] 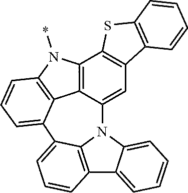 [SMILES: *n1c2cccc3c4cccc5c6ccccc6n(c6cc7c8ccccc8sc7c1c6c32)c45] 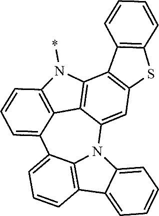 [SMILES: *n1c2cccc3c4cccc5c6ccccc6n(c6cc7sc8ccccc8c7c1c6c32)c45] 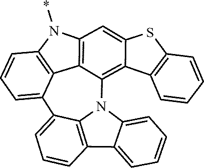 [SMILES: *n1c2cccc3c4cccc5c6ccccc6n(c45)c4c5c(cc1c4c32)sc1ccccc15] 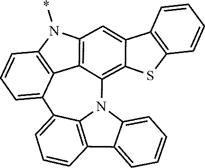 [SMILES: *n1c2cccc3c4cccc5c6ccccc6n(c45)c4c5sc6ccccc6c5cc1c4c32] 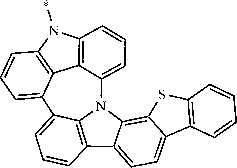 [SMILES: *n1c2cccc3c4cccc5c6ccc7c8ccccc8sc7c6n(c6cccc1c6c32)c45] 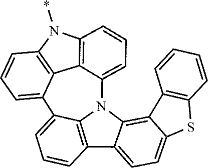 [SMILES: *n1c2cccc3c4cccc5c6ccc7sc8ccccc8c7c6n(c6cccc1c6c32)c45] 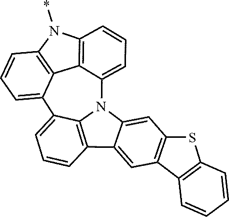 [SMILES: *n1c2cccc3c4cccc5c6cc7c(cc6n(c6cccc1c6c32)c45)sc1ccccc17] 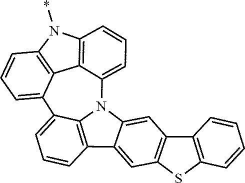 [SMILES: *n1c2cccc3c4cccc5c6cc7sc8ccccc8c7cc6n(c6cccc1c6c32)c45] 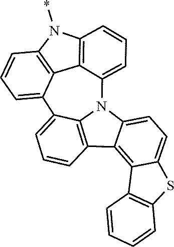 [SMILES: *n1c2cccc3c4cccc5c6c7c(ccc6n(c6cccc1c6c32)c45)sc1ccccc17] 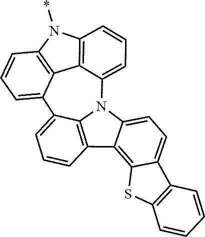 [SMILES: *n1c2cccc3c4cccc5c6c7sc8ccccc8c7ccc6n(c6cccc1c6c32)c45] 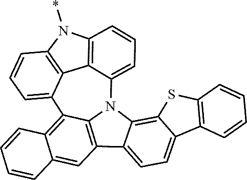 [SMILES: *n1c2cccc3c2c2c1cccc2n1c2c(ccc4c5ccccc5sc42)c2cc4ccccc4c3c21] 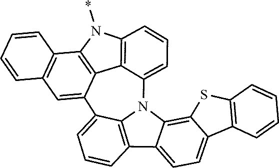 [SMILES: *n1c2cccc3c2c2c(cc4ccccc4c21)c1cccc2c4ccc5c6ccccc6sc5c4n3c12] 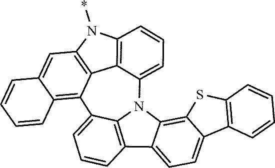 [SMILES: *n1c2cc3ccccc3c3c4cccc5c6ccc7c8ccccc8sc7c6n(c6cccc1c6c32)c54] 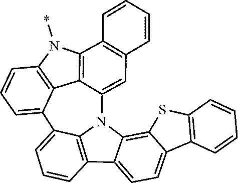 [SMILES: *n1c2cccc3c4cccc5c6ccc7c8ccccc8sc7c6n(c6cc7ccccc7c1c6c32)c45] 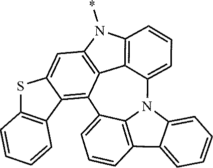 [SMILES: *n1c2cc3sc4ccccc4c3c3c4cccc5c6ccccc6n(c6cccc1c6c32)c54] 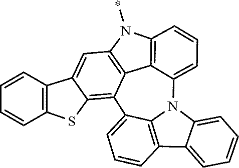 [SMILES: *n1c2cc3c4ccccc4sc3c3c4cccc5c6ccccc6n(c6cccc1c6c32)c54] 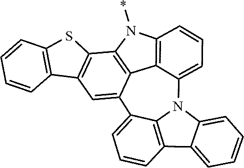 [SMILES: *n1c2cccc3c2c2c(cc4c5ccccc5sc4c21)c1cccc2c4ccccc4n3c12] 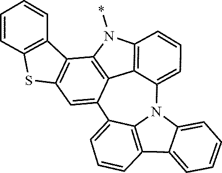 [SMILES: *n1c2cccc3c2c2c(cc4sc5ccccc5c4c21)c1cccc2c4ccccc4n3c12] 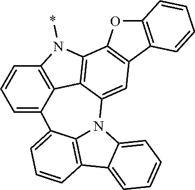 [SMILES: *n1c2cccc3c4cccc5c6ccccc6n(c6cc7c8ccccc8oc7c1c6c32)c45] 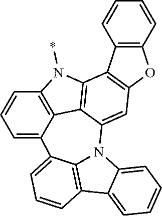 [SMILES: *n1c2cccc3c4cccc5c6ccccc6n(c6cc7oc8ccccc8c7c1c6c32)c45] 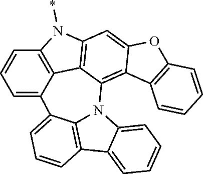 [SMILES: *n1c2cccc3c4cccc5c6ccccc6n(c45)c4c5c(cc1c4c32)oc1ccccc15] 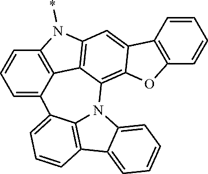 [SMILES: *n1c2cccc3c4cccc5c6ccccc6n(c45)c4c5oc6ccccc6c5cc1c4c32] 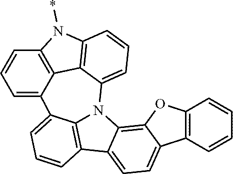 [SMILES: *n1c2cccc3c4cccc5c6ccc7c8ccccc8oc7c6n(c6cccc1c6c32)c45] 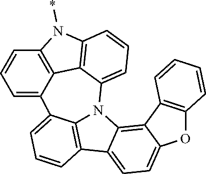 [SMILES: *n1c2cccc3c4cccc5c6ccc7oc8ccccc8c7c6n(c6cccc1c6c32)c45] 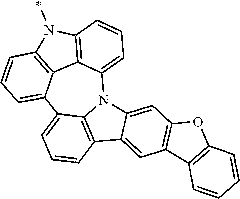 [SMILES: *n1c2cccc3c4cccc5c6cc7c(cc6n(c6cccc1c6c32)c45)oc1ccccc17] 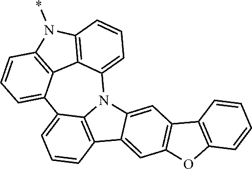 [SMILES: *n1c2cccc3c4cccc5c6cc7oc8ccccc8c7cc6n(c6cccc1c6c32)c45] 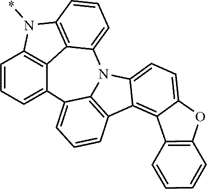 [SMILES: *n1c2cccc3c4cccc5c6c7c(ccc6n(c6cccc1c6c32)c45)oc1ccccc17] 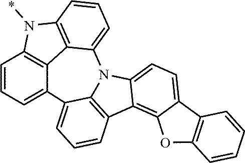 [SMILES: *n1c2cccc3c4cccc5c6c7oc8ccccc8c7ccc6n(c6cccc1c6c32)c45] 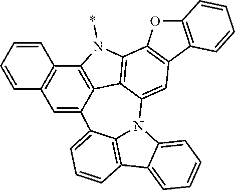 [SMILES: *n1c2c3ccccc3cc3c4cccc5c6ccccc6n(c6cc7c8ccccc8oc7c1c6c32)c45] 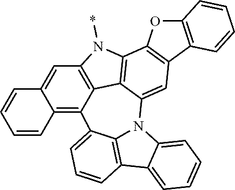 [SMILES: *n1c2cc3ccccc3c3c4cccc5c6ccccc6n(c6cc7c8ccccc8oc7c1c6c32)c54] 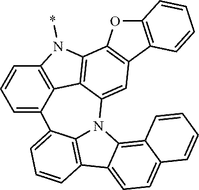 [SMILES: *n1c2cccc3c4cccc5c6ccc7ccccc7c6n(c6cc7c8ccccc8oc7c1c6c32)c45] 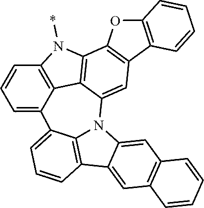 [SMILES: *n1c2cccc3c4cccc5c6cc7ccccc7cc6n(c6cc7c8ccccc8oc7c1c6c32)c45] 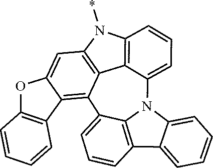 [SMILES: *n1c2cc3oc4ccccc4c3c3c4cccc5c6ccccc6n(c6cccc1c6c32)c54] 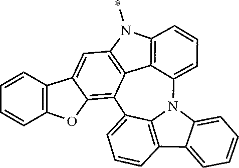 [SMILES: *n1c2cc3c4ccccc4oc3c3c4cccc5c6ccccc6n(c6cccc1c6c32)c54] 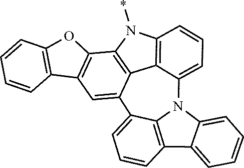 [SMILES: *n1c2cccc3c2c2c(cc4c5ccccc5oc4c21)c1cccc2c4ccccc4n3c12] 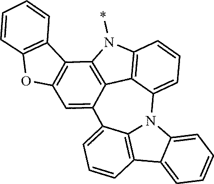 [SMILES: *n1c2cccc3c2c2c(cc4oc5ccccc5c4c21)c1cccc2c4ccccc4n3c12] 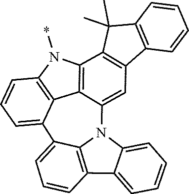 [SMILES: *n1c2cccc3c4cccc5c6ccccc6n(c6cc7c(c1c6c32)C(C)(C)c1ccccc1-7)c45] 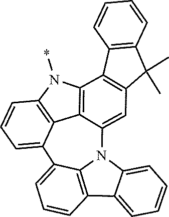 [SMILES: *n1c2cccc3c4cccc5c6ccccc6n(c6cc7c(c1c6c32)-c1ccccc1C7(C)C)c45] 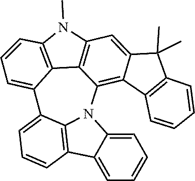 [SMILES: Cn1c2cccc3c4cccc5c6ccccc6n(c6c7c(cc1c6c32)C(C)(C)c1ccccc1-7)c45] 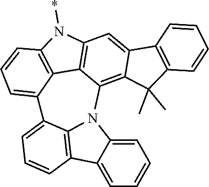 [SMILES: *n1c2cccc3c4cccc5c6ccccc6n(c6c7c(cc1c6c32)-c1ccccc1C7(C)C)c45] 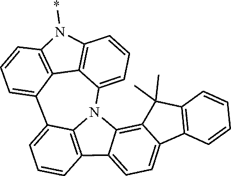 [SMILES: *n1c2cccc3c4cccc5c6ccc7c(c6n(c6cccc1c6c32)c45)C(C)(C)c1ccccc1-7] 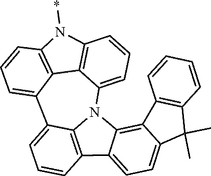 [SMILES: *n1c2cccc3c4cccc5c6ccc7c(c6n(c6cccc1c6c32)c45)-c1ccccc1C7(C)C] 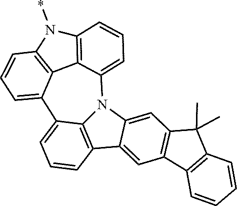 [SMILES: *n1c2cccc3c4cccc5c6cc7c(cc6n(c6cccc1c6c32)c45)C(C)(C)c1ccccc1-7] 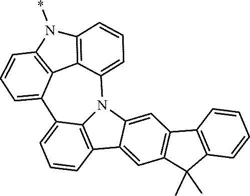 [SMILES: *n1c2cccc3c4cccc5c6cc7c(cc6n(c6cccc1c6c32)c45)-c1ccccc1C7(C)C] 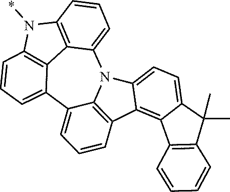 [SMILES: *n1c2cccc3c4cccc5c6c7c(ccc6n(c6cccc1c6c32)c45)C(C)(C)c1ccccc1-7] 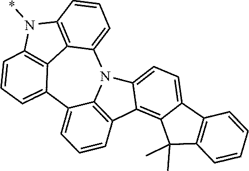 [SMILES: *n1c2cccc3c4cccc5c6c7c(ccc6n(c6cccc1c6c32)c45)-c1ccccc1C7(C)C] 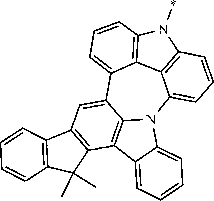 [SMILES: *n1c2cccc3c4cc5c(c6c7ccccc7n(c7cccc1c7c32)c46)C(C)(C)c1ccccc1-5] 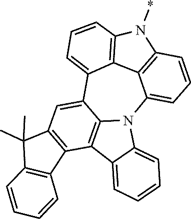 [SMILES: *n1c2cccc3c4cc5c(c6c7ccccc7n(c7cccc1c7c32)c46)-c1ccccc1C5(C)C] 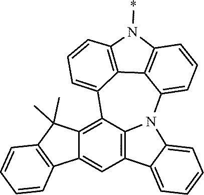 [SMILES: *n1c2cccc3c4c5c(cc6c7ccccc7n(c7cccc1c7c32)c64)-c1ccccc1C5(C)C] 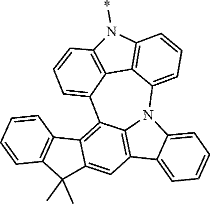 [SMILES: *n1c2cccc3c4c5c(cc6c7ccccc7n(c7cccc1c7c32)c64)C(C)(C)c1ccccc1-5] 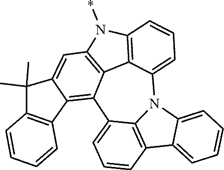 [SMILES: *n1c2cc3c(c4c5cccc6c7ccccc7n(c7cccc1c7c42)c65)-c1ccccc1C3(C)C] 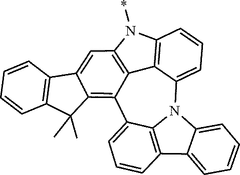 [SMILES: *n1c2cc3c(c4c5cccc6c7ccccc7n(c7cccc1c7c42)c65)C(C)(C)c1ccccc1-3] 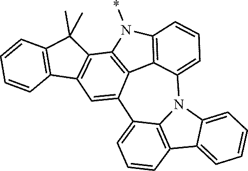 [SMILES: *n1c2cccc3c2c2c(cc4c(c21)C(C)(C)c1ccccc1-4)c1cccc2c4ccccc4n3c12] 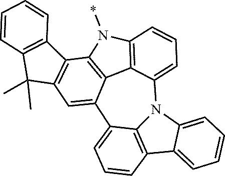 [SMILES: *n1c2cccc3c2c2c(cc4c(c21)-c1ccccc1C4(C)C)c1cccc2c4ccccc4n3c12] 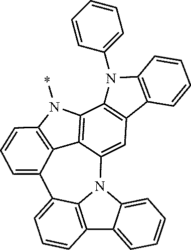 [SMILES: *n1c2cccc3c4cccc5c6ccccc6n(c6cc7c8ccccc8n(-c8ccccc8)c7c1c6c32)c45] 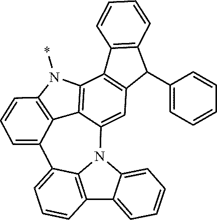 [SMILES: *n1c2cccc3c4cccc5c6ccccc6n(c6cc7c(c1c6c32)-c1ccccc1C7c1ccccc1)c45] 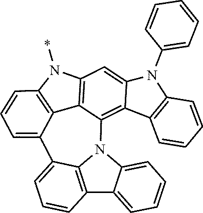 [SMILES: *n1c2cccc3c4cccc5c6ccccc6n(c45)c4c(c32)c1cc1c4c2ccccc2n1-c1ccccc1] 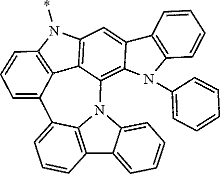 [SMILES: *n1c2cccc3c4cccc5c6ccccc6n(c45)c4c(c32)c1cc1c2ccccc2n(-c2ccccc2)c14] 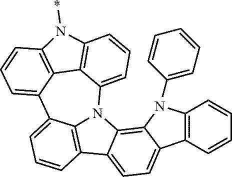 [SMILES: *n1c2cccc3c4cccc5c6ccc7c8ccccc8n(-c8ccccc8)c7c6n(c6cccc1c6c32)c45] 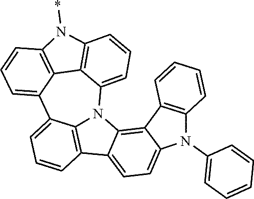 [SMILES: *n1c2cccc3c4cccc5c6ccc7c(c8ccccc8n7-c7ccccc7)c6n(c6cccc1c6c32)c45] 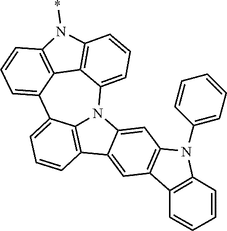 [SMILES: *n1c2cccc3c4cccc5c6cc7c8ccccc8n(-c8ccccc8)c7cc6n(c6cccc1c6c32)c45] 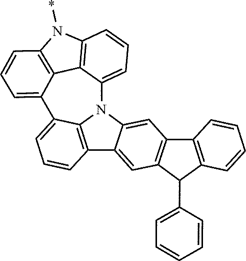 [SMILES: *n1c2cccc3c4cccc5c6cc7c(cc6n(c6cccc1c6c32)c45)-c1ccccc1C7c1ccccc1] 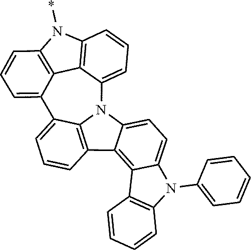 [SMILES: *n1c2cccc3c4cccc5c6c7c8ccccc8n(-c8ccccc8)c7ccc6n(c6cccc1c6c32)c45] 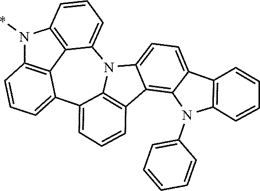 [SMILES: *n1c2cccc3c4cccc5c6c7c(ccc6n(c6cccc1c6c32)c45)c1ccccc1n7-c1ccccc1] 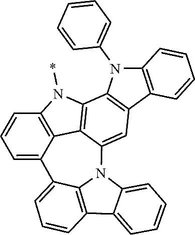 [SMILES: *n1c2cccc3c4cccc5c6ccccc6n(c6cc7c8ccccc8n(-c8ccccc8)c7c1c6c32)c45] 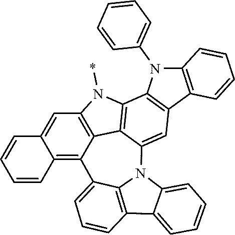 [SMILES: *n1c2cc3ccccc3c3c4cccc5c6ccccc6n(c6cc7c8ccccc8n(-c8ccccc8)c7c1c6c32)c54] 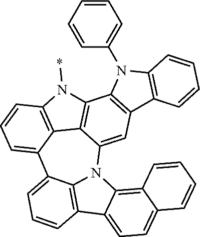 [SMILES: *n1c2cccc3c4cccc5c6ccc7ccccc7c6n(c6cc7c8ccccc8n(-c8ccccc8)c7c1c6c32)c45] 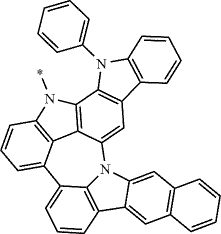 [SMILES: *n1c2cccc3c4cccc5c6cc7ccccc7cc6n(c6cc7c8ccccc8n(-c8ccccc8)c7c1c6c32)c45] 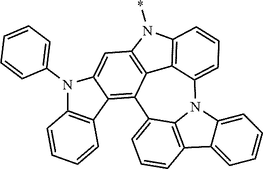 [SMILES: *n1c2cc3c(c4ccccc4n3-c3ccccc3)c3c4cccc5c6ccccc6n(c6cccc1c6c32)c54] 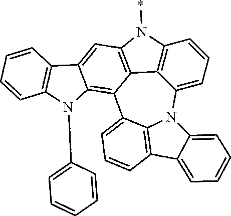 [SMILES: *n1c2cc3c4ccccc4n(-c4ccccc4)c3c3c4cccc5c6ccccc6n(c6cccc1c6c32)c54] 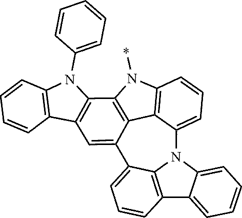 [SMILES: *n1c2cccc3c2c2c(cc4c5ccccc5n(-c5ccccc5)c4c21)c1cccc2c4ccccc4n3c12] 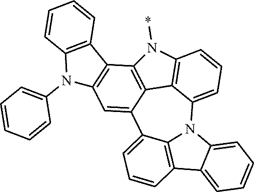 [SMILES: *n1c2cccc3c2c2c(cc4c(c5ccccc5n4-c4ccccc4)c21)c1cccc2c4ccccc4n3c12] 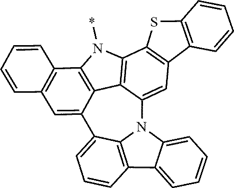 [SMILES: *n1c2c3ccccc3cc3c4cccc5c6ccccc6n(c6cc7c8ccccc8sc7c1c6c32)c45] 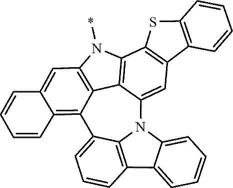 [SMILES: *n1c2cc3ccccc3c3c4cccc5c6ccccc6n(c6cc7c8ccccc8sc7c1c6c32)c54] 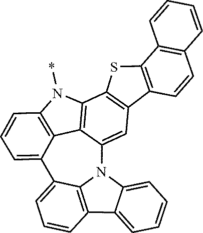 [SMILES: *n1c2cccc3c4cccc5c6ccccc6n(c6cc7c8ccc9ccccc9c8sc7c1c6c32)c45] 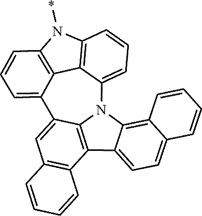 [SMILES: *n1c2cccc3c4cc5ccccc5c5c6ccc7ccccc7c6n(c6cccc1c6c32)c45] 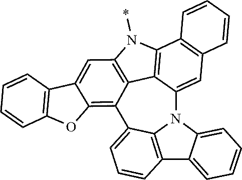 [SMILES: *n1c2cc3c4ccccc4oc3c3c4cccc5c6ccccc6n(c6cc7ccccc7c1c6c32)c54] 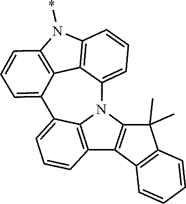 [SMILES: *n1c2cccc3c4cccc5c6c(n(c7cccc1c7c32)c54)C(C)(C)c1ccccc1-6] 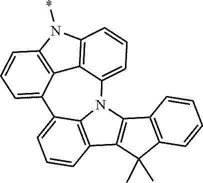 [SMILES: *n1c2cccc3c4cccc5c6c(n(c7cccc1c7c32)c54)-c1ccccc1C6(C)C] 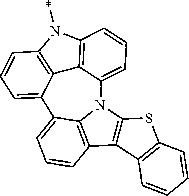 [SMILES: *n1c2cccc3c4cccc5c6c7ccccc7sc6n(c6cccc1c6c32)c45] 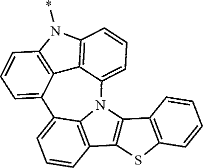 [SMILES: *n1c2cccc3c4cccc5c6sc7ccccc7c6n(c6cccc1c6c32)c45] 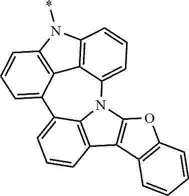 [SMILES: *n1c2cccc3c4cccc5c6c7ccccc7oc6n(c6cccc1c6c32)c45] 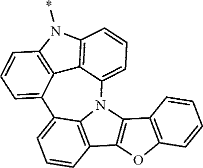 [SMILES: *n1c2cccc3c4cccc5c6oc7ccccc7c6n(c6cccc1c6c32)c45] 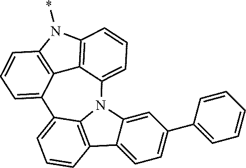 [SMILES: *n1c2cccc3c4cccc5c6ccc(-c7ccccc7)cc6n(c6cccc1c6c32)c45] 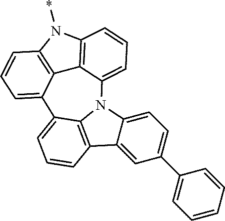 [SMILES: *n1c2cccc3c4cccc5c6cc(-c7ccccc7)ccc6n(c6cccc1c6c32)c45] 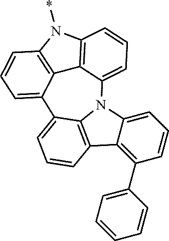 [SMILES: *n1c2cccc3c4cccc5c6c(-c7ccccc7)cccc6n(c6cccc1c6c32)c45] 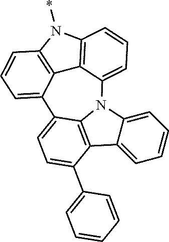 [SMILES: *n1c2cccc3c4ccc(-c5ccccc5)c5c6ccccc6n(c6cccc1c6c32)c45] 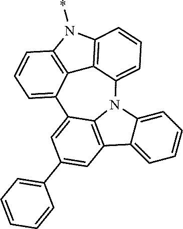 [SMILES: *n1c2cccc3c4cc(-c5ccccc5)cc5c6ccccc6n(c6cccc1c6c32)c45] 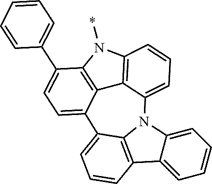 [SMILES: *n1c2cccc3c2c2c(ccc(-c4ccccc4)c21)c1cccc2c4ccccc4n3c12] 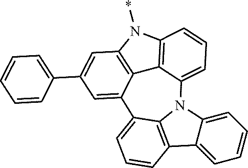 [SMILES: *n1c2cc(-c3ccccc3)cc3c4cccc5c6ccccc6n(c6cccc1c6c32)c45] 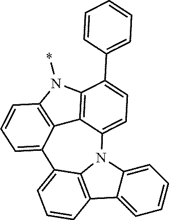 [SMILES: *n1c2cccc3c4cccc5c6ccccc6n(c6ccc(-c7ccccc7)c1c6c32)c45] 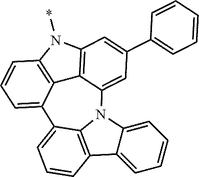 [SMILES: *n1c2cccc3c4cccc5c6ccccc6n(c6cc(-c7ccccc7)cc1c6c32)c45] 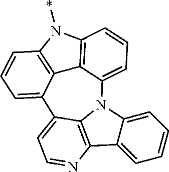 [SMILES: *n1c2cccc3c4ccnc5c6ccccc6n(c6cccc1c6c32)c45] 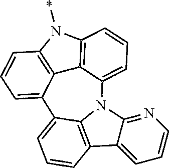 [SMILES: *n1c2cccc3c4cccc5c6cccnc6n(c6cccc1c6c32)c45] 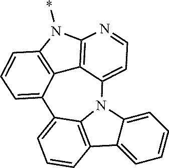 [SMILES: *n1c2cccc3c4cccc5c6ccccc6n(c6ccnc1c6c32)c45] 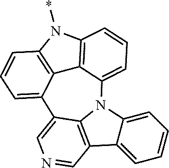 [SMILES: *n1c2cccc3c4cncc5c6ccccc6n(c6cccc1c6c32)c45] 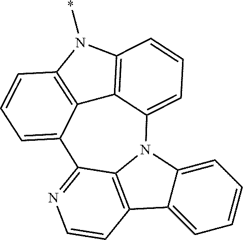 [SMILES: *n1c2cccc3c4nccc5c6ccccc6n(c6cccc1c6c32)c54] 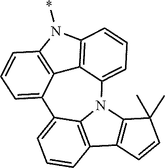 [SMILES: *n1c2cccc3c4cccc5c6c(n(c7cccc1c7c32)c54)C(C)(C)C=C6] 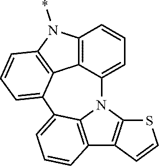 [SMILES: *n1c2cccc3c4cccc5c6ccsc6n(c6cccc1c6c32)c45] 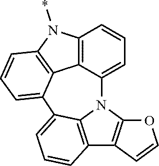 [SMILES: *n1c2cccc3c4cccc5c6ccoc6n(c6cccc1c6c32)c45] 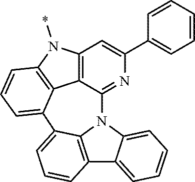 [SMILES: *n1c2cccc3c4cccc5c6ccccc6n(c6nc(-c7ccccc7)cc1c6c32)c45] 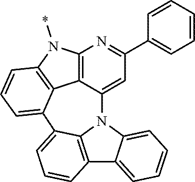 [SMILES: *n1c2cccc3c4cccc5c6ccccc6n(c6cc(-c7ccccc7)nc1c6c32)c45] 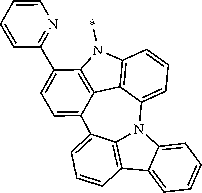 [SMILES: *n1c2cccc3c2c2c(ccc(-c4ccccn4)c21)c1cccc2c4ccccc4n3c12] 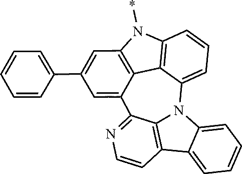 [SMILES: *n1c2cc(-c3ccccc3)cc3c4nccc5c6ccccc6n(c6cccc1c6c32)c54] 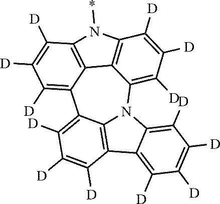 [SMILES: *n1c2c([2H])c([2H])c([2H])c3c4c([2H])c([2H])c([2H])c5c6c([2H])c([2H])c([2H])c([2H])c6n(c6c([2H])c([2H])c([2H])c1c6c32)c45] 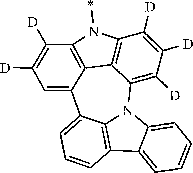 [SMILES: *n1c2c([2H])c([2H])cc3c4cccc5c6ccccc6n(c6c([2H])c([2H])c([2H])c1c6c32)c45] 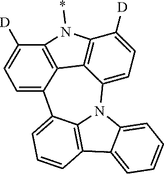 [SMILES: *n1c2c([2H])ccc3c4cccc5c6ccccc6n(c6ccc([2H])c1c6c32)c45] 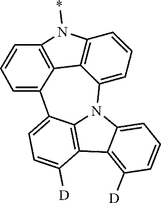 [SMILES: *n1c2cccc3c4ccc([2H])c5c6c([2H])cccc6n(c6cccc1c6c32)c45] 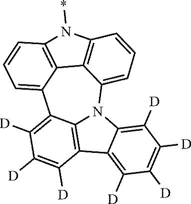 [SMILES: *n1c2cccc3c4c([2H])c([2H])c([2H])c5c6c([2H])c([2H])c([2H])c([2H])c6n(c6cccc1c6c32)c45] 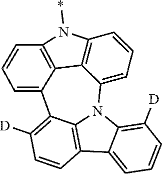 [SMILES: *n1c2cccc3c4c([2H])ccc5c6cccc([2H])c6n(c6cccc1c6c32)c54] 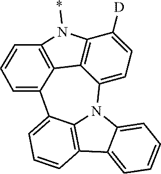 [SMILES: *n1c2cccc3c4cccc5c6ccccc6n(c6ccc([2H])c1c6c32)c45] 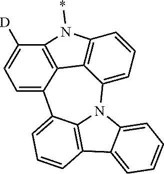 [SMILES: *n1c2cccc3c2c2c(ccc([2H])c21)c1cccc2c4ccccc4n3c12] 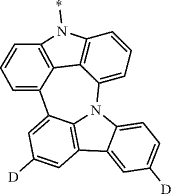 [SMILES: *n1c2cccc3c4cc([2H])cc5c6cc([2H])ccc6n(c6cccc1c6c32)c45] 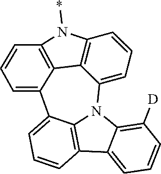 [SMILES: *n1c2cccc3c4cccc5c6cccc([2H])c6n(c6cccc1c6c32)c45] 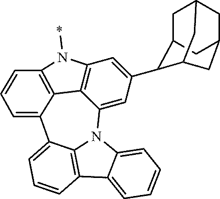 [SMILES: *n1c2cccc3c4cccc5c6ccccc6n(c6cc(C7C8CC9CC(C8)CC7C9)cc1c6c32)c45] 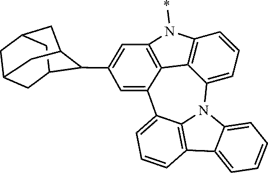 [SMILES: *n1c2cc(C3C4CC5CC(C4)CC3C5)cc3c4cccc5c6ccccc6n(c6cccc1c6c32)c45] 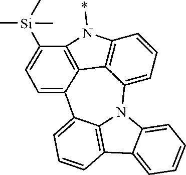 [SMILES: *n1c2cccc3c2c2c(ccc([Si](C)(C)C)c21)c1cccc2c4ccccc4n3c12] 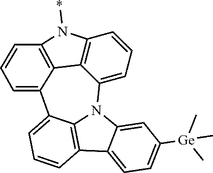 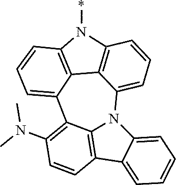 [SMILES: *n1c2cccc3c4c(N(C)C)ccc5c6ccccc6n(c6cccc1c6c32)c54] 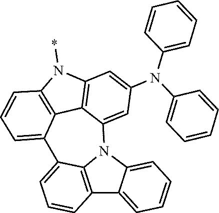 [SMILES: *n1c2cccc3c4cccc5c6ccccc6n(c6cc(N(c7ccccc7)c7ccccc7)cc1c6c32)c45] 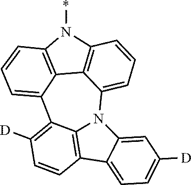 [SMILES: *n1c2cccc3c4c([2H])ccc5c6ccc([2H])cc6n(c6cccc1c6c32)c54]
wherein “^{∗}” represents a position where the structure of H-1 to H-139 is joined to L; and
optionally, hydrogens in the structures of H-1 to H-139 can be partially or fully substituted with deuterium.

5. The electroluminescent device according to claim 1, wherein E has a structure represented by Formula 1-a: 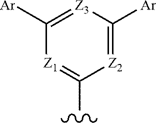
wherein Z₁ to Z₃ are each independently selected from N or CR_{z}, and at least two of Z₁ to Z₃ are N;
R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar is, at each occurrence identically or differently, selected from substituted or unsubstituted aryl having 6 to 18 carbon atoms or substituted or unsubstituted heteroaryl having 3 to 18 carbon atoms;
preferably, Z₁ to Z₃ are all N; and
more preferably, Ar is, at each occurrence identically or differently, selected from the group consisting of: phenyl, deuterated phenyl, methylphenyl, fluorophenyl, tert-butylphenyl, trideuteromethylphenyl, biphenyl, naphthyl, deuterated naphthyl, dibenzofuranyl, dibenzothienyl, 9,9-dimethylfluorenyl, carbazolyl, pyridyl, pyrimidinyl, 4-cyanophenyl, 3-cyanophenyl, triphenylenyl and combinations thereof.

6. The electroluminescent device according to claim 4, wherein E is selected from the group consisting of the following structures: 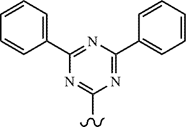 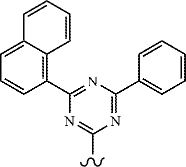 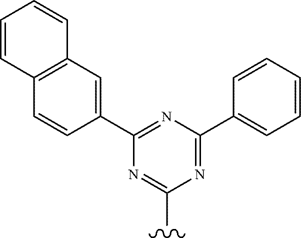 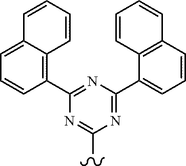 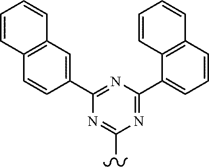 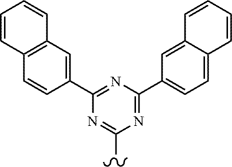 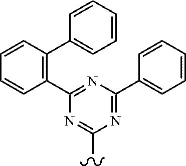 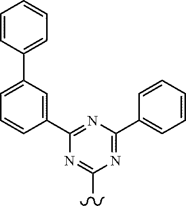 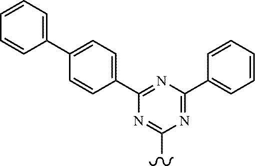 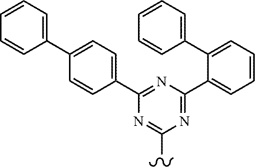 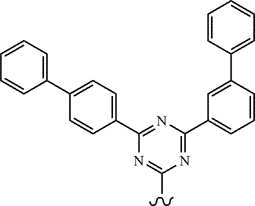 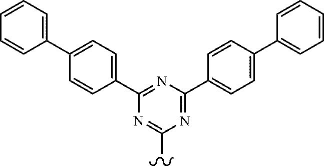 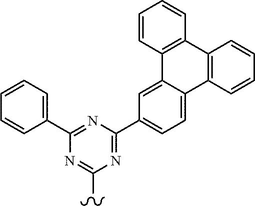 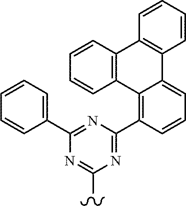 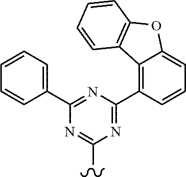 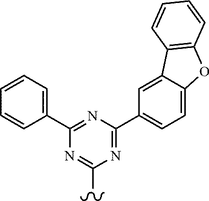 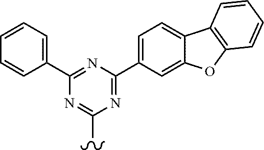 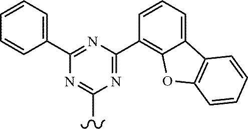 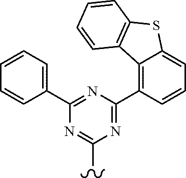 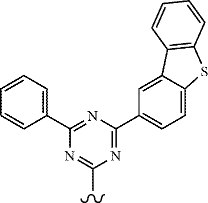 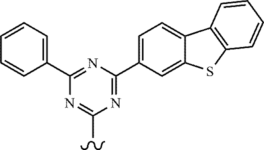 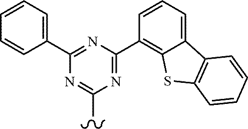 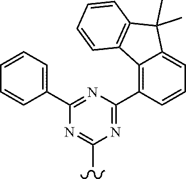 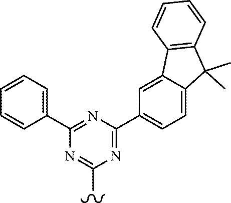 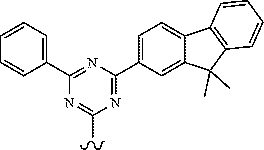 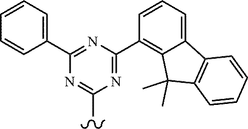 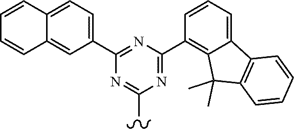 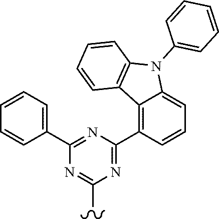 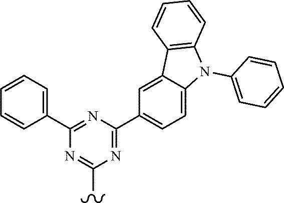 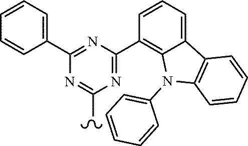 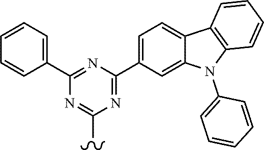 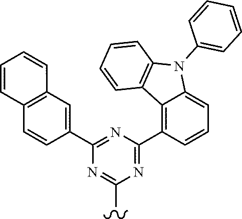 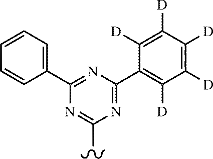 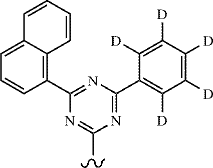 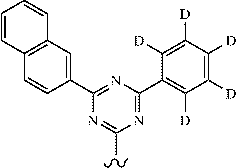 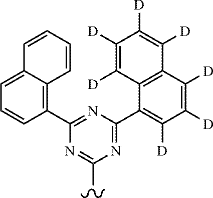 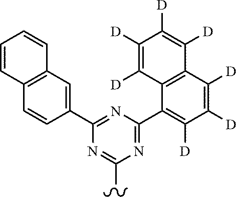 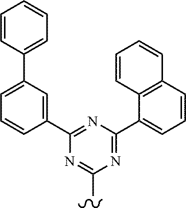 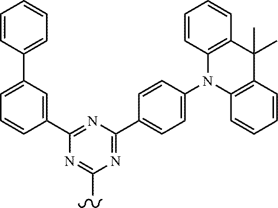 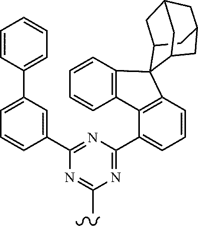 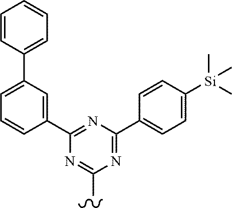 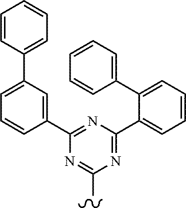 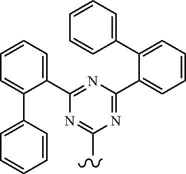 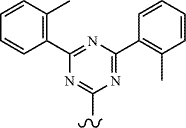 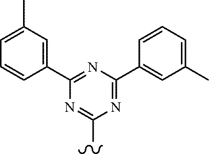 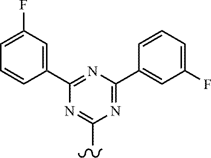 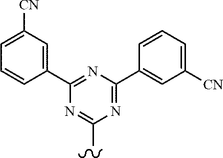 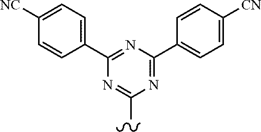 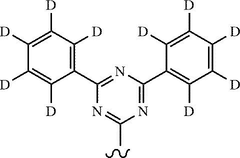 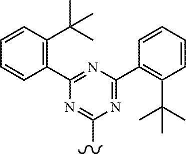 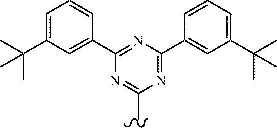 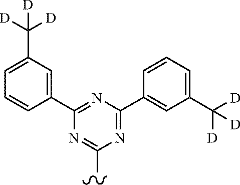 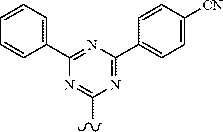 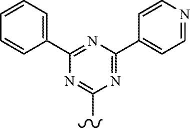 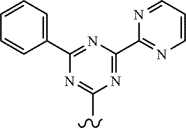 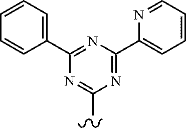 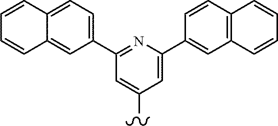 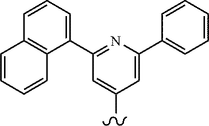 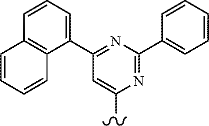 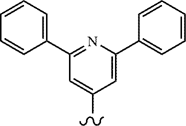 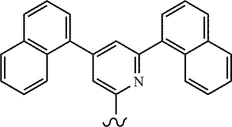 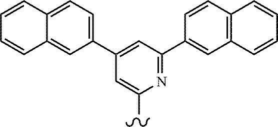 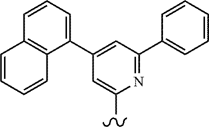 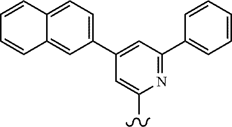 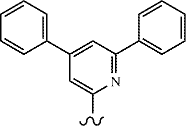 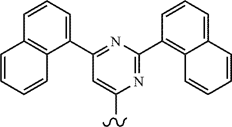 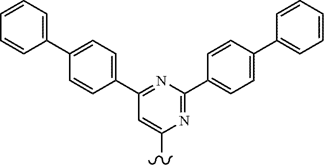 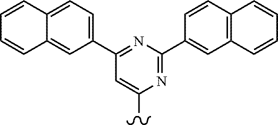 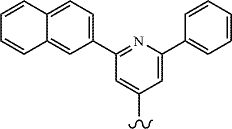 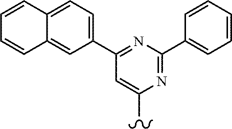 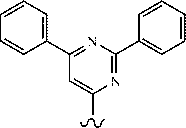 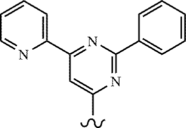 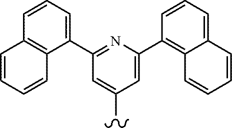 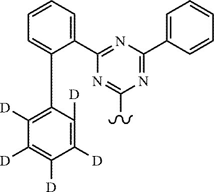 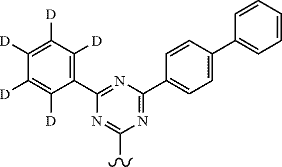 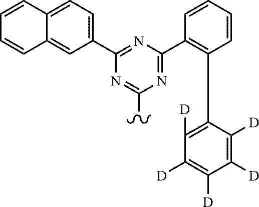 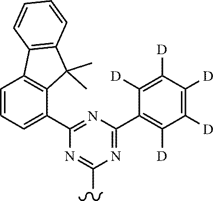 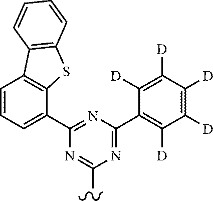 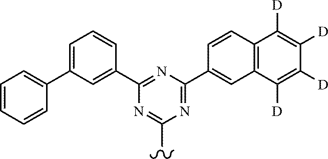 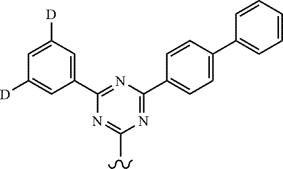 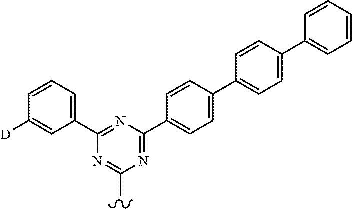 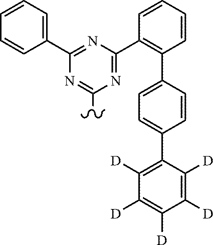 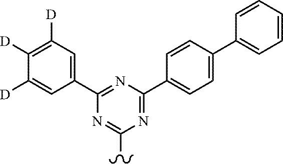 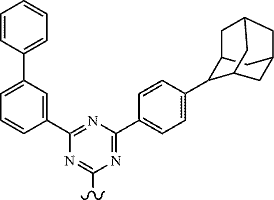 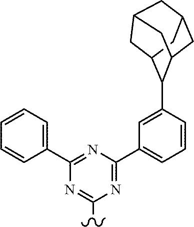 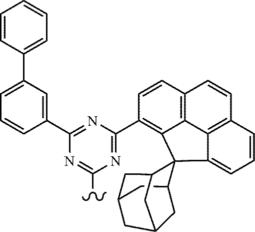 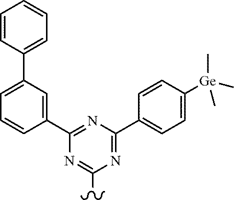 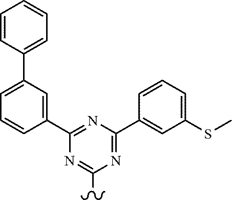 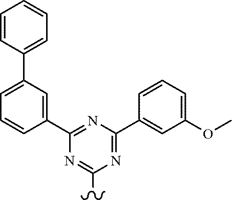 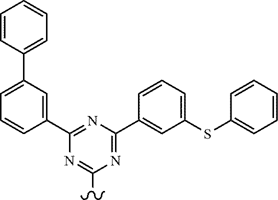 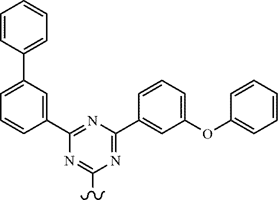 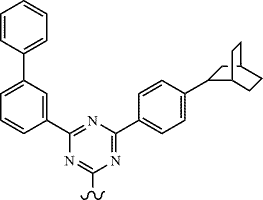 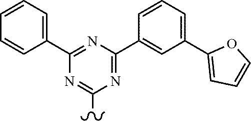 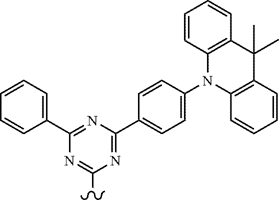 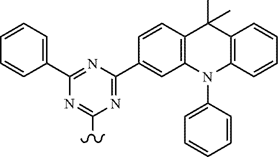
wherein optionally, hydrogens in the structures of E-1 to E-95 can be partially or fully substituted with deuterium.

7. The electroluminescent device according to claim 6, wherein L is selected from a single bond, substituted or unsubstituted arylene having 6 to 20 carbon atoms, substituted or unsubstituted heteroarylene having 3 to 20 carbon atoms or a combination thereof;
wherein preferably, L is selected from the group consisting of the following structures: 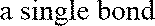 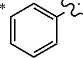 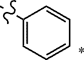 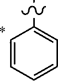 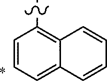 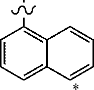 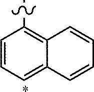 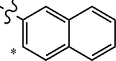 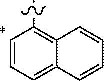 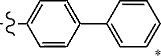 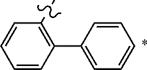 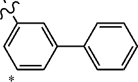 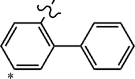 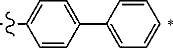 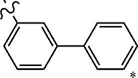 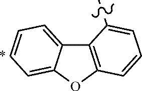 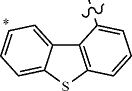 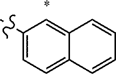 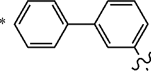 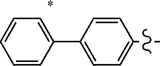 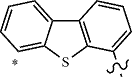 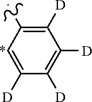 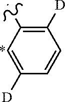 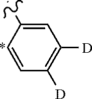 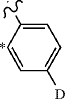 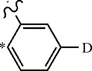 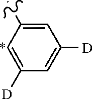 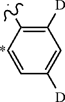
wherein “ 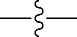
” represents a position where the structure of L-1 to L-27 is joined to E, and “^{∗}” represents a position where the structure of L-1 to L-27 is joined to H; and
optionally, hydrogens in the structures of L-1 to L-27 can be partially or fully substituted with deuterium.

8. The electroluminescent device according to claim 7, wherein the first compound has the structure of H-L-E, wherein H is selected from any one of the group consisting of H-1 to H-139, L is selected from any one of the group consisting of L-0 to L-27, and E is selected from any one of the group consisting of E-1 to E-95; optionally, hydrogens in the first compound can be partially or fully substituted with deuterium;
wherein preferably, the first compound is selected from the group consisting of Compound 1-1 to Compound 1-550, wherein Compound 1-1 to Compound 1-550 have the structure of H-L-E, wherein H, L and E correspond to structures shown in the following table, respectively:

| Compound No. | H | L | E | Compound No. | H | L | E |
|---|---|---|---|---|---|---|---|
| 1-1 | H-1 | L-0 | E-1 | 1-2 | H-1 | L-23 | E-1 |
| 1-3 | H-1 | L-0 | E-2 | 1-4 | H-1 | L-24 | E-1 |
| 1-5 | H-1 | L-0 | E-3 | 1-6 | H-1 | L-25 | E-1 |
| 1-7 | H-1 | L-0 | E-4 | 1-8 | H-1 | L-26 | E-1 |
| 1-9 | H-1 | L-0 | E-5 | 1-10 | H-1 | L-27 | E-1 |
| 1-11 | H-1 | L-0 | E-6 | 1-12 | H-1 | L-2 | E-2 |
| 1-13 | H-1 | L-0 | E-7 | 1-14 | H-1 | L-4 | E-2 |
| 1-15 | H-1 | L-0 | E-8 | 1-16 | H-1 | L-5 | E-2 |
| 1-17 | H-1 | L-0 | E-9 | 1-18 | H-1 | L-6 | E-2 |
| 1-19 | H-1 | L-0 | E-10 | 1-20 | H-1 | L-7 | E-2 |
| 1-21 | H-1 | L-0 | E-11 | 1-22 | H-1 | L-8 | E-2 |
| 1-23 | H-1 | L-0 | E-12 | 1-24 | H-1 | L-9 | E-2 |
| 1-25 | H-1 | L-0 | E-13 | 1-26 | H-1 | L-10 | E-2 |
| 1-27 | H-1 | L-0 | E-14 | 1-28 | H-1 | L-11 | E-2 |
| 1-29 | H-1 | L-0 | E-15 | 1-30 | H-1 | L-12 | E-2 |
| 1-31 | H-1 | L-0 | E-16 | 1-32 | H-1 | L-13 | E-2 |
| 1-33 | H-1 | L-0 | E-17 | 1-34 | H-1 | L-14 | E-2 |
| 1-35 | H-1 | L-0 | E-18 | 1-36 | H-1 | L-15 | E-2 |
| 1-37 | H-1 | L-0 | E-19 | 1-38 | H-1 | L-16 | E-2 |
| 1-39 | H-1 | L-0 | E-20 | 1-40 | H-1 | L-17 | E-2 |
| 1-41 | H-1 | L-0 | E-21 | 1-42 | H-1 | L-18 | E-2 |
| 1-43 | H-1 | L-0 | E-22 | 1–44 | H-1 | L-19 | E-2 |
| 1–45 | H-1 | L-0 | E-23 | 1–46 | H-1 | L-20 | E-2 |
| 1–47 | H-1 | L-0 | E-24 | 1–48 | H-1 | L-21 | E-2 |
| 1–49 | H-1 | L-0 | E-25 | 1–50 | H-1 | L-22 | E-2 |
| 1–51 | H-1 | L-0 | E-26 | 1–52 | H-1 | L-23 | E-2 |
| 1–53 | H-1 | L-0 | E-27 | 1–54 | H-1 | L-24 | E-2 |
| 1–55 | H-1 | L-0 | E-28 | 1–56 | H-1 | L-25 | E-2 |
| 1–57 | H-1 | L-0 | E-29 | 1–58 | H-1 | L-26 | E-2 |
| 1–59 | H-1 | L-0 | E-30 | 1–60 | H-1 | L-27 | E-2 |
| 1–61 | H-1 | L-0 | E-31 | 1–62 | H-1 | L-2 | E-3 |
| 1–63 | H-1 | L-0 | E-32 | 1–64 | H-1 | L-4 | E-3 |
| 1–65 | H-1 | L-0 | E-33 | 1–66 | H-1 | L-5 | E-3 |
| 1–67 | H-1 | L-0 | E-34 | 1–68 | H-1 | L-6 | E-3 |
| 1–69 | H-1 | L-0 | E-35 | 1–70 | H-1 | L-7 | E-3 |
| 1–71 | H-1 | L-0 | E-36 | 1–72 | H-1 | L-8 | E-3 |
| 1–73 | H-1 | L-0 | E-37 | 1–74 | H-1 | L-9 | E-3 |
| 1–75 | H-1 | L-0 | E-38 | 1–76 | H-1 | L-10 | E-3 |
| 1–77 | H-1 | L-0 | E-39 | 1–78 | H-1 | L-11 | E-3 |
| 1–79 | H-1 | L-0 | E-40 | 1–80 | H-1 | L-12 | E-3 |
| 1–81 | H-1 | L-0 | E-41 | 1–82 | H-1 | L-13 | E-3 |
| 1–83 | H-1 | L-0 | E-42 | 1–84 | H-1 | L-14 | E-3 |
| 1–85 | H-1 | L-0 | E-43 | 1–86 | H-1 | L-15 | E-3 |
| 1–87 | H-1 | L-0 | E-44 | 1–88 | H-1 | L-16 | E-3 |
| 1–89 | H-1 | L-0 | E-45 | 1–90 | H-1 | L-17 | E-3 |
| 1–91 | H-1 | L-0 | E-46 | 1–92 | H-1 | L-18 | E-3 |
| 1–93 | H-1 | L-0 | E-47 | 1–94 | H-1 | L-19 | E-3 |
| 1–95 | H-1 | L-0 | E-48 | 1–96 | H-1 | L-20 | E-3 |
| 1–97 | H-1 | L-0 | E-49 | 1–98 | H-1 | L-21 | E-3 |
| 1–99 | H-1 | L-0 | E-50 | 1–100 | H-1 | L-22 | E-3 |
| 1–101 | H-1 | L-0 | E-51 | 1–102 | H-1 | L-23 | E-3 |
| 1–103 | H-1 | L-0 | E-52 | 1–104 | H-1 | L-24 | E-3 |
| 1–105 | H-1 | L-0 | E-53 | 1–106 | H-1 | L-25 | E-3 |
| 1–107 | H-1 | L-0 | E-54 | 1–108 | H-1 | L-26 | E-3 |
| 1–109 | H-1 | L-0 | E-55 | 1–110 | H-1 | L-27 | E-3 |
| 1–111 | H-1 | L-0 | E-56 | 1–112 | H-2 | L-2 | E-4 |
| 1–113 | H-1 | L-0 | E-57 | 1–114 | H-3 | L-4 | E-5 |
| 1–115 | H-1 | L-0 | E-58 | 1–116 | H-4 | L-5 | E-6 |
| 1–117 | H-1 | L-0 | E-59 | 1–118 | H-5 | L-6 | E-7 |
| 1–119 | H-1 | L-0 | E-60 | 1–120 | H-6 | L-7 | E-8 |
| 1–121 | H-1 | L-0 | E-61 | 1–122 | H-7 | L-8 | E-9 |
| 1–123 | H-1 | L-0 | E-62 | 1–124 | H-8 | L-9 | E-10 |
| 1–125 | H-1 | L-0 | E-63 | 1–126 | H-9 | L-10 | E-11 |
| 1–127 | H-1 | L-0 | E-64 | 1–128 | H-10 | L-11 | E-12 |
| 1–129 | H-1 | L-0 | E-65 | 1–130 | H-11 | L-12 | E-13 |
| 1–131 | H-1 | L-0 | E-66 | 1–132 | H-12 | L-13 | E-14 |
| 1–133 | H-1 | L-0 | E-67 | 1–134 | H-13 | L-14 | E-15 |
| 1–135 | H-1 | L-0 | E-68 | 1–136 | H-14 | L-15 | E-16 |
| 1–137 | H-1 | L-0 | E-69 | 1–138 | H-15 | L-16 | E-17 |
| 1–139 | H-1 | L-0 | E-70 | 1–140 | H-16 | L-17 | E-18 |
| 1–141 | H-1 | L-0 | E-71 | 1–142 | H-17 | L-18 | E-19 |
| 1–143 | H-1 | L-0 | E-72 | 1–144 | H-18 | L-19 | E-20 |
| 1–145 | H-1 | L-0 | E-73 | 1–146 | H-19 | L-20 | E-21 |
| 1–147 | H-1 | L-0 | E-74 | 1–148 | H-20 | L-21 | E-22 |
| 1–149 | H-1 | L-0 | E-75 | 1–150 | H-21 | L-22 | E-23 |
| 1–151 | H-1 | L-0 | E-76 | 1–152 | H-22 | L-23 | E-24 |
| 1–153 | H-1 | L-0 | E-77 | 1–154 | H-23 | L-24 | E-25 |
| 1–155 | H-1 | L-0 | E-78 | 1–156 | H-24 | L-25 | E-26 |
| 1–157 | H-1 | L-0 | E-79 | 1–158 | H-25 | L-26 | E-27 |
| 1–159 | H-1 | L-0 | E-80 | 1–160 | H-26 | L-27 | E-28 |
| 1–161 | H-1 | L-0 | E-81 | 1–162 | H-27 | L-2 | E-29 |
| 1–163 | H-1 | L-0 | E-82 | 1–164 | H-28 | L-4 | E-30 |
| 1–165 | H-1 | L-0 | E-83 | 1–166 | H-29 | L-5 | E-31 |
| 1–167 | H-1 | L-1 | E-1 | 1–168 | H-30 | L-6 | E-32 |
| 1–169 | H-1 | L-1 | E-2 | 1–170 | H-31 | L-7 | E-33 |
| 1–171 | H-1 | L-1 | E-3 | 1–172 | H-32 | L-8 | E-34 |
| 1–173 | H-1 | L-1 | E-4 | 1–174 | H-33 | L-9 | E-35 |
| 1–175 | H-1 | L-1 | E-5 | 1–176 | H-34 | L-10 | E-36 |
| 1–177 | H-1 | L-1 | E-6 | 1–178 | H-35 | L-11 | E-37 |
| 1–179 | H-1 | L-1 | E-7 | 1–180 | H-36 | L-12 | E-38 |
| 1–181 | H-1 | L-1 | E-8 | 1–182 | H-37 | L-13 | E-39 |
| 1–183 | H-1 | L-1 | E-9 | 1–184 | H-38 | L-14 | E-40 |
| 1–185 | H-1 | L-1 | E-10 | 1–186 | H-39 | L-15 | E-41 |
| 1–187 | H-1 | L-1 | E-11 | 1–188 | H-40 | L-16 | E-42 |
| 1–189 | H-1 | L-1 | E-12 | 1–190 | H-41 | L-17 | E-43 |
| 1–191 | H-1 | L-1 | E-13 | 1–192 | H-42 | L-18 | E-44 |
| 1–193 | H-1 | L-1 | E-14 | 1–194 | H-43 | L-19 | E-45 |
| 1–195 | H-1 | L-1 | E-15 | 1–196 | H-44 | L-20 | E-46 |
| 1–197 | H-1 | L-1 | E-16 | 1–198 | H-45 | L-21 | E-47 |
| 1–199 | H-1 | L-1 | E-17 | 1–200 | H-46 | L-22 | E-48 |
| 1–201 | H-1 | L-1 | E-18 | 1–202 | H-47 | L-23 | E-49 |
| 1–203 | H-1 | L-1 | E-19 | 1–204 | H-48 | L-24 | E-50 |
| 1–205 | H-1 | L-1 | E-20 | 1–206 | H-49 | L-25 | E-51 |
| 1–207 | H-1 | L-1 | E-21 | 1–208 | H-50 | L-26 | E-52 |
| 1–209 | H-1 | L-1 | E-22 | 1–210 | H-51 | L-27 | E-53 |
| 1–211 | H-1 | L-1 | E-23 | 1–212 | H-52 | L-2 | E-54 |
| 1–213 | H-1 | L-1 | E-24 | 1–214 | H-53 | L-4 | E-55 |
| 1–215 | H-1 | L-1 | E-25 | 1–216 | H-54 | L-5 | E-56 |
| 1–217 | H-1 | L-1 | E-26 | 1–218 | H-55 | L-6 | E-57 |
| 1–219 | H-1 | L-1 | E-27 | 1–220 | H-56 | L-7 | E-58 |
| 1–221 | H-1 | L-1 | E-28 | 1–222 | H-57 | L-8 | E-59 |
| 1–223 | H-1 | L-1 | E-29 | 1–224 | H-58 | L-9 | E-60 |
| 1–225 | H-1 | L-1 | E-30 | 1–226 | H-59 | L-10 | E-61 |
| 1–227 | H-1 | L-1 | E-31 | 1–228 | H-60 | L-11 | E-62 |
| 1–229 | H-1 | L-1 | E-32 | 1–230 | H-61 | L-12 | E-63 |
| 1–231 | H-1 | L-1 | E-33 | 1–232 | H-62 | L-13 | E-64 |
| 1–233 | H-1 | L-1 | E-34 | 1–234 | H-63 | L-14 | E-65 |
| 1–235 | H-1 | L-1 | E-35 | 1–236 | H-64 | L-15 | E-66 |
| 1–237 | H-1 | L-1 | E-36 | 1–238 | H-65 | L-16 | E-67 |
| 1–239 | H-1 | L-1 | E-37 | 1–240 | H-66 | L-17 | E-68 |
| 1–241 | H-1 | L-1 | E-38 | 1–242 | H-67 | L-18 | E-69 |
| 1–243 | H-1 | L-1 | E-39 | 1–244 | H-68 | L-19 | E-70 |
| 1–245 | H-1 | L-1 | E-40 | 1–246 | H-69 | L-20 | E-71 |
| 1–247 | H-1 | L-1 | E-41 | 1–248 | H-70 | L-21 | E-72 |
| 1–249 | H-1 | L-1 | E-42 | 1–250 | H-71 | L-22 | E-73 |
| 1–251 | H-1 | L-1 | E-43 | 1–252 | H-72 | L-23 | E-74 |
| 1–253 | H-1 | L-1 | E-44 | 1–254 | H-73 | L-24 | E-75 |
| 1–255 | H-1 | L-1 | E-45 | 1–256 | H-74 | L-25 | E-76 |
| 1–257 | H-1 | L-1 | E-46 | 1–258 | H-75 | L-26 | E-77 |
| 1–259 | H-1 | L-1 | E-47 | 1–260 | H-76 | L-27 | E-78 |
| 1–261 | H-1 | L-1 | E-48 | 1–262 | H-2 | L-3 | E-79 |
| 1–263 | H-1 | L-1 | E-49 | 1–264 | H-3 | L-3 | E-80 |
| 1–265 | H-1 | L-1 | E-50 | 1–266 | H-4 | L-3 | E-81 |
| 1–267 | H-1 | L-1 | E-51 | 1–268 | H-5 | L-3 | E-82 |
| 1–269 | H-1 | L-1 | E-52 | 1–270 | H-6 | L-3 | E-83 |
| 1–271 | H-1 | L-1 | E-53 | 1–272 | H-7 | L-3 | E-1 |
| 1–273 | H-1 | L-1 | E-54 | 1–274 | H-8 | L-3 | E-1 |
| 1–275 | H-1 | L-1 | E-55 | 1–276 | H-9 | L-3 | E-1 |
| 1–277 | H-1 | L-1 | E-56 | 1–278 | H-10 | L-3 | E-1 |
| 1–279 | H-1 | L-1 | E-57 | 1–280 | H-11 | L-3 | E-1 |
| 1–281 | H-1 | L-1 | E-58 | 1–282 | H-12 | L-3 | E-1 |
| 1–283 | H-1 | L-1 | E-59 | 1–284 | H-13 | L-3 | E-1 |
| 1–285 | H-1 | L-1 | E-60 | 1–286 | H-14 | L-3 | E-1 |
| 1–287 | H-1 | L-1 | E-61 | 1–288 | H-15 | L-3 | E-1 |
| 1–289 | H-1 | L-1 | E-62 | 1–290 | H-16 | L-3 | E-1 |
| 1–291 | H-1 | L-1 | E-63 | 1–292 | H-17 | L-3 | E-1 |
| 1–293 | H-1 | L-1 | E-64 | 1–294 | H-18 | L-3 | E-1 |
| 1–295 | H-1 | L-1 | E-65 | 1–296 | H-19 | L-3 | E-1 |
| 1–297 | H-1 | L-1 | E-66 | 1–298 | H-20 | L-3 | E-1 |
| 1–299 | H-1 | L-1 | E-67 | 1–300 | H-21 | L-3 | E-1 |
| 1–301 | H-1 | L-1 | E-68 | 1–302 | H-22 | L-3 | E-1 |
| 1–303 | H-1 | L-1 | E-69 | 1–304 | H-23 | L-3 | E-1 |
| 1–305 | H-1 | L-1 | E-70 | 1–306 | H-24 | L-3 | E-1 |
| 1-307 | H-1 | L-1 | E-71 | 1-308 | H-25 | L-3 | E-1 |
| 1-309 | H-1 | L-1 | E-72 | 1-310 | H-26 | L-3 | E-1 |
| 1-311 | H-1 | L-1 | E-73 | 1-312 | H-27 | L-3 | E-1 |
| 1-313 | H-1 | L-1 | E-74 | 1-314 | H-28 | L-3 | E-1 |
| 1-315 | H-1 | L-1 | E-75 | 1-316 | H-29 | L-3 | E-1 |
| 1-317 | H-1 | L-1 | E-76 | 1-318 | H-30 | L-3 | E-1 |
| 1-319 | H-1 | L-1 | E-77 | 1-320 | H-31 | L-3 | E-1 |
| 1-321 | H-1 | L-1 | E-78 | 1-322 | H-32 | L-3 | E-1 |
| 1-323 | H-1 | L-1 | E-79 | 1-324 | H-33 | L-3 | E-1 |
| 1-325 | H-1 | L-1 | E-80 | 1-326 | H-34 | L-3 | E-1 |
| 1-327 | H-1 | L-1 | E-81 | 1-328 | H-35 | L-3 | E-1 |
| 1-329 | H-1 | L-1 | E-82 | 1-330 | H-36 | L-3 | E-1 |
| 1-331 | H-1 | L-3 | E-83 | 1-323 | H-37 | L-3 | E-1 |
| 1-333 | H-1 | L-3 | E-1 | 1-334 | H-38 | L-3 | E-1 |
| 1-335 | H-1 | L-3 | E-2 | 1-336 | H-39 | L-3 | E-1 |
| 1-337 | H-1 | L-3 | E-3 | 1-338 | H-40 | L-3 | E-1 |
| 1-339 | H-1 | L-3 | E-4 | 1-340 | H-41 | L-3 | E-1 |
| 1-341 | H-1 | L-3 | E-5 | 1-342 | H-42 | L-3 | E-1 |
| 1-343 | H-1 | L-3 | E-6 | 1-344 | H-43 | L-3 | E-1 |
| 1-345 | H-1 | L-3 | E-7 | 1-346 | H-44 | L-3 | E-1 |
| 1-347 | H-1 | L-3 | E-8 | 1-348 | H-45 | L-3 | E-1 |
| 1-349 | H-1 | L-3 | E-9 | 1-350 | H-46 | L-3 | E-1 |
| 1-351 | H-1 | L-3 | E-10 | 1-352 | H-47 | L-3 | E-1 |
| 1-353 | H-1 | L-3 | E-11 | 1-354 | H-48 | L-3 | E-1 |
| 1-355 | H-1 | L-3 | E-12 | 1-356 | H-49 | L-3 | E-1 |
| 1-357 | H-1 | L-3 | E-13 | 1-358 | H-50 | L-3 | E-1 |
| 1-359 | H-1 | L-3 | E-14 | 1-360 | H-51 | L-3 | E-1 |
| 1-361 | H-1 | L-3 | E-15 | 1-362 | H-52 | L-3 | E-1 |
| 1-363 | H-1 | L-3 | E-16 | 1-364 | H-53 | L-3 | E-1 |
| 1-365 | H-1 | L-3 | E-17 | 1-366 | H-54 | L-3 | E-1 |
| 1-367 | H-1 | L-3 | E-18 | 1-368 | H-55 | L-3 | E-1 |
| 1-369 | H-1 | L-3 | E-19 | 1-370 | H-56 | L-3 | E-1 |
| 1-371 | H-1 | L-3 | E-20 | 1-372 | H-57 | L-3 | E-1 |
| 1-373 | H-1 | L-3 | E-21 | 1-374 | H-58 | L-3 | E-1 |
| 1-375 | H-1 | L-3 | E-22 | 1-376 | H-59 | L-3 | E-1 |
| 1-377 | H-1 | L-3 | E-23 | 1-378 | H-60 | L-3 | E-1 |
| 1-379 | H-1 | L-3 | E-24 | 1-380 | H-61 | L-3 | E-1 |
| 1-381 | H-1 | L-3 | E-25 | 1-382 | H-62 | L-3 | E-1 |
| 1-383 | H-1 | L-3 | E-26 | 1-384 | H-63 | L-3 | E-1 |
| 1-385 | H-1 | L-3 | E-27 | 1-386 | H-64 | L-3 | E-1 |
| 1-387 | H-1 | L-3 | E-28 | 1-388 | H-65 | L-3 | E-1 |
| 1-389 | H-1 | L-3 | E-29 | 1-390 | H-66 | L-3 | E-1 |
| 1-391 | H-1 | L-3 | E-30 | 1-392 | H-67 | L-3 | E-1 |
| 1-393 | H-1 | L-3 | E-31 | 1-394 | H-68 | L-3 | E-1 |
| 1-395 | H-1 | L-3 | E-32 | 1–396 | H-69 | L-3 | E-1 |
| 1-397 | H-1 | L-3 | E-33 | 1–398 | H-70 | L-3 | E-1 |
| 1-399 | H-1 | L-3 | E-34 | 1–400 | H-71 | L-3 | E-1 |
| 1–401 | H-1 | L-3 | E-35 | 1–402 | H-72 | L-3 | E-1 |
| 1–403 | H-1 | L-3 | E-36 | 1–404 | H-73 | L-3 | E-1 |
| 1–405 | H-1 | L-3 | E-37 | 1–406 | H-74 | L-3 | E-1 |
| 1–407 | H-1 | L-3 | E-38 | 1–408 | H-75 | L-3 | E-1 |
| 1–409 | H-1 | L-3 | E-39 | 1–411 | H-76 | L-3 | E-1 |
| 1–411 | H-1 | L-3 | E-40 | 1–412 | H-77 | L-3 | E-1 |
| 1–413 | H-1 | L-3 | E-41 | 1–414 | H-78 | L-3 | E-1 |
| 1–415 | H-1 | L-3 | E-42 | 1–416 | H-79 | L-3 | E-1 |
| 1–417 | H-1 | L-3 | E-43 | 1–418 | H-80 | L-3 | E-1 |
| 1–419 | H-1 | L-3 | E-44 | 1–420 | H-81 | L-3 | E-1 |
| 1–421 | H-1 | L-3 | E-45 | 1–422 | H-82 | L-3 | E-1 |
| 1–423 | H-1 | L-3 | E-46 | 1–424 | H-83 | L-3 | E-1 |
| 1–425 | H-1 | L-3 | E-47 | 1–426 | H-84 | L-3 | E-1 |
| 1–427 | H-1 | L-3 | E-48 | 1–428 | H-85 | L-3 | E-1 |
| 1–429 | H-1 | L-3 | E-49 | 1–430 | H-86 | L-3 | E-1 |
| 1–431 | H-1 | L-3 | E-50 | 1–432 | H-87 | L-3 | E-1 |
| 1–433 | H-1 | L-3 | E-51 | 1–434 | H-88 | L-3 | E-1 |
| 1–435 | H-1 | L-3 | E-52 | 1–436 | H-89 | L-3 | E-1 |
| 1–437 | H-1 | L-3 | E-53 | 1–438 | H-90 | L-3 | E-1 |
| 1–439 | H-1 | L-3 | E-54 | 1–440 | H-91 | L-3 | E-1 |
| 1–441 | H-1 | L-3 | E-55 | 1–442 | H-92 | L-3 | E-1 |
| 1–443 | H-1 | L-3 | E-56 | 1–444 | H-93 | L-3 | E-1 |
| 1–445 | H-1 | L-3 | E-57 | 1–446 | H-94 | L-3 | E-1 |
| 1–447 | H-1 | L-3 | E-58 | 1–448 | H-95 | L-3 | E-1 |
| 1–449 | H-1 | L-3 | E-59 | 1–450 | H-96 | L-3 | E-1 |
| 1–451 | H-1 | L-3 | E-60 | 1–452 | H-97 | L-3 | E-1 |
| 1–453 | H-1 | L-3 | E-61 | 1–454 | H-98 | L-3 | E-1 |
| 1–455 | H-1 | L-3 | E-62 | 1–456 | H-99 | L-3 | E-1 |
| 1–457 | H-1 | L-3 | E-63 | 1–458 | H-100 | L-3 | E-1 |
| 1–459 | H-1 | L-3 | E-64 | 1–460 | H-101 | L-3 | E-1 |
| 1–461 | H-1 | L-3 | E-65 | 1–462 | H-102 | L-3 | E-1 |
| 1–463 | H-1 | L-3 | E-66 | 1–464 | H-103 | L-3 | E-1 |
| 1–465 | H-1 | L-3 | E-67 | 1–466 | H-104 | L-3 | E-1 |
| 1–467 | H-1 | L-3 | E-68 | 1–468 | H-105 | L-3 | E-1 |
| 1–469 | H-1 | L-3 | E-69 | 1–470 | H-106 | L-3 | E-1 |
| 1–471 | H-1 | L-3 | E-70 | 1–472 | H-107 | L-3 | E-1 |
| 1–473 | H-1 | L-3 | E-71 | 1–474 | H-108 | L-3 | E-1 |
| 1–475 | H-1 | L-3 | E-72 | 1–476 | H-109 | L-3 | E-1 |
| 1–477 | H-1 | L-3 | E-73 | 1–478 | H-110 | L-3 | E-1 |
| 1–479 | H-1 | L-3 | E-74 | 1–480 | H-111 | L-3 | E-1 |
| 1–481 | H-1 | L-3 | E-75 | 1–482 | H-112 | L-3 | E-1 |
| 1–483 | H-1 | L-3 | E-76 | 1–484 | H-113 | L-3 | E-1 |
| 1–485 | H-1 | L-3 | E-77 | 1–486 | H-114 | L-3 | E-1 |
| 1–487 | H-1 | L-3 | E-78 | 1–488 | H-115 | L-3 | E-1 |
| 1–489 | H-1 | L-3 | E-79 | 1–490 | H-116 | L-3 | E-1 |
| 1–491 | H-1 | L-3 | E-80 | 1–492 | H-117 | L-3 | E-1 |
| 1–493 | H-1 | L-3 | E-81 | 1–494 | H-118 | L-3 | E-1 |
| 1–495 | H-1 | L-3 | E-82 | 1–496 | H-119 | L-3 | E-1 |
| 1–497 | H-1 | L-3 | E-83 | 1–498 | H-120 | L-3 | E-1 |
| 1–499 | H-1 | L-2 | E-1 | 1–500 | H-121 | L-3 | E-1 |
| 1–501 | H-1 | L-4 | E-1 | 1–502 | H-122 | L-3 | E-1 |
| 1–503 | H-1 | L-5 | E-1 | 1–504 | H-123 | L-3 | E-1 |
| 1–505 | H-1 | L-6 | E-1 | 1–506 | H-124 | L-3 | E-1 |
| 1–507 | H-1 | L-7 | E-1 | 1–508 | H-125 | L-3 | E-1 |
| 1–509 | H-1 | L-8 | E-1 | 1–510 | H-126 | L-3 | E-1 |
| 1–511 | H-1 | L-9 | E-1 | 1–512 | H-127 | L-3 | E-1 |
| 1–513 | H-1 | L-10 | E-1 | 1–514 | H-128 | L-3 | E-1 |
| 1–515 | H-1 | L-11 | E-1 | 1–516 | H-129 | L-3 | E-1 |
| 1–517 | H-1 | L-12 | E-1 | 1–518 | H-130 | L-3 | E-1 |
| 1–519 | H-1 | L-13 | E-1 | 1–520 | H-131 | L-3 | E-1 |
| 1–521 | H-1 | L-14 | E-1 | 1–522 | H-132 | L-3 | E-1 |
| 1–523 | H-1 | L-15 | E-1 | 1–524 | H-133 | L-3 | E-1 |
| 1–525 | H-1 | L-16 | E-1 | 1–526 | H-134 | L-3 | E-1 |
| 1–527 | H-1 | L-17 | E-1 | 1–528 | H-135 | L-3 | E-1 |
| 1–529 | H-1 | L-18 | E-1 | 1–530 | H-136 | L-3 | E-1 |
| 1–531 | H-1 | L-19 | E-1 | 1–532 | H-137 | L-3 | E-1 |
| 1–533 | H-1 | L-20 | E-1 | 1–534 | H-138 | L-3 | E-1 |
| 1–535 | H-1 | L-21 | E-1 | 1–536 | H-139 | L-3 | E-1 |
| 1–537 | H-1 | L-22 | E-1 | 1–538 | H-139 | L-4 | E-7 |
| 1–539 | H-1 | L-0 | E-84 | 1–540 | H-139 | L-0 | E-85 |
| 1–541 | H-1 | L-0 | E-86 | 1–542 | H-1 | L-0 | E-91 |
| 1–543 | H-1 | L-0 | E-87 | 1–544 | H-1 | L-0 | E-92 |
| 1–545 | H-1 | L-0 | E-88 | 1–546 | H-1 | L-0 | E-93 |
| 1–547 | H-1 | L-0 | E-89 | 1–548 | H-1 | L-0 | E-94 |
| 1–549 | H-1 | L-0 | E-90 | 1–550 | H-1 | L-0 | E-95 |

wherein optionally, hydrogen ins Compound 1-1 to Compound 1-550 can be partially or fully substituted with deuterium.

9. The electroluminescent device according to claim 1, wherein in Formula 2, Y is, at each occurrence identically or differently, selected from C or CR_{Y};
wherein preferably, the second compound has a structure represented by one of Formulas 2-a to 2-d: 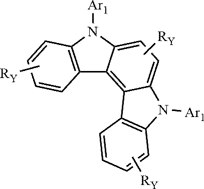 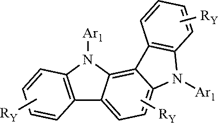 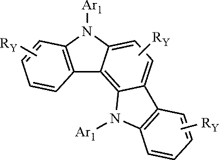 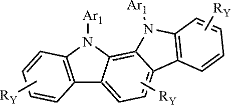
wherein R_{Y} represents, at each occurrence identically or differently, mono-substitution, multiple substitutions or non-substitution; R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents R_{Y} can be optionally joined to form a ring; and
more preferably, R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, a substituted or unsubstituted heterocyclic group having 3 to 20 ring atoms, substituted or unsubstituted aryl having 6 to 20 carbon atoms, substituted or unsubstituted heteroaryl having 3 to 20 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a cyano group, an isocyano group, a sulfanyl group, a sulfinyl group, a sulfonyl group, a phosphino group and combinations thereof.

10. The electroluminescent device according to claim 1, wherein Ar₁ is, at each occurrence identically or differently, selected from substituted or unsubstituted aryl having 6 to 25 carbon atoms, substituted or unsubstituted heteroaryl having 3 to 25 carbon atoms or a combination thereof;
preferably, Ar₁ is, at each occurrence identically or differently, selected from phenyl, fluorophenyl, naphthyl, biphenyl, benzothienyl, dibenzothienyl, benzofuranyl, dibenzofuranyl, dibenzoselenophenyl, carbazolyl, 9,9-dimethylfluorenyl, 9,9-spirobifluorenyl, cyanophenyl, phenanthryl, acridinyl, benzoacridinyl, adamantane spirofluorenyl or a combination thereof; and
more preferably, Ar₁ is, at each occurrence identically or differently, selected from the group consisting of the following structures: 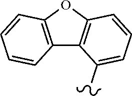 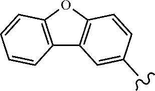 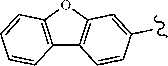 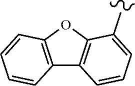 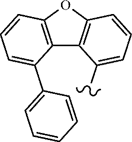 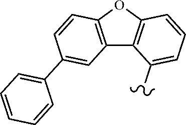 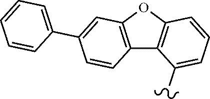 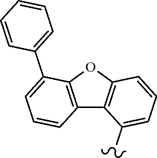 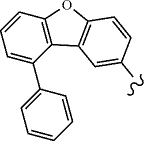 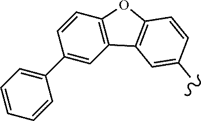 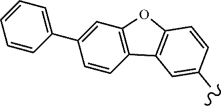 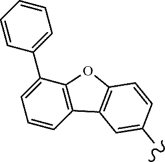 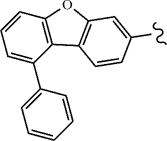 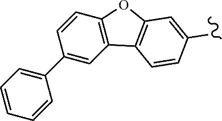 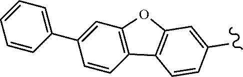 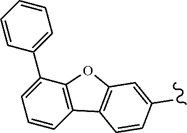 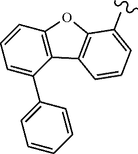 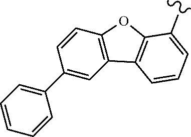 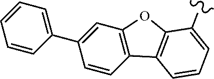 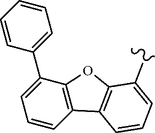 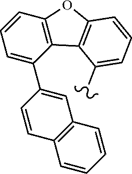 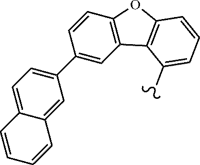 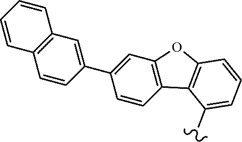 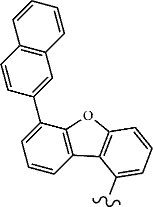 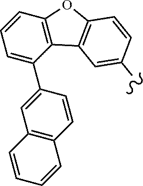 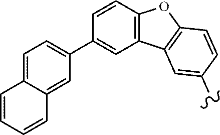 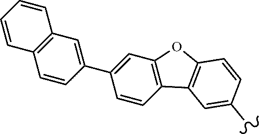 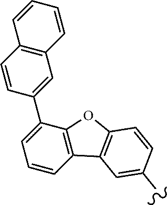 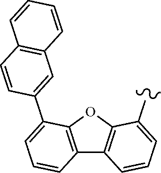 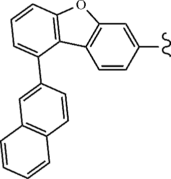 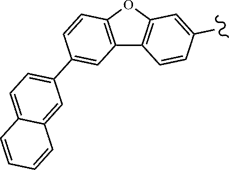 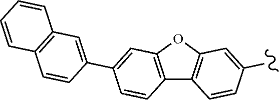 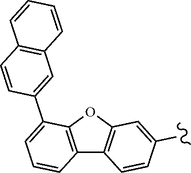 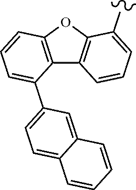 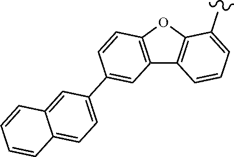 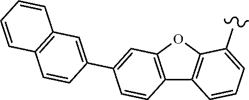 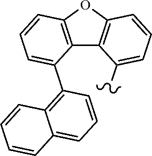 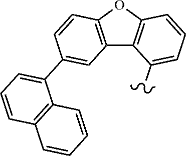 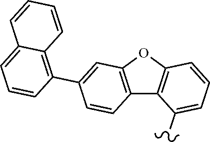 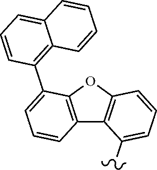 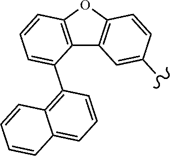 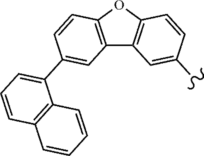 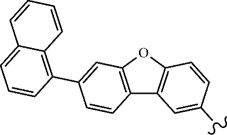 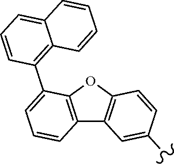 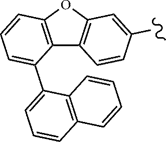 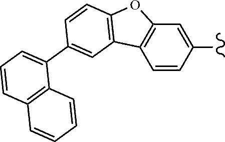 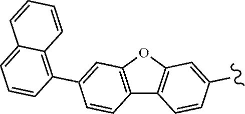 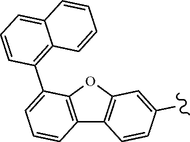 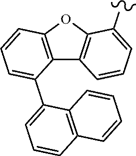 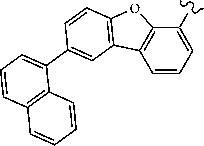 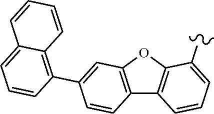 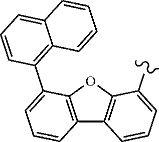 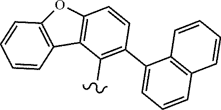 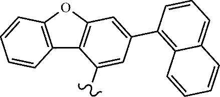 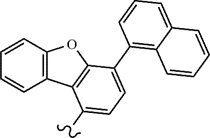 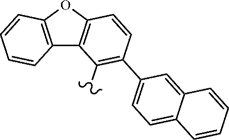 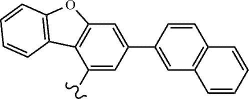 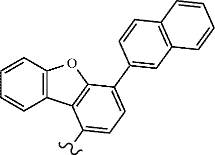 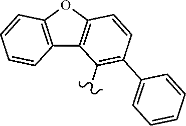 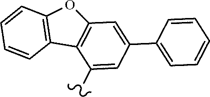 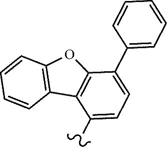 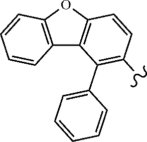 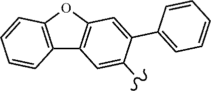 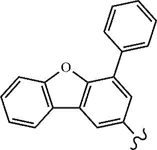 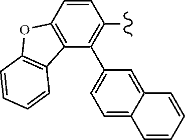 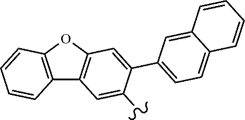 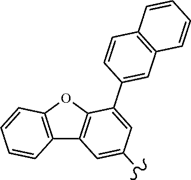 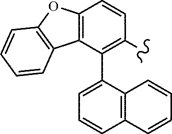 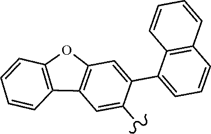 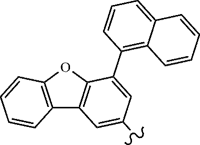 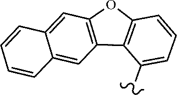 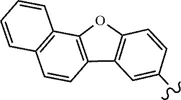 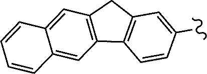 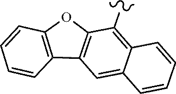 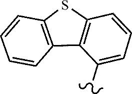 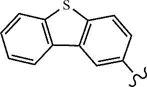 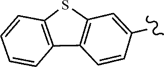 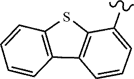 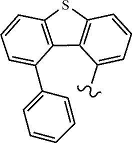 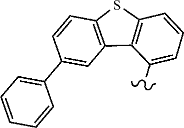 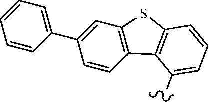 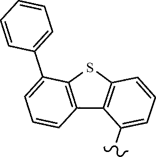 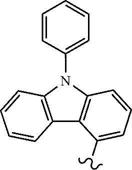 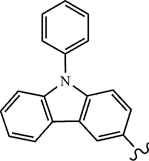 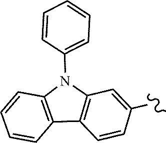 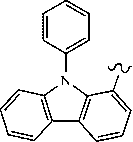 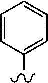 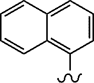 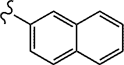 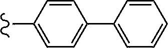 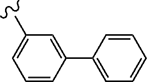 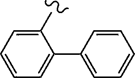 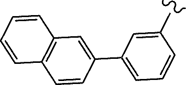 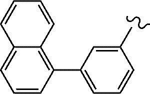 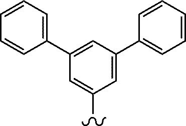 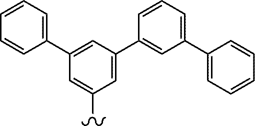 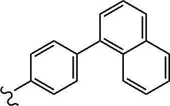 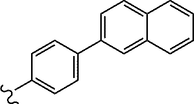 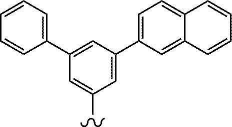 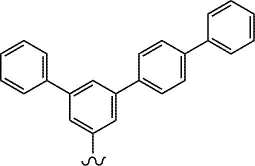 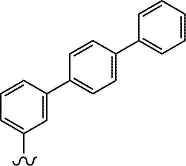 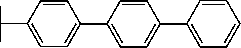 [SMILES: CC(C)c1ccc(-c2ccc(-c3ccccc3)cc2)cc1] 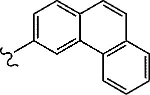 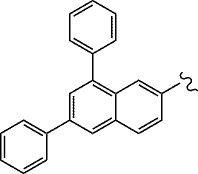 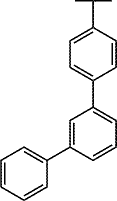 [SMILES: CC(C)c1ccc(-c2cccc(-c3ccccc3)c2)cc1] 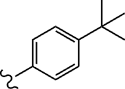 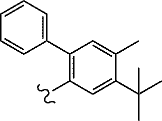 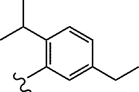 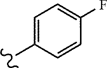 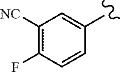 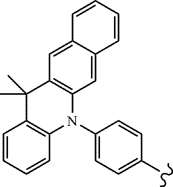 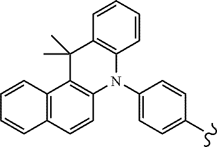 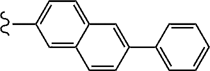 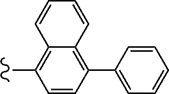 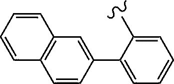 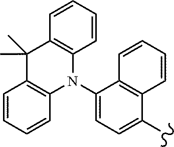 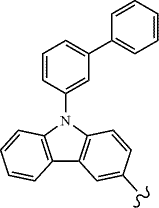 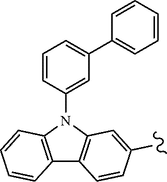 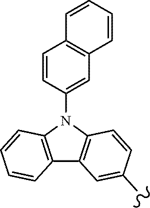 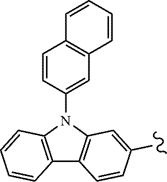 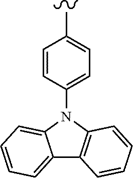 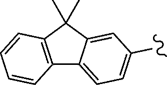 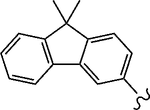 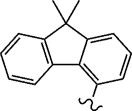 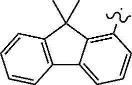 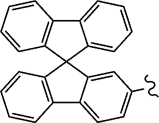 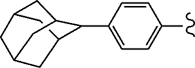 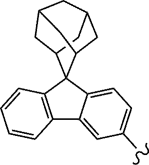 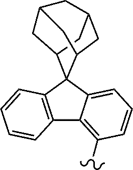 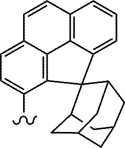 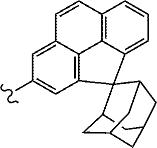 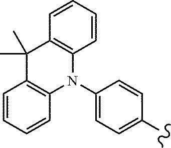
wherein optionally, hydrogens in the structures of Ar-1 to Ar-132 can be partially or fully substituted with deuterium.

11. The electroluminescent device according to claim 1, wherein the second compound is selected from the group consisting of the following structures: 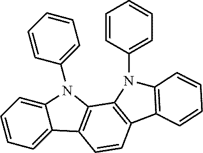 [SMILES: c1ccc(-n2c3ccccc3c3ccc4c5ccccc5n(-c5ccccc5)c4c32)cc1] 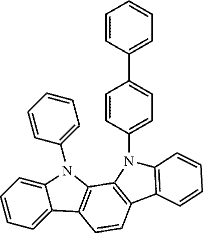 [SMILES: c1ccc(-c2ccc(-n3c4ccccc4c4ccc5c6ccccc6n(-c6ccccc6)c5c43)cc2)cc1] 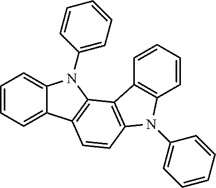 [SMILES: c1ccc(-n2c3ccccc3c3c2ccc2c4ccccc4n(-c4ccccc4)c23)cc1] 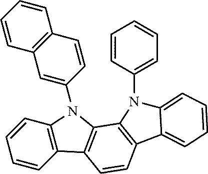 [SMILES: c1ccc(-n2c3ccccc3c3ccc4c5ccccc5n(-c5ccc6ccccc6c5)c4c32)cc1] 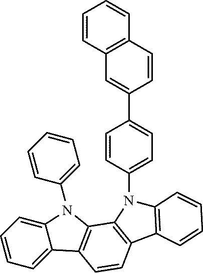 [SMILES: c1ccc(-n2c3ccccc3c3ccc4c5ccccc5n(-c5ccc(-c6ccc7ccccc7c6)cc5)c4c32)cc1] 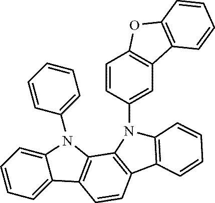 [SMILES: c1ccc(-n2c3ccccc3c3ccc4c5ccccc5n(-c5ccc6oc7ccccc7c6c5)c4c32)cc1] 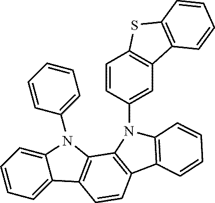 [SMILES: c1ccc(-n2c3ccccc3c3ccc4c5ccccc5n(-c5ccc6sc7ccccc7c6c5)c4c32)cc1] 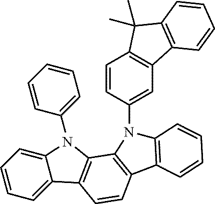 [SMILES: CC1(C)c2ccccc2-c2cc(-n3c4ccccc4c4ccc5c6ccccc6n(-c6ccccc6)c5c43)ccc21] 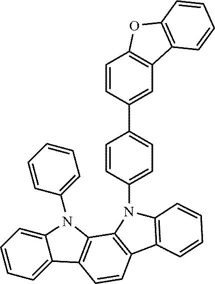 [SMILES: c1ccc(-n2c3ccccc3c3ccc4c5ccccc5n(-c5ccc(-c6ccc7oc8ccccc8c7c6)cc5)c4c32)cc1] 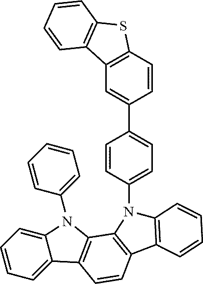 [SMILES: c1ccc(-n2c3ccccc3c3ccc4c5ccccc5n(-c5ccc(-c6ccc7sc8ccccc8c7c6)cc5)c4c32)cc1] 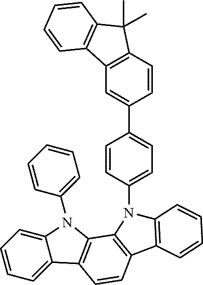 [SMILES: CC1(C)c2ccccc2-c2cc(-c3ccc(-n4c5ccccc5c5ccc6c7ccccc7n(-c7ccccc7)c6c54)cc3)ccc21] 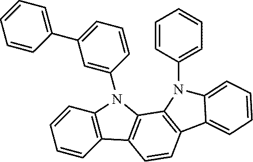 [SMILES: c1ccc(-c2cccc(-n3c4ccccc4c4ccc5c6ccccc6n(-c6ccccc6)c5c43)c2)cc1] 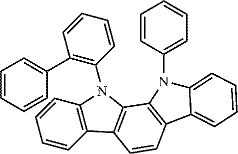 [SMILES: c1ccc(-c2ccccc2-n2c3ccccc3c3ccc4c5ccccc5n(-c5ccccc5)c4c32)cc1] 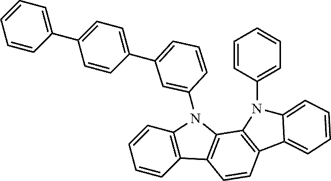 [SMILES: c1ccc(-c2ccc(-c3cccc(-n4c5ccccc5c5ccc6c7ccccc7n(-c7ccccc7)c6c54)c3)cc2)cc1] 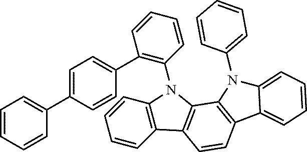 [SMILES: c1ccc(-c2ccc(-c3ccccc3-n3c4ccccc4c4ccc5c6ccccc6n(-c6ccccc6)c5c43)cc2)cc1] 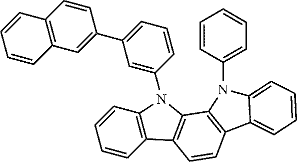 [SMILES: c1ccc(-n2c3ccccc3c3ccc4c5ccccc5n(-c5cccc(-c6ccc7ccccc7c6)c5)c4c32)cc1] 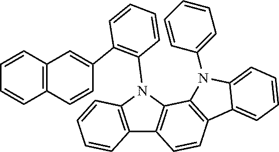 [SMILES: c1ccc(-n2c3ccccc3c3ccc4c5ccccc5n(-c5ccccc5-c5ccc6ccccc6c5)c4c32)cc1] 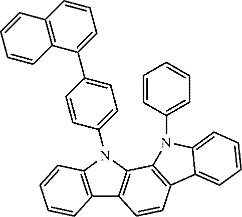 [SMILES: c1ccc(-n2c3ccccc3c3ccc4c5ccccc5n(-c5ccc(-c6cccc7ccccc67)cc5)c4c32)cc1] 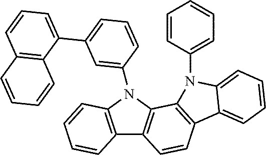 [SMILES: c1ccc(-n2c3ccccc3c3ccc4c5ccccc5n(-c5cccc(-c6cccc7ccccc67)c5)c4c32)cc1] 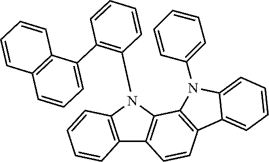 [SMILES: c1ccc(-n2c3ccccc3c3ccc4c5ccccc5n(-c5ccccc5-c5cccc6ccccc56)c4c32)cc1] 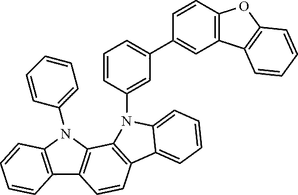 [SMILES: c1ccc(-n2c3ccccc3c3ccc4c5ccccc5n(-c5cccc(-c6ccc7oc8ccccc8c7c6)c5)c4c32)cc1] 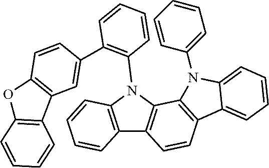 [SMILES: c1ccc(-n2c3ccccc3c3ccc4c5ccccc5n(-c5ccccc5-c5ccc6oc7ccccc7c6c5)c4c32)cc1] 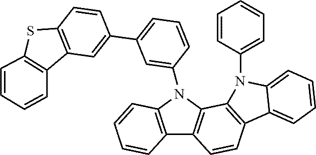 [SMILES: c1ccc(-n2c3ccccc3c3ccc4c5ccccc5n(-c5cccc(-c6ccc7sc8ccccc8c7c6)c5)c4c32)cc1] 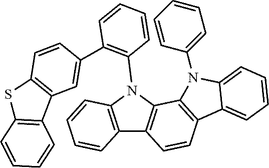 [SMILES: c1ccc(-n2c3ccccc3c3ccc4c5ccccc5n(-c5ccccc5-c5ccc6sc7ccccc7c6c5)c4c32)cc1] 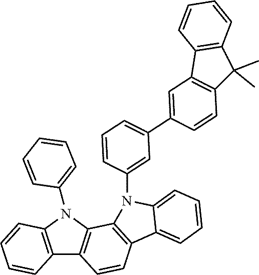 [SMILES: CC1(C)c2ccccc2-c2cc(-c3cccc(-n4c5ccccc5c5ccc6c7ccccc7n(-c7ccccc7)c6c54)c3)ccc21] 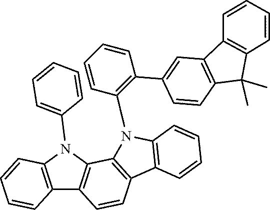 [SMILES: CC1(C)c2ccccc2-c2cc(-c3ccccc3-n3c4ccccc4c4ccc5c6ccccc6n(-c6ccccc6)c5c43)ccc21] 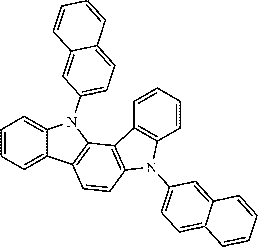 [SMILES: c1ccc2cc(-n3c4ccccc4c4c3ccc3c5ccccc5n(-c5ccc6ccccc6c5)c34)ccc2c1] 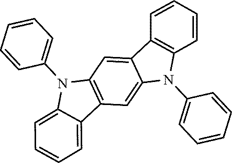 [SMILES: c1ccc(-n2c3ccccc3c3cc4c(cc32)c2ccccc2n4-c2ccccc2)cc1] 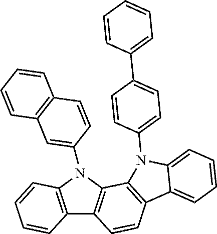 [SMILES: c1ccc(-c2ccc(-n3c4ccccc4c4ccc5c6ccccc6n(-c6ccc7ccccc7c6)c5c43)cc2)cc1] 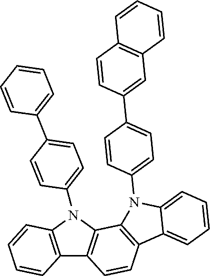 [SMILES: c1ccc(-c2ccc(-n3c4ccccc4c4ccc5c6ccccc6n(-c6ccc(-c7ccc8ccccc8c7)cc6)c5c43)cc2)cc1] 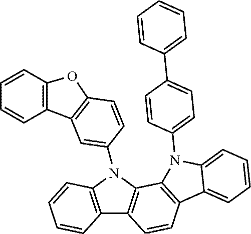 [SMILES: c1ccc(-c2ccc(-n3c4ccccc4c4ccc5c6ccccc6n(-c6ccc7oc8ccccc8c7c6)c5c43)cc2)cc1] 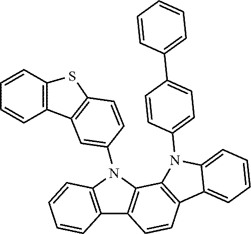 [SMILES: c1ccc(-c2ccc(-n3c4ccccc4c4ccc5c6ccccc6n(-c6ccc7sc8ccccc8c7c6)c5c43)cc2)cc1] 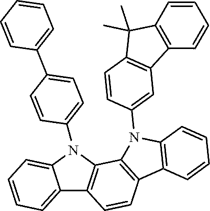 [SMILES: CC1(C)c2ccccc2-c2cc(-n3c4ccccc4c4ccc5c6ccccc6n(-c6ccc(-c7ccccc7)cc6)c5c43)ccc21] 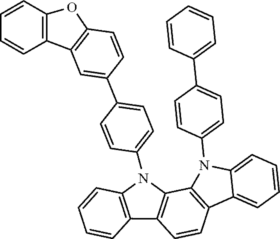 [SMILES: c1ccc(-c2ccc(-n3c4ccccc4c4ccc5c6ccccc6n(-c6ccc(-c7ccc8oc9ccccc9c8c7)cc6)c5c43)cc2)cc1] 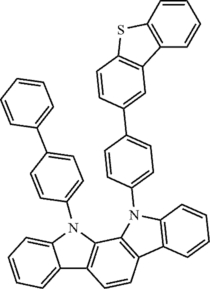 [SMILES: c1ccc(-c2ccc(-n3c4ccccc4c4ccc5c6ccccc6n(-c6ccc(-c7ccc8sc9ccccc9c8c7)cc6)c5c43)cc2)cc1] 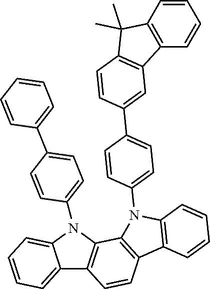 [SMILES: CC1(C)c2ccccc2-c2cc(-c3ccc(-n4c5ccccc5c5ccc6c7ccccc7n(-c7ccc(-c8ccccc8)cc7)c6c54)cc3)ccc21] 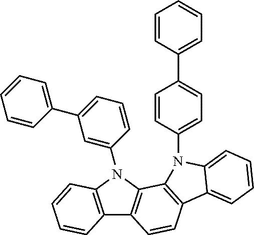 [SMILES: c1ccc(-c2ccc(-n3c4ccccc4c4ccc5c6ccccc6n(-c6cccc(-c7ccccc7)c6)c5c43)cc2)cc1] 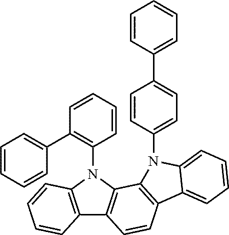 [SMILES: c1ccc(-c2ccc(-n3c4ccccc4c4ccc5c6ccccc6n(-c6ccccc6-c6ccccc6)c5c43)cc2)cc1] 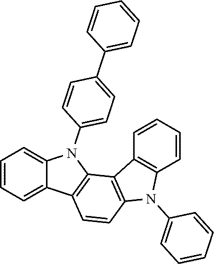 [SMILES: c1ccc(-c2ccc(-n3c4ccccc4c4ccc5c(c6ccccc6n5-c5ccccc5)c43)cc2)cc1] 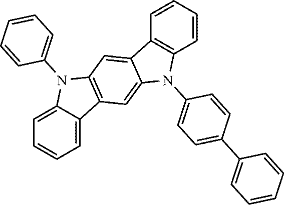 [SMILES: c1ccc(-c2ccc(-n3c4ccccc4c4cc5c(cc43)c3ccccc3n5-c3ccccc3)cc2)cc1] 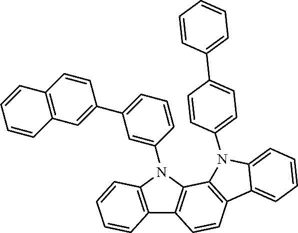 [SMILES: c1ccc(-c2ccc(-n3c4ccccc4c4ccc5c6ccccc6n(-c6cccc(-c7ccc8ccccc8c7)c6)c5c43)cc2)cc1] 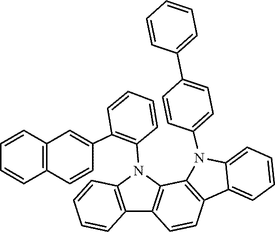 [SMILES: c1ccc(-c2ccc(-n3c4ccccc4c4ccc5c6ccccc6n(-c6ccccc6-c6ccc7ccccc7c6)c5c43)cc2)cc1] 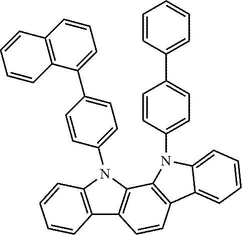 [SMILES: c1ccc(-c2ccc(-n3c4ccccc4c4ccc5c6ccccc6n(-c6ccc(-c7cccc8ccccc78)cc6)c5c43)cc2)cc1] 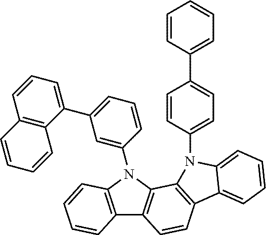 [SMILES: c1ccc(-c2ccc(-n3c4ccccc4c4ccc5c6ccccc6n(-c6cccc(-c7cccc8ccccc78)c6)c5c43)cc2)cc1] 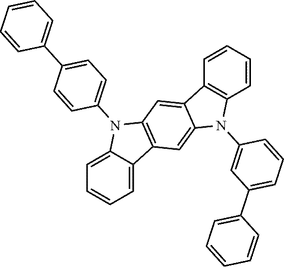 [SMILES: c1ccc(-c2ccc(-n3c4ccccc4c4cc5c(cc43)c3ccccc3n5-c3cccc(-c4ccccc4)c3)cc2)cc1] 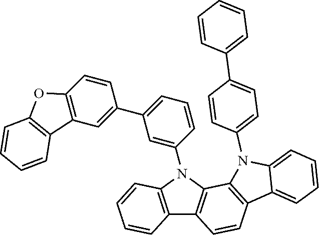 [SMILES: c1ccc(-c2ccc(-n3c4ccccc4c4ccc5c6ccccc6n(-c6cccc(-c7ccc8oc9ccccc9c8c7)c6)c5c43)cc2)cc1] 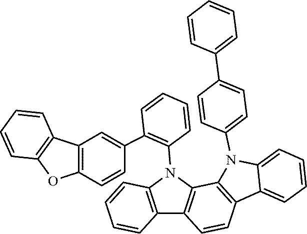 [SMILES: c1ccc(-c2ccc(-n3c4ccccc4c4ccc5c6ccccc6n(-c6ccccc6-c6ccc7oc8ccccc8c7c6)c5c43)cc2)cc1] 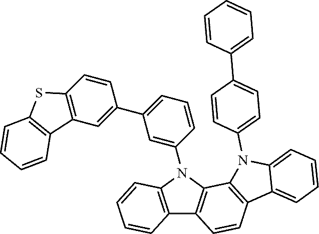 [SMILES: c1ccc(-c2ccc(-n3c4ccccc4c4ccc5c6ccccc6n(-c6cccc(-c7ccc8sc9ccccc9c8c7)c6)c5c43)cc2)cc1] 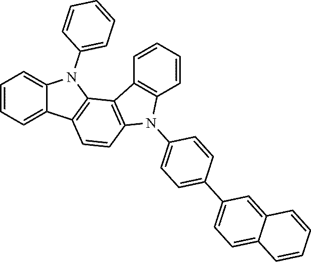 [SMILES: c1ccc(-n2c3ccccc3c3ccc4c(c5ccccc5n4-c4ccc(-c5ccc6ccccc6c5)cc4)c32)cc1] 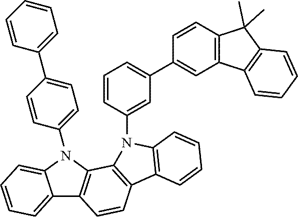 [SMILES: CC1(C)c2ccccc2-c2cc(-c3cccc(-n4c5ccccc5c5ccc6c7ccccc7n(-c7ccc(-c8ccccc8)cc7)c6c54)c3)ccc21] 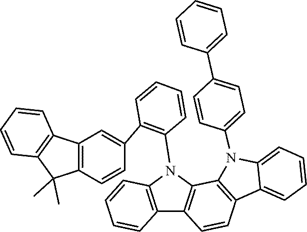 [SMILES: CC1(C)c2ccccc2-c2cc(-c3ccccc3-n3c4ccccc4c4ccc5c6ccccc6n(-c6ccc(-c7ccccc7)cc6)c5c43)ccc21] 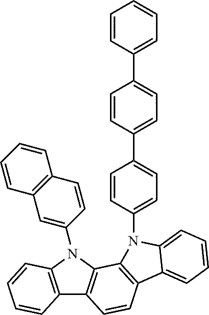 [SMILES: c1ccc(-c2ccc(-c3ccc(-n4c5ccccc5c5ccc6c7ccccc7n(-c7ccc8ccccc8c7)c6c54)cc3)cc2)cc1] 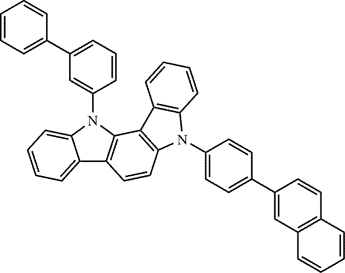 [SMILES: c1ccc(-c2cccc(-n3c4ccccc4c4ccc5c(c6ccccc6n5-c5ccc(-c6ccc7ccccc7c6)cc5)c43)c2)cc1] 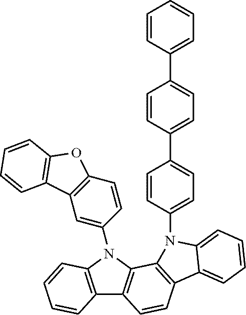 [SMILES: c1ccc(-c2ccc(-c3ccc(-n4c5ccccc5c5ccc6c7ccccc7n(-c7ccc8oc9ccccc9c8c7)c6c54)cc3)cc2)cc1] 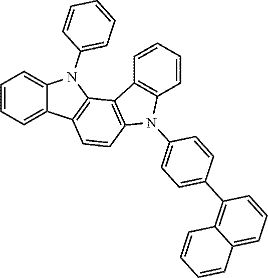 [SMILES: c1ccc(-n2c3ccccc3c3ccc4c(c5ccccc5n4-c4ccc(-c5cccc6ccccc56)cc4)c32)cc1] 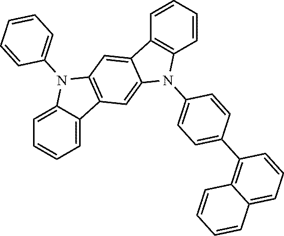 [SMILES: c1ccc(-n2c3ccccc3c3cc4c(cc32)c2ccccc2n4-c2ccc(-c3cccc4ccccc34)cc2)cc1] 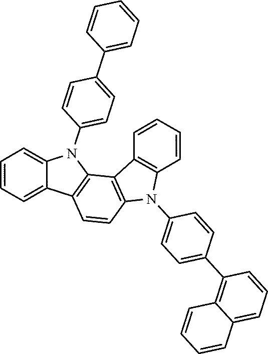 [SMILES: c1ccc(-c2ccc(-n3c4ccccc4c4ccc5c(c6ccccc6n5-c5ccc(-c6cccc7ccccc67)cc5)c43)cc2)cc1] 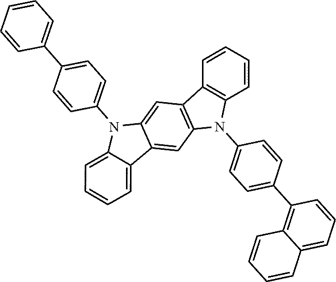 [SMILES: c1ccc(-c2ccc(-n3c4ccccc4c4cc5c(cc43)c3ccccc3n5-c3ccc(-c4cccc5ccccc45)cc3)cc2)cc1] 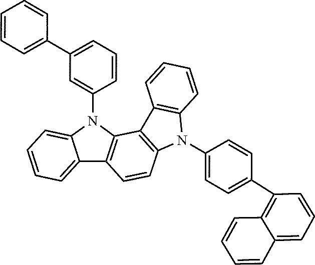 [SMILES: c1ccc(-c2cccc(-n3c4ccccc4c4ccc5c(c6ccccc6n5-c5ccc(-c6cccc7ccccc67)cc5)c43)c2)cc1] 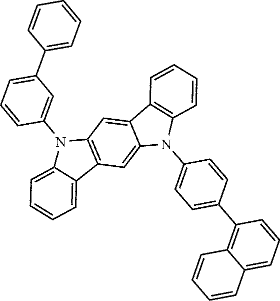 [SMILES: c1ccc(-c2cccc(-n3c4ccccc4c4cc5c(cc43)c3ccccc3n5-c3ccc(-c4cccc5ccccc45)cc3)c2)cc1] 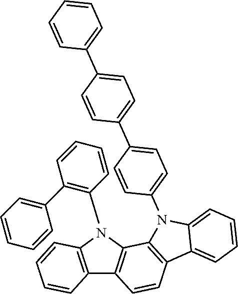 [SMILES: c1ccc(-c2ccc(-c3ccc(-n4c5ccccc5c5ccc6c7ccccc7n(-c7ccccc7-c7ccccc7)c6c54)cc3)cc2)cc1] 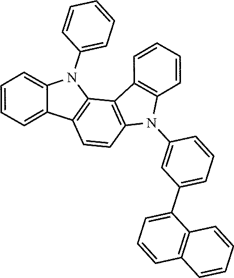 [SMILES: c1ccc(-n2c3ccccc3c3ccc4c(c5ccccc5n4-c4cccc(-c5cccc6ccccc56)c4)c32)cc1] 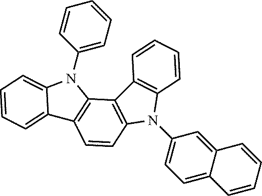 [SMILES: c1ccc(-n2c3ccccc3c3ccc4c(c5ccccc5n4-c4ccc5ccccc5c4)c32)cc1] 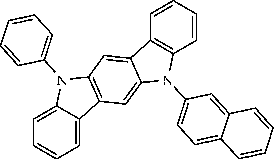 [SMILES: c1ccc(-n2c3ccccc3c3cc4c(cc32)c2ccccc2n4-c2ccc3ccccc3c2)cc1] 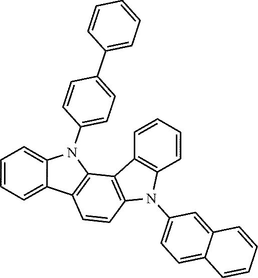 [SMILES: c1ccc(-c2ccc(-n3c4ccccc4c4ccc5c(c6ccccc6n5-c5ccc6ccccc6c5)c43)cc2)cc1] 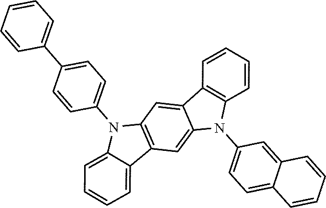 [SMILES: c1ccc(-c2ccc(-n3c4ccccc4c4cc5c(cc43)c3ccccc3n5-c3ccc4ccccc4c3)cc2)cc1] 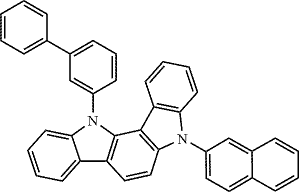 [SMILES: c1ccc(-c2cccc(-n3c4ccccc4c4ccc5c(c6ccccc6n5-c5ccc6ccccc6c5)c43)c2)cc1] 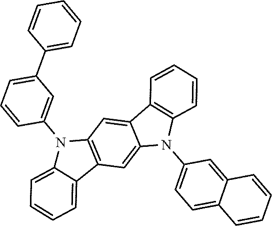 [SMILES: c1ccc(-c2cccc(-n3c4ccccc4c4cc5c(cc43)c3ccccc3n5-c3ccc4ccccc4c3)c2)cc1] 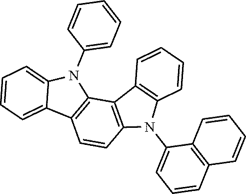 [SMILES: c1ccc(-n2c3ccccc3c3ccc4c(c5ccccc5n4-c4cccc5ccccc45)c32)cc1] 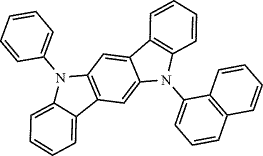 [SMILES: c1ccc(-n2c3ccccc3c3cc4c(cc32)c2ccccc2n4-c2cccc3ccccc23)cc1] 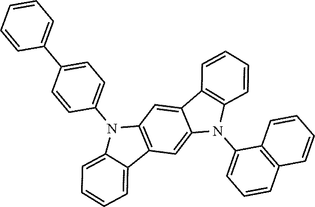 [SMILES: c1ccc(-c2ccc(-n3c4ccccc4c4cc5c(cc43)c3ccccc3n5-c3cccc4ccccc34)cc2)cc1] 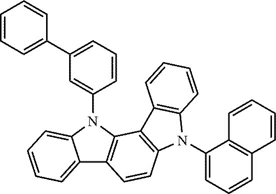 [SMILES: c1ccc(-c2cccc(-n3c4ccccc4c4ccc5c(c6ccccc6n5-c5cccc6ccccc56)c43)c2)cc1] 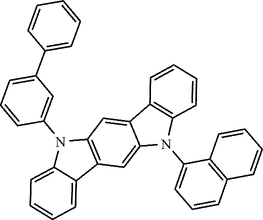 [SMILES: c1ccc(-c2cccc(-n3c4ccccc4c4cc5c(cc43)c3ccccc3n5-c3cccc4ccccc34)c2)cc1] 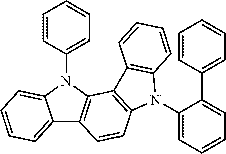 [SMILES: c1ccc(-c2ccccc2-n2c3ccccc3c3c2ccc2c4ccccc4n(-c4ccccc4)c23)cc1] 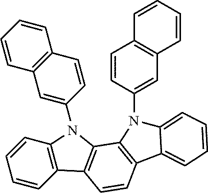 [SMILES: c1ccc2cc(-n3c4ccccc4c4ccc5c6ccccc6n(-c6ccc7ccccc7c6)c5c43)ccc2c1] 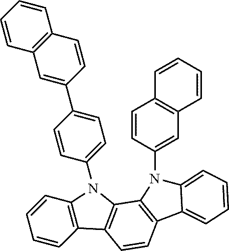 [SMILES: c1ccc2cc(-c3ccc(-n4c5ccccc5c5ccc6c7ccccc7n(-c7ccc8ccccc8c7)c6c54)cc3)ccc2c1] 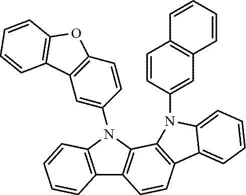 [SMILES: c1ccc2cc(-n3c4ccccc4c4ccc5c6ccccc6n(-c6ccc7oc8ccccc8c7c6)c5c43)ccc2c1] 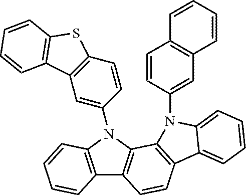 [SMILES: c1ccc2cc(-n3c4ccccc4c4ccc5c6ccccc6n(-c6ccc7sc8ccccc8c7c6)c5c43)ccc2c1] 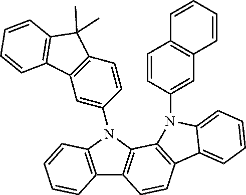 [SMILES: CC1(C)c2ccccc2-c2cc(-n3c4ccccc4c4ccc5c6ccccc6n(-c6ccc7ccccc7c6)c5c43)ccc21] 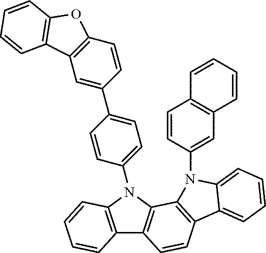 [SMILES: c1ccc2cc(-n3c4ccccc4c4ccc5c6ccccc6n(-c6ccc(-c7ccc8oc9ccccc9c8c7)cc6)c5c43)ccc2c1] 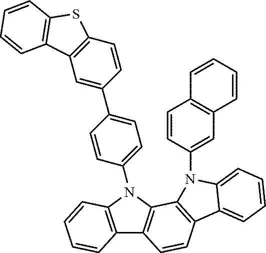 [SMILES: c1ccc2cc(-n3c4ccccc4c4ccc5c6ccccc6n(-c6ccc(-c7ccc8sc9ccccc9c8c7)cc6)c5c43)ccc2c1] 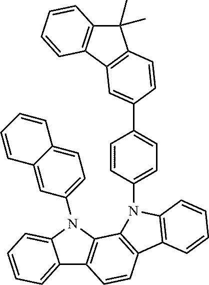 [SMILES: CC1(C)c2ccccc2-c2cc(-c3ccc(-n4c5ccccc5c5ccc6c7ccccc7n(-c7ccc8ccccc8c7)c6c54)cc3)ccc21] 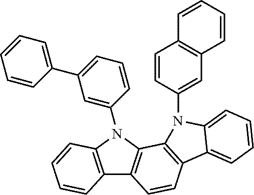 [SMILES: c1ccc(-c2cccc(-n3c4ccccc4c4ccc5c6ccccc6n(-c6ccc7ccccc7c6)c5c43)c2)cc1] 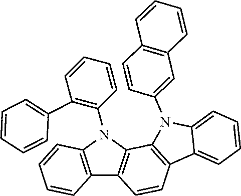 [SMILES: c1ccc(-c2ccccc2-n2c3ccccc3c3ccc4c5ccccc5n(-c5ccc6ccccc6c5)c4c32)cc1] 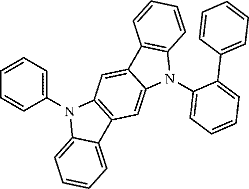 [SMILES: c1ccc(-c2ccccc2-n2c3ccccc3c3cc4c(cc32)c2ccccc2n4-c2ccccc2)cc1] 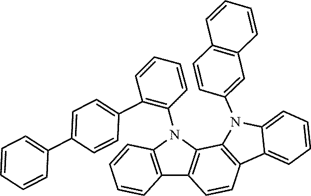 [SMILES: c1ccc(-c2ccc(-c3ccccc3-n3c4ccccc4c4ccc5c6ccccc6n(-c6ccc7ccccc7c6)c5c43)cc2)cc1] 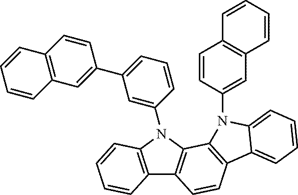 [SMILES: c1cc(-c2ccc3ccccc3c2)cc(-n2c3ccccc3c3ccc4c5ccccc5n(-c5ccc6ccccc6c5)c4c32)c1] 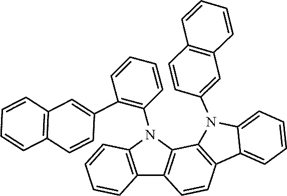 [SMILES: c1ccc(-n2c3ccccc3c3ccc4c5ccccc5n(-c5ccc6ccccc6c5)c4c32)c(-c2ccc3ccccc3c2)c1] 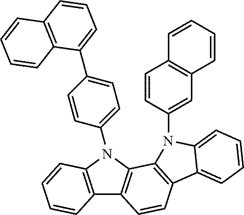 [SMILES: c1ccc2cc(-n3c4ccccc4c4ccc5c6ccccc6n(-c6ccc(-c7cccc8ccccc78)cc6)c5c43)ccc2c1] 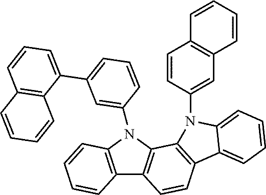 [SMILES: c1cc(-c2cccc3ccccc23)cc(-n2c3ccccc3c3ccc4c5ccccc5n(-c5ccc6ccccc6c5)c4c32)c1] 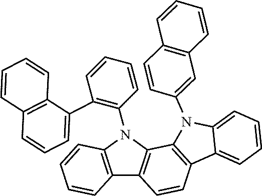 [SMILES: c1ccc(-n2c3ccccc3c3ccc4c5ccccc5n(-c5ccc6ccccc6c5)c4c32)c(-c2cccc3ccccc23)c1] 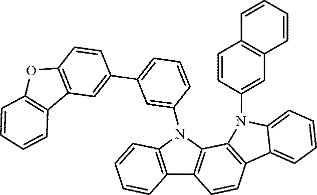 [SMILES: c1cc(-c2ccc3oc4ccccc4c3c2)cc(-n2c3ccccc3c3ccc4c5ccccc5n(-c5ccc6ccccc6c5)c4c32)c1] 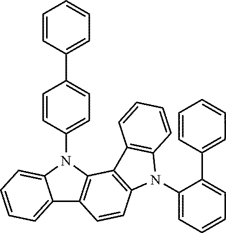 [SMILES: c1ccc(-c2ccc(-n3c4ccccc4c4ccc5c(c6ccccc6n5-c5ccccc5-c5ccccc5)c43)cc2)cc1] 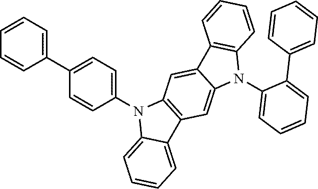 [SMILES: c1ccc(-c2ccc(-n3c4ccccc4c4cc5c(cc43)c3ccccc3n5-c3ccccc3-c3ccccc3)cc2)cc1] 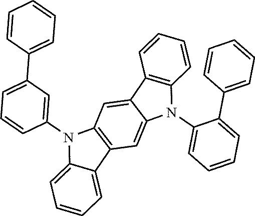 [SMILES: c1ccc(-c2cccc(-n3c4ccccc4c4cc5c(cc43)c3ccccc3n5-c3ccccc3-c3ccccc3)c2)cc1] 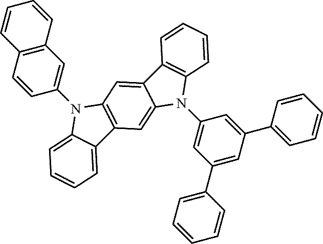 [SMILES: c1ccc(-c2cc(-c3ccccc3)cc(-n3c4ccccc4c4cc5c(cc43)c3ccccc3n5-c3ccc4ccccc4c3)c2)cc1] 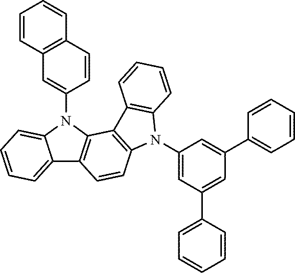 [SMILES: c1ccc(-c2cc(-c3ccccc3)cc(-n3c4ccccc4c4c3ccc3c5ccccc5n(-c5ccc6ccccc6c5)c34)c2)cc1] 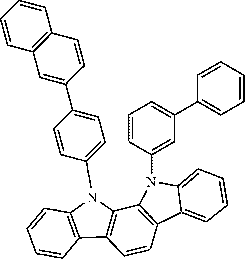 [SMILES: c1ccc(-c2cccc(-n3c4ccccc4c4ccc5c6ccccc6n(-c6ccc(-c7ccc8ccccc8c7)cc6)c5c43)c2)cc1] 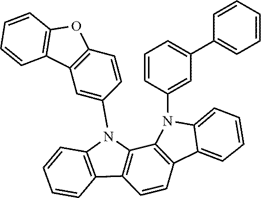 [SMILES: c1ccc(-c2cccc(-n3c4ccccc4c4ccc5c6ccccc6n(-c6ccc7oc8ccccc8c7c6)c5c43)c2)cc1] 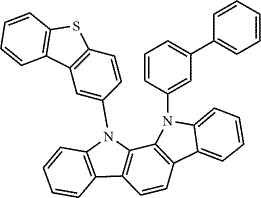 [SMILES: c1ccc(-c2cccc(-n3c4ccccc4c4ccc5c6ccccc6n(-c6ccc7sc8ccccc8c7c6)c5c43)c2)cc1] 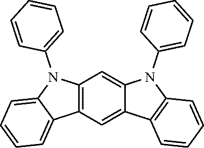 [SMILES: c1ccc(-n2c3ccccc3c3cc4c5ccccc5n(-c5ccccc5)c4cc32)cc1] 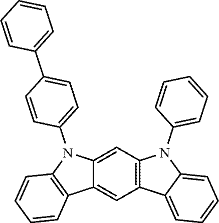 [SMILES: c1ccc(-c2ccc(-n3c4ccccc4c4cc5c6ccccc6n(-c6ccccc6)c5cc43)cc2)cc1] 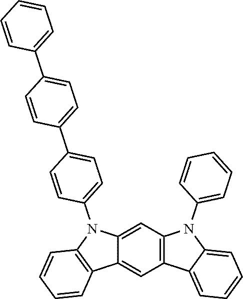 [SMILES: c1ccc(-c2ccc(-c3ccc(-n4c5ccccc5c5cc6c7ccccc7n(-c7ccccc7)c6cc54)cc3)cc2)cc1] 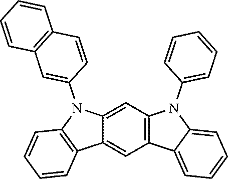 [SMILES: c1ccc(-n2c3ccccc3c3cc4c5ccccc5n(-c5ccc6ccccc6c5)c4cc32)cc1] 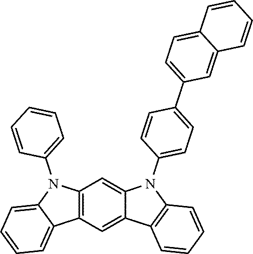 [SMILES: c1ccc(-n2c3ccccc3c3cc4c5ccccc5n(-c5ccc(-c6ccc7ccccc7c6)cc5)c4cc32)cc1] 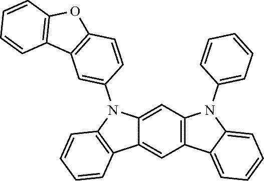 [SMILES: c1ccc(-n2c3ccccc3c3cc4c5ccccc5n(-c5ccc6oc7ccccc7c6c5)c4cc32)cc1] 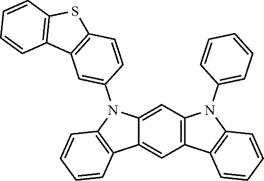 [SMILES: c1ccc(-n2c3ccccc3c3cc4c5ccccc5n(-c5ccc6sc7ccccc7c6c5)c4cc32)cc1] 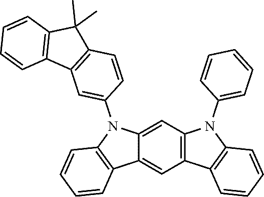 [SMILES: CC1(C)c2ccccc2-c2cc(-n3c4ccccc4c4cc5c6ccccc6n(-c6ccccc6)c5cc43)ccc21] 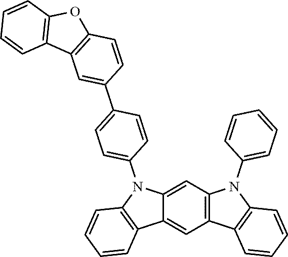 [SMILES: c1ccc(-n2c3ccccc3c3cc4c5ccccc5n(-c5ccc(-c6ccc7oc8ccccc8c7c6)cc5)c4cc32)cc1] 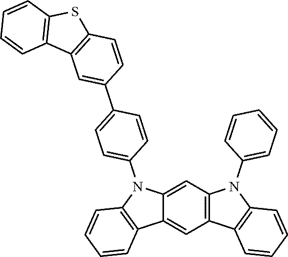 [SMILES: c1ccc(-n2c3ccccc3c3cc4c5ccccc5n(-c5ccc(-c6ccc7sc8ccccc8c7c6)cc5)c4cc32)cc1] 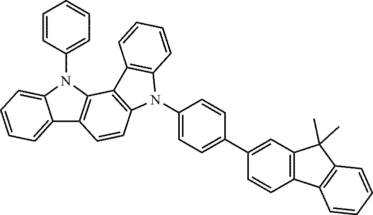 [SMILES: CC1(C)c2ccccc2-c2ccc(-c3ccc(-n4c5ccccc5c5c4ccc4c6ccccc6n(-c6ccccc6)c45)cc3)cc21] 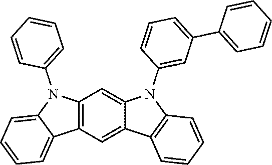 [SMILES: c1ccc(-c2cccc(-n3c4ccccc4c4cc5c6ccccc6n(-c6ccccc6)c5cc43)c2)cc1] 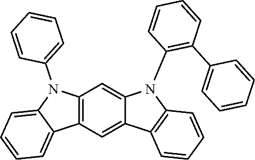 [SMILES: c1ccc(-c2ccccc2-n2c3ccccc3c3cc4c5ccccc5n(-c5ccccc5)c4cc32)cc1] 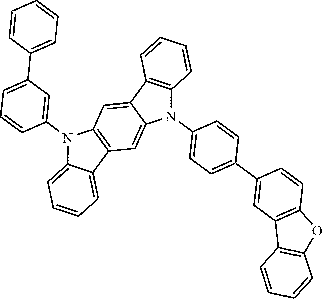 [SMILES: c1ccc(-c2cccc(-n3c4ccccc4c4cc5c(cc43)c3ccccc3n5-c3ccc(-c4ccc5oc6ccccc6c5c4)cc3)c2)cc1] 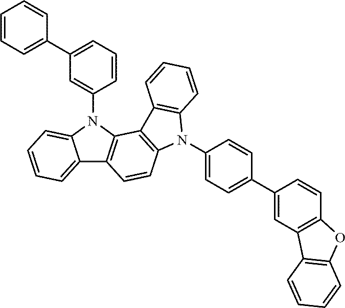 [SMILES: c1ccc(-c2cccc(-n3c4ccccc4c4ccc5c(c6ccccc6n5-c5ccc(-c6ccc7oc8ccccc8c7c6)cc5)c43)c2)cc1] 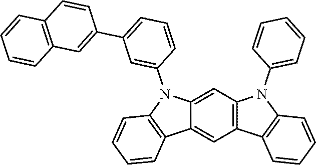 [SMILES: c1ccc(-n2c3ccccc3c3cc4c5ccccc5n(-c5cccc(-c6ccc7ccccc7c6)c5)c4cc32)cc1] 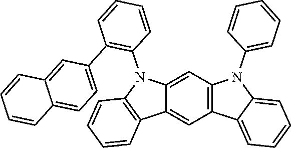 [SMILES: c1ccc(-n2c3ccccc3c3cc4c5ccccc5n(-c5ccccc5-c5ccc6ccccc6c5)c4cc32)cc1] 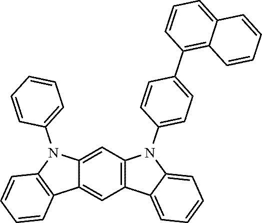 [SMILES: c1ccc(-n2c3ccccc3c3cc4c5ccccc5n(-c5ccc(-c6cccc7ccccc67)cc5)c4cc32)cc1] 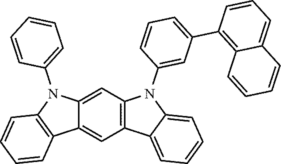 [SMILES: c1ccc(-n2c3ccccc3c3cc4c5ccccc5n(-c5cccc(-c6cccc7ccccc67)c5)c4cc32)cc1] 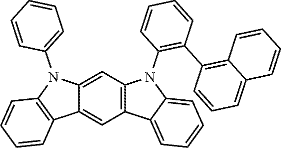 [SMILES: c1ccc(-n2c3ccccc3c3cc4c5ccccc5n(-c5ccccc5-c5cccc6ccccc56)c4cc32)cc1] 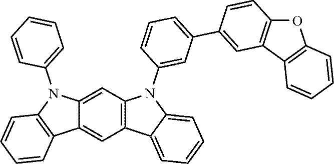 [SMILES: c1ccc(-n2c3ccccc3c3cc4c5ccccc5n(-c5cccc(-c6ccc7oc8ccccc8c7c6)c5)c4cc32)cc1] 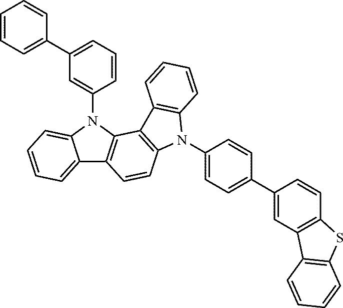 [SMILES: c1ccc(-c2cccc(-n3c4ccccc4c4ccc5c(c6ccccc6n5-c5ccc(-c6ccc7sc8ccccc8c7c6)cc5)c43)c2)cc1] 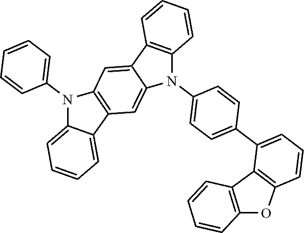 [SMILES: c1ccc(-n2c3ccccc3c3cc4c(cc32)c2ccccc2n4-c2ccc(-c3cccc4oc5ccccc5c34)cc2)cc1] 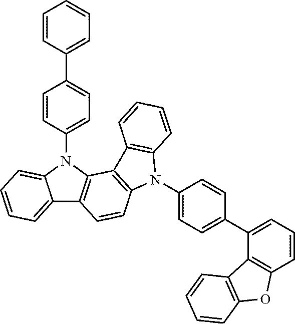 [SMILES: c1ccc(-c2ccc(-n3c4ccccc4c4ccc5c(c6ccccc6n5-c5ccc(-c6cccc7oc8ccccc8c67)cc5)c43)cc2)cc1] 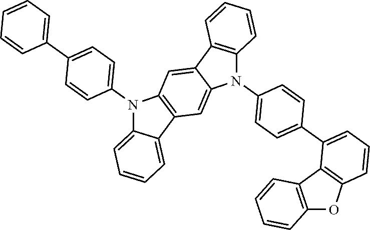 [SMILES: c1ccc(-c2ccc(-n3c4ccccc4c4cc5c(cc43)c3ccccc3n5-c3ccc(-c4cccc5oc6ccccc6c45)cc3)cc2)cc1] 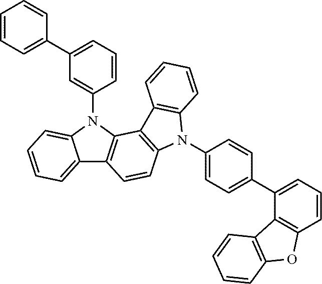 [SMILES: c1ccc(-c2cccc(-n3c4ccccc4c4ccc5c(c6ccccc6n5-c5ccc(-c6cccc7oc8ccccc8c67)cc5)c43)c2)cc1] 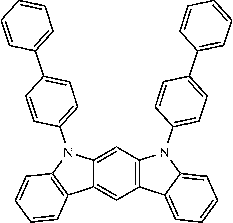 [SMILES: c1ccc(-c2ccc(-n3c4ccccc4c4cc5c6ccccc6n(-c6ccc(-c7ccccc7)cc6)c5cc43)cc2)cc1] 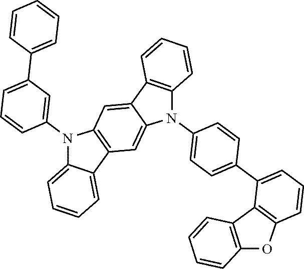 [SMILES: c1ccc(-c2cccc(-n3c4ccccc4c4cc5c(cc43)c3ccccc3n5-c3ccc(-c4cccc5oc6ccccc6c45)cc3)c2)cc1] 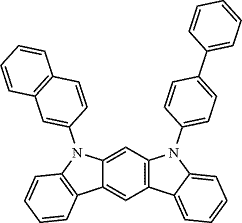 [SMILES: c1ccc(-c2ccc(-n3c4ccccc4c4cc5c6ccccc6n(-c6ccc7ccccc7c6)c5cc43)cc2)cc1] 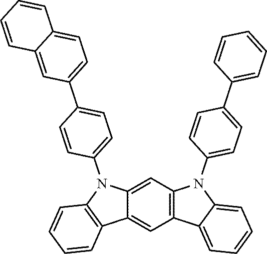 [SMILES: c1ccc(-c2ccc(-n3c4ccccc4c4cc5c6ccccc6n(-c6ccc(-c7ccc8ccccc8c7)cc6)c5cc43)cc2)cc1] 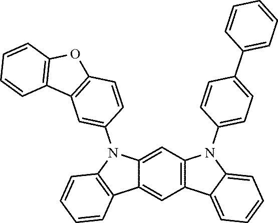 [SMILES: c1ccc(-c2ccc(-n3c4ccccc4c4cc5c6ccccc6n(-c6ccc7oc8ccccc8c7c6)c5cc43)cc2)cc1] 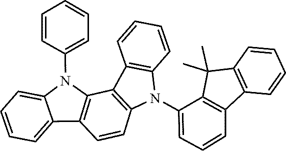 [SMILES: CC1(C)c2ccccc2-c2cccc(-n3c4ccccc4c4c3ccc3c5ccccc5n(-c5ccccc5)c34)c21] 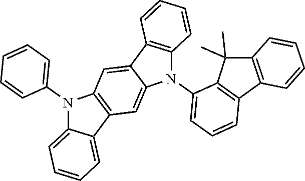 [SMILES: CC1(C)c2ccccc2-c2cccc(-n3c4ccccc4c4cc5c(cc43)c3ccccc3n5-c3ccccc3)c21] 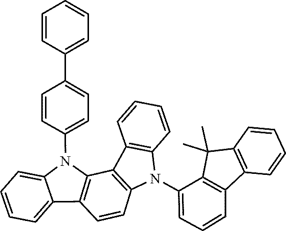 [SMILES: CC1(C)c2ccccc2-c2cccc(-n3c4ccccc4c4c3ccc3c5ccccc5n(-c5ccc(-c6ccccc6)cc5)c34)c21] 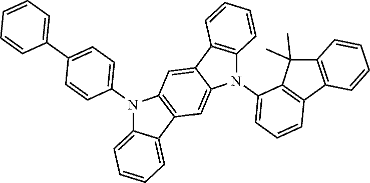 [SMILES: CC1(C)c2ccccc2-c2cccc(-n3c4ccccc4c4cc5c(cc43)c3ccccc3n5-c3ccc(-c4ccccc4)cc3)c21] 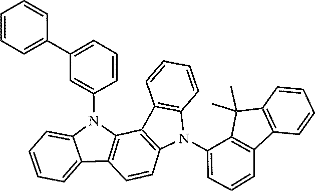 [SMILES: CC1(C)c2ccccc2-c2cccc(-n3c4ccccc4c4c3ccc3c5ccccc5n(-c5cccc(-c6ccccc6)c5)c34)c21] 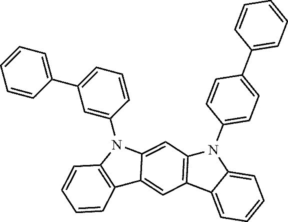 [SMILES: c1ccc(-c2ccc(-n3c4ccccc4c4cc5c6ccccc6n(-c6cccc(-c7ccccc7)c6)c5cc43)cc2)cc1] 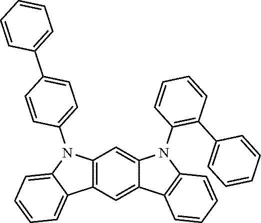 [SMILES: c1ccc(-c2ccc(-n3c4ccccc4c4cc5c6ccccc6n(-c6ccccc6-c6ccccc6)c5cc43)cc2)cc1] 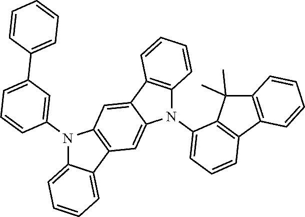 [SMILES: CC1(C)c2ccccc2-c2cccc(-n3c4ccccc4c4cc5c(cc43)c3ccccc3n5-c3cccc(-c4ccccc4)c3)c21] 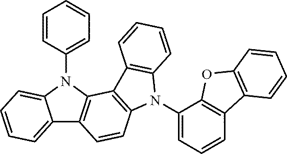 [SMILES: c1ccc(-n2c3ccccc3c3ccc4c(c5ccccc5n4-c4cccc5c4oc4ccccc45)c32)cc1] 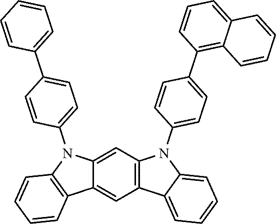 [SMILES: c1ccc(-c2ccc(-n3c4ccccc4c4cc5c6ccccc6n(-c6ccc(-c7cccc8ccccc78)cc6)c5cc43)cc2)cc1] 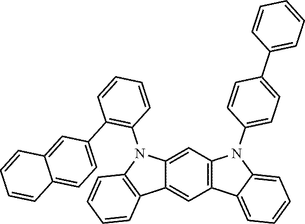 [SMILES: c1ccc(-c2ccc(-n3c4ccccc4c4cc5c6ccccc6n(-c6ccccc6-c6ccc7ccccc7c6)c5cc43)cc2)cc1] 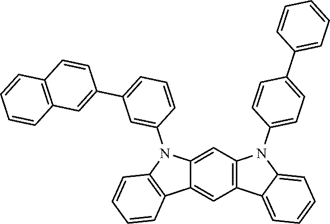 [SMILES: c1ccc(-c2ccc(-n3c4ccccc4c4cc5c6ccccc6n(-c6cccc(-c7ccc8ccccc8c7)c6)c5cc43)cc2)cc1] 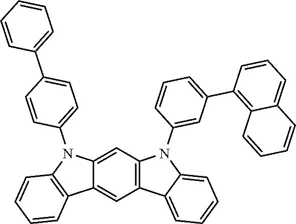 [SMILES: c1ccc(-c2ccc(-n3c4ccccc4c4cc5c6ccccc6n(-c6cccc(-c7cccc8ccccc78)c6)c5cc43)cc2)cc1] 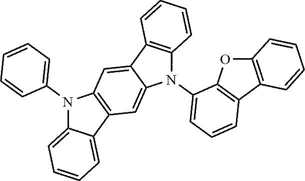 [SMILES: c1ccc(-n2c3ccccc3c3cc4c(cc32)c2ccccc2n4-c2cccc3c2oc2ccccc23)cc1] 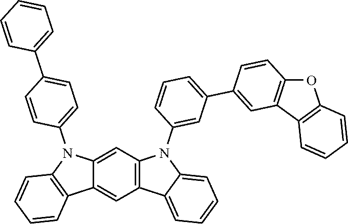 [SMILES: c1ccc(-c2ccc(-n3c4ccccc4c4cc5c6ccccc6n(-c6cccc(-c7ccc8oc9ccccc9c8c7)c6)c5cc43)cc2)cc1] 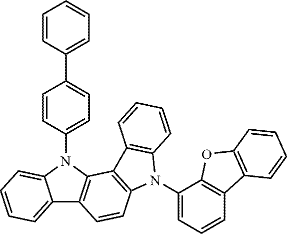 [SMILES: c1ccc(-c2ccc(-n3c4ccccc4c4ccc5c(c6ccccc6n5-c5cccc6c5oc5ccccc56)c43)cc2)cc1] 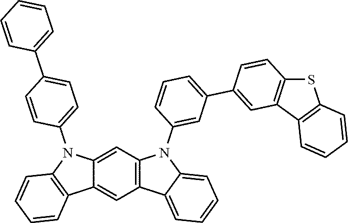 [SMILES: c1ccc(-c2ccc(-n3c4ccccc4c4cc5c6ccccc6n(-c6cccc(-c7ccc8sc9ccccc9c8c7)c6)c5cc43)cc2)cc1] 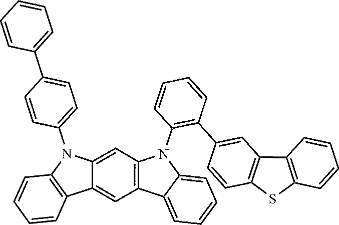 [SMILES: c1ccc(-c2ccc(-n3c4ccccc4c4cc5c6ccccc6n(-c6ccccc6-c6ccc7sc8ccccc8c7c6)c5cc43)cc2)cc1] 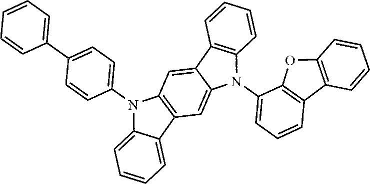 [SMILES: c1ccc(-c2ccc(-n3c4ccccc4c4cc5c(cc43)c3ccccc3n5-c3cccc4c3oc3ccccc34)cc2)cc1] 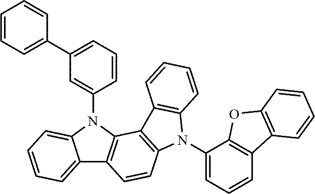 [SMILES: c1ccc(-c2cccc(-n3c4ccccc4c4ccc5c(c6ccccc6n5-c5cccc6c5oc5ccccc56)c43)c2)cc1] 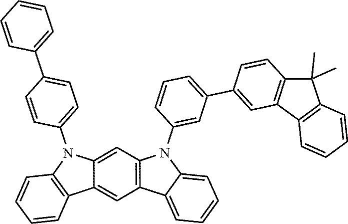 [SMILES: CC1(C)c2ccccc2-c2cc(-c3cccc(-n4c5ccccc5c5cc6c7ccccc7n(-c7ccc(-c8ccccc8)cc7)c6cc54)c3)ccc21] 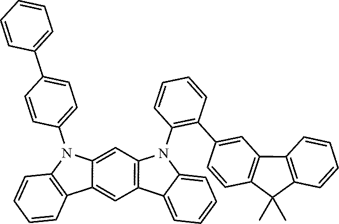 [SMILES: CC1(C)c2ccccc2-c2cc(-c3ccccc3-n3c4ccccc4c4cc5c6ccccc6n(-c6ccc(-c7ccccc7)cc6)c5cc43)ccc21] 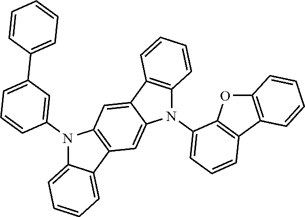 [SMILES: c1ccc(-c2cccc(-n3c4ccccc4c4cc5c(cc43)c3ccccc3n5-c3cccc4c3oc3ccccc34)c2)cc1] 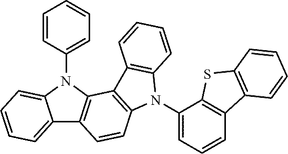 [SMILES: c1ccc(-n2c3ccccc3c3ccc4c(c5ccccc5n4-c4cccc5c4sc4ccccc45)c32)cc1] 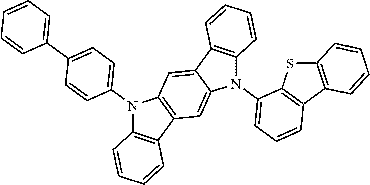 [SMILES: c1ccc(-c2ccc(-n3c4ccccc4c4cc5c(cc43)c3ccccc3n5-c3cccc4c3sc3ccccc34)cc2)cc1] 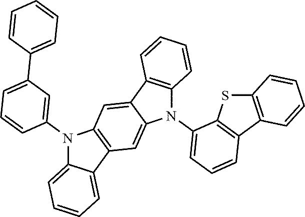 [SMILES: c1ccc(-c2cccc(-n3c4ccccc4c4cc5c(cc43)c3ccccc3n5-c3cccc4c3sc3ccccc34)c2)cc1] 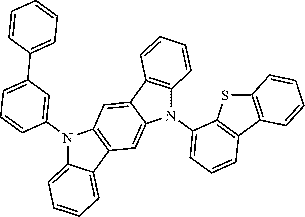 [SMILES: c1ccc(-c2cccc(-n3c4ccccc4c4cc5c(cc43)c3ccccc3n5-c3cccc4c3sc3ccccc34)c2)cc1] 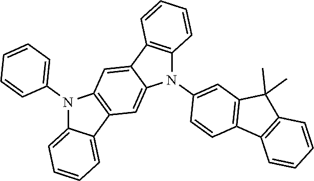 [SMILES: CC1(C)c2ccccc2-c2ccc(-n3c4ccccc4c4cc5c(cc43)c3ccccc3n5-c3ccccc3)cc21] 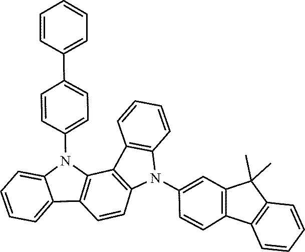 [SMILES: CC1(C)c2ccccc2-c2ccc(-n3c4ccccc4c4c3ccc3c5ccccc5n(-c5ccc(-c6ccccc6)cc5)c34)cc21] 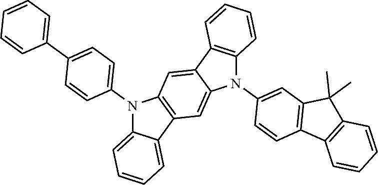 [SMILES: CC1(C)c2ccccc2-c2ccc(-n3c4ccccc4c4cc5c(cc43)c3ccccc3n5-c3ccc(-c4ccccc4)cc3)cc21] 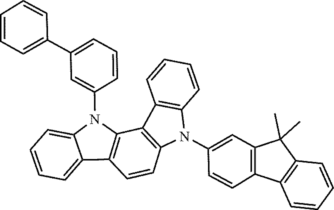 [SMILES: CC1(C)c2ccccc2-c2ccc(-n3c4ccccc4c4c3ccc3c5ccccc5n(-c5cccc(-c6ccccc6)c5)c34)cc21] 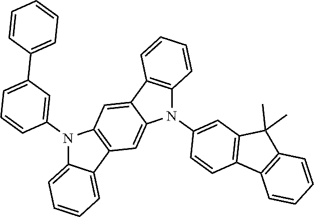 [SMILES: CC1(C)c2ccccc2-c2ccc(-n3c4ccccc4c4cc5c(cc43)c3ccccc3n5-c3cccc(-c4ccccc4)c3)cc21] 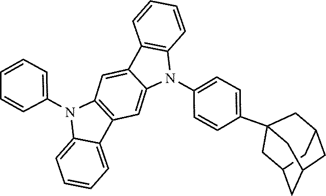 [SMILES: c1ccc(-n2c3ccccc3c3cc4c(cc32)c2ccccc2n4-c2ccc(C34CC5CC(CC(C5)C3)C4)cc2)cc1] 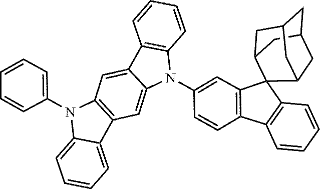 [SMILES: c1ccc(-n2c3ccccc3c3cc4c(cc32)c2ccccc2n4-c2ccc3c(c2)C2(c4ccccc4-3)C3CC4CC(C3)CC2C4)cc1] 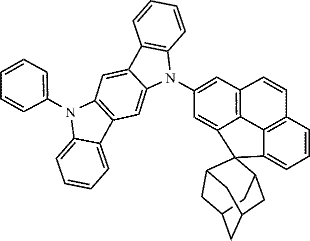 [SMILES: c1ccc(-n2c3ccccc3c3cc4c(cc32)c2ccccc2n4-c2cc3c4c(ccc5cccc(c54)C34C3CC5CC(C3)CC4C5)c2)cc1] 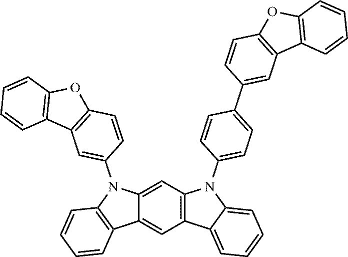 [SMILES: c1ccc2c(c1)oc1ccc(-c3ccc(-n4c5ccccc5c5cc6c7ccccc7n(-c7ccc8oc9ccccc9c8c7)c6cc54)cc3)cc12] 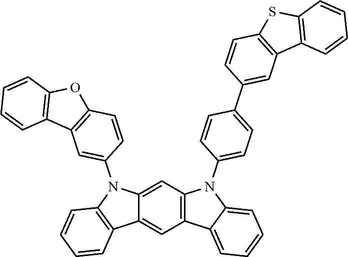 [SMILES: c1ccc2c(c1)oc1ccc(-n3c4ccccc4c4cc5c6ccccc6n(-c6ccc(-c7ccc8sc9ccccc9c8c7)cc6)c5cc43)cc12] 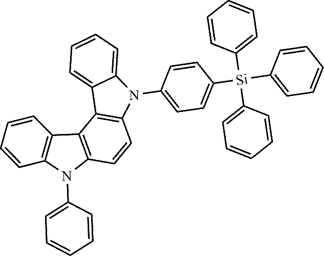 [SMILES: c1ccc(-n2c3ccccc3c3c4c5ccccc5n(-c5ccc([Si](c6ccccc6)(c6ccccc6)c6ccccc6)cc5)c4ccc32)cc1] 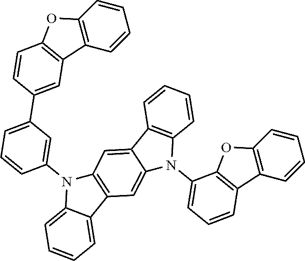 [SMILES: c1cc(-c2ccc3oc4ccccc4c3c2)cc(-n2c3ccccc3c3cc4c(cc32)c2ccccc2n4-c2cccc3c2oc2ccccc23)c1] 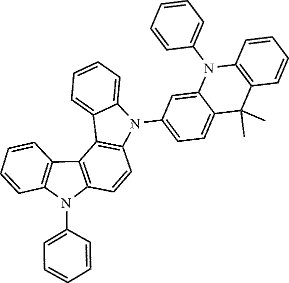 [SMILES: CC1(C)c2ccccc2N(c2ccccc2)c2cc(-n3c4ccccc4c4c5c6ccccc6n(-c6ccccc6)c5ccc43)ccc21] 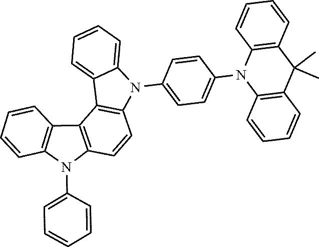 [SMILES: CC1(C)c2ccccc2N(c2ccc(-n3c4ccccc4c4c5c6ccccc6n(-c6ccccc6)c5ccc43)cc2)c2ccccc21] 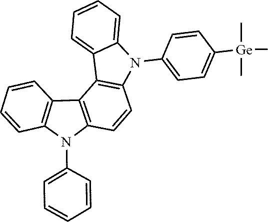 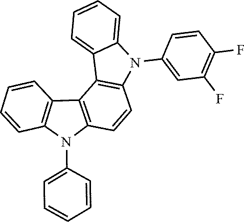 [SMILES: Fc1ccc(-n2c3ccccc3c3c4c5ccccc5n(-c5ccccc5)c4ccc32)cc1F] 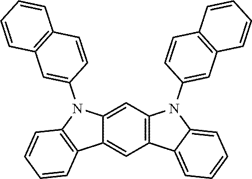 [SMILES: c1ccc2cc(-n3c4ccccc4c4cc5c6ccccc6n(-c6ccc7ccccc7c6)c5cc43)ccc2c1] 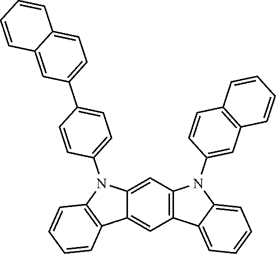 [SMILES: c1ccc2cc(-c3ccc(-n4c5ccccc5c5cc6c7ccccc7n(-c7ccc8ccccc8c7)c6cc54)cc3)ccc2c1] 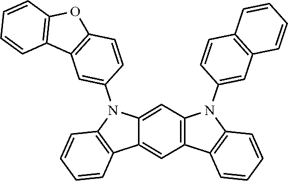 [SMILES: c1ccc2cc(-n3c4ccccc4c4cc5c6ccccc6n(-c6ccc7oc8ccccc8c7c6)c5cc43)ccc2c1] 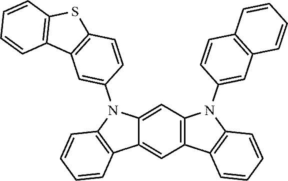 [SMILES: c1ccc2cc(-n3c4ccccc4c4cc5c6ccccc6n(-c6ccc7sc8ccccc8c7c6)c5cc43)ccc2c1] 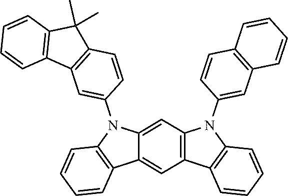 [SMILES: CC1(C)c2ccccc2-c2cc(-n3c4ccccc4c4cc5c6ccccc6n(-c6ccc7ccccc7c6)c5cc43)ccc21] 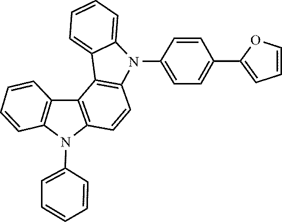 [SMILES: c1ccc(-n2c3ccccc3c3c4c5ccccc5n(-c5ccc(-c6ccco6)cc5)c4ccc32)cc1] 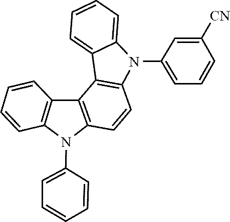 [SMILES: N#Cc1cccc(-n2c3ccccc3c3c4c5ccccc5n(-c5ccccc5)c4ccc32)c1] 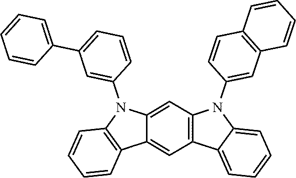 [SMILES: c1ccc(-c2cccc(-n3c4ccccc4c4cc5c6ccccc6n(-c6ccc7ccccc7c6)c5cc43)c2)cc1] 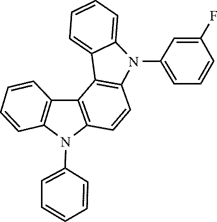 [SMILES: Fc1cccc(-n2c3ccccc3c3c4c5ccccc5n(-c5ccccc5)c4ccc32)c1] 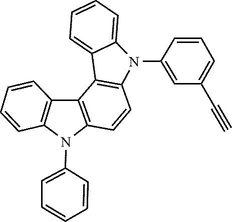 [SMILES: C#Cc1cccc(-n2c3ccccc3c3c4c5ccccc5n(-c5ccccc5)c4ccc32)c1] 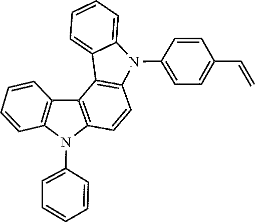 [SMILES: C=Cc1ccc(-n2c3ccccc3c3c4c5ccccc5n(-c5ccccc5)c4ccc32)cc1] 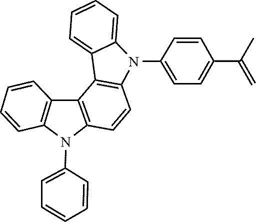 [SMILES: C=C(C)c1ccc(-n2c3ccccc3c3c4c5ccccc5n(-c5ccccc5)c4ccc32)cc1] 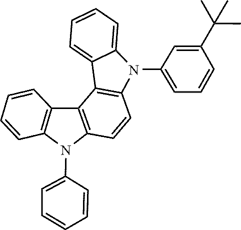 [SMILES: CC(C)(C)c1cccc(-n2c3ccccc3c3c4c5ccccc5n(-c5ccccc5)c4ccc32)c1] 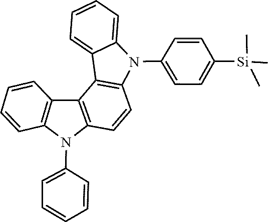 [SMILES: C[Si](C)(C)c1ccc(-n2c3ccccc3c3c4c5ccccc5n(-c5ccccc5)c4ccc32)cc1] 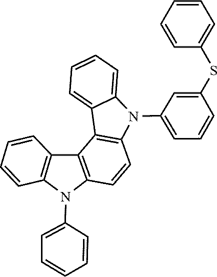 [SMILES: c1ccc(Sc2cccc(-n3c4ccccc4c4c5c6ccccc6n(-c6ccccc6)c5ccc43)c2)cc1] 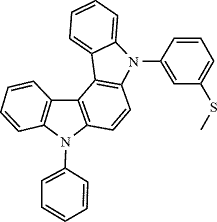 [SMILES: CSc1cccc(-n2c3ccccc3c3c4c5ccccc5n(-c5ccccc5)c4ccc32)c1] 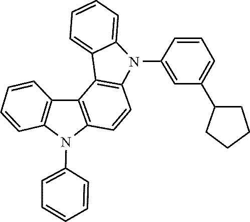 [SMILES: c1ccc(-n2c3ccccc3c3c4c5ccccc5n(-c5cccc(C6CCCC6)c5)c4ccc32)cc1] 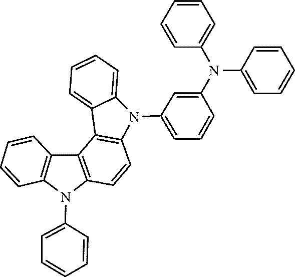 [SMILES: c1ccc(N(c2ccccc2)c2cccc(-n3c4ccccc4c4c5c6ccccc6n(-c6ccccc6)c5ccc43)c2)cc1] 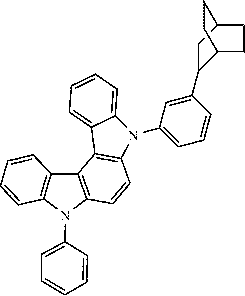 [SMILES: c1ccc(-n2c3ccccc3c3c4c5ccccc5n(-c5cccc(C6CC7CCC6CC7)c5)c4ccc32)cc1] 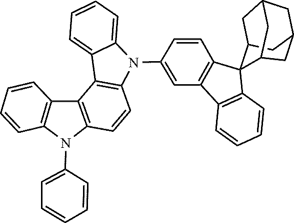 [SMILES: c1ccc(-n2c3ccccc3c3c4c5ccccc5n(-c5ccc6c(c5)-c5ccccc5C65C6CC7CC(C6)CC5C7)c4ccc32)cc1] 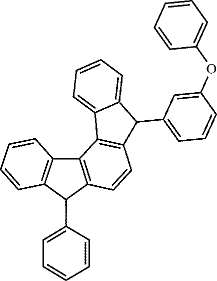 [SMILES: c1ccc(Oc2cccc(C3c4ccccc4-c4c3ccc3c4-c4ccccc4C3c3ccccc3)c2)cc1] 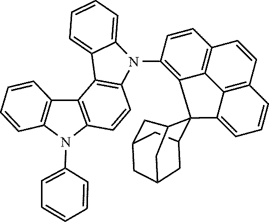 [SMILES: c1ccc(-n2c3ccccc3c3c4c5ccccc5n(-c5ccc6ccc7cccc8c7c6c5C85C6CC7CC(C6)CC5C7)c4ccc32)cc1] 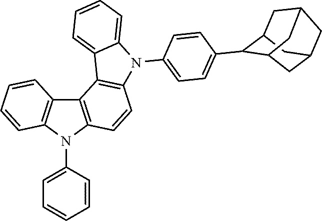 [SMILES: c1ccc(-n2c3ccccc3c3c4c5ccccc5n(-c5ccc(C6C7CC8CC(C7)CC6C8)cc5)c4ccc32)cc1] 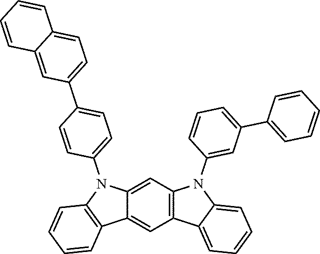 [SMILES: c1ccc(-c2cccc(-n3c4ccccc4c4cc5c6ccccc6n(-c6ccc(-c7ccc8ccccc8c7)cc6)c5cc43)c2)cc1] 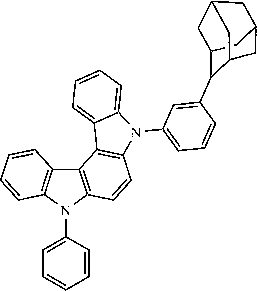 [SMILES: c1ccc(-n2c3ccccc3c3c4c5ccccc5n(-c5cccc(C6C7CC8CC(C7)CC6C8)c5)c4ccc32)cc1] 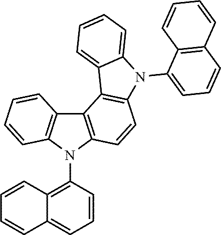 [SMILES: c1ccc2c(-n3c4ccccc4c4c5c6ccccc6n(-c6cccc7ccccc67)c5ccc43)cccc2c1] 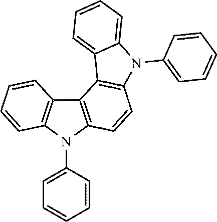 [SMILES: c1ccc(-n2c3ccccc3c3c4c5ccccc5n(-c5ccccc5)c4ccc32)cc1] 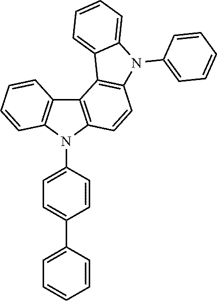 [SMILES: c1ccc(-c2ccc(-n3c4ccccc4c4c5c6ccccc6n(-c6ccccc6)c5ccc43)cc2)cc1] 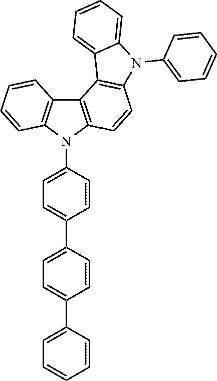 [SMILES: c1ccc(-c2ccc(-c3ccc(-n4c5ccccc5c5c6c7ccccc7n(-c7ccccc7)c6ccc54)cc3)cc2)cc1] 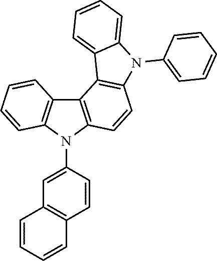 [SMILES: c1ccc(-n2c3ccccc3c3c4c5ccccc5n(-c5ccc6ccccc6c5)c4ccc32)cc1] 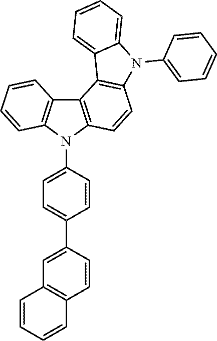 [SMILES: c1ccc(-n2c3ccccc3c3c4c5ccccc5n(-c5ccc(-c6ccc7ccccc7c6)cc5)c4ccc32)cc1] 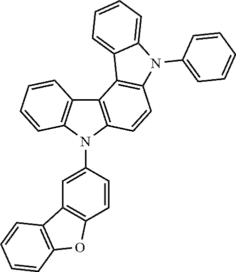 [SMILES: c1ccc(-n2c3ccccc3c3c4c5ccccc5n(-c5ccc6oc7ccccc7c6c5)c4ccc32)cc1] 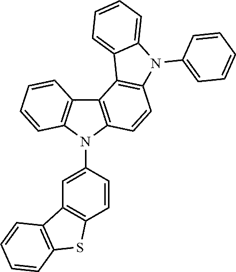 [SMILES: c1ccc(-n2c3ccccc3c3c4c5ccccc5n(-c5ccc6sc7ccccc7c6c5)c4ccc32)cc1] 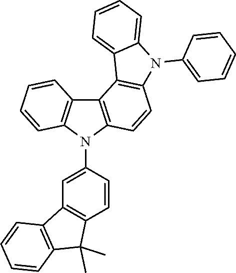 [SMILES: CC1(C)c2ccccc2-c2cc(-n3c4ccccc4c4c5c6ccccc6n(-c6ccccc6)c5ccc43)ccc21] 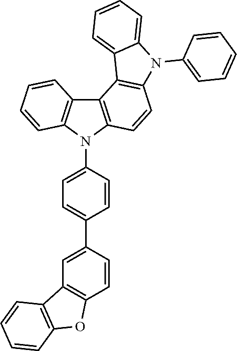 [SMILES: c1ccc(-n2c3ccccc3c3c4c5ccccc5n(-c5ccc(-c6ccc7oc8ccccc8c7c6)cc5)c4ccc32)cc1] 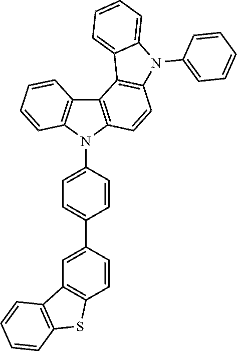 [SMILES: c1ccc(-n2c3ccccc3c3c4c5ccccc5n(-c5ccc(-c6ccc7sc8ccccc8c7c6)cc5)c4ccc32)cc1] 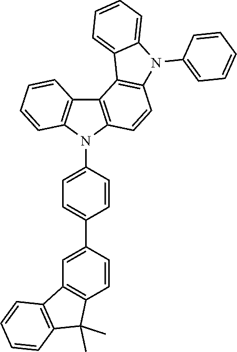 [SMILES: CC1(C)c2ccccc2-c2cc(-c3ccc(-n4c5ccccc5c5c6c7ccccc7n(-c7ccccc7)c6ccc54)cc3)ccc21] 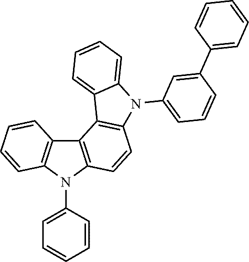 [SMILES: c1ccc(-c2cccc(-n3c4ccccc4c4c5c6ccccc6n(-c6ccccc6)c5ccc43)c2)cc1] 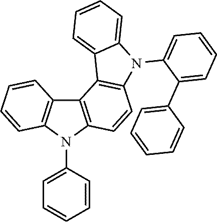 [SMILES: c1ccc(-c2ccccc2-n2c3ccccc3c3c4c5ccccc5n(-c5ccccc5)c4ccc32)cc1] 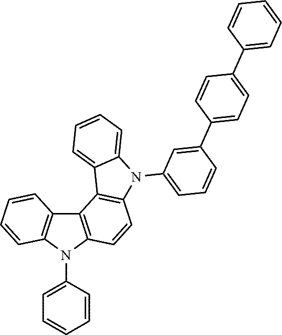 [SMILES: c1ccc(-c2ccc(-c3cccc(-n4c5ccccc5c5c6c7ccccc7n(-c7ccccc7)c6ccc54)c3)cc2)cc1] 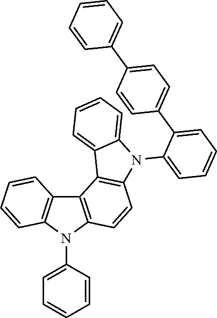 [SMILES: c1ccc(-c2ccc(-c3ccccc3-n3c4ccccc4c4c5c6ccccc6n(-c6ccccc6)c5ccc43)cc2)cc1] 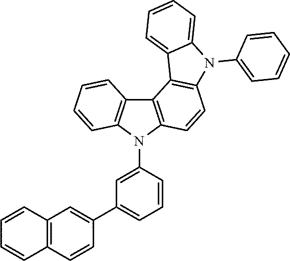 [SMILES: c1ccc(-n2c3ccccc3c3c4c5ccccc5n(-c5cccc(-c6ccc7ccccc7c6)c5)c4ccc32)cc1] 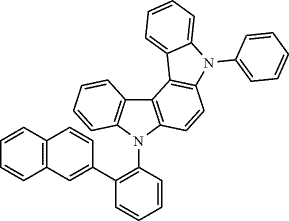 [SMILES: c1ccc(-n2c3ccccc3c3c4c5ccccc5n(-c5ccccc5-c5ccc6ccccc6c5)c4ccc32)cc1] 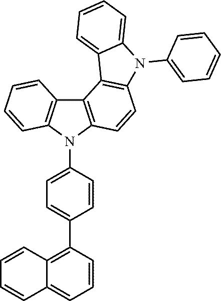 [SMILES: c1ccc(-n2c3ccccc3c3c4c5ccccc5n(-c5ccc(-c6cccc7ccccc67)cc5)c4ccc32)cc1] 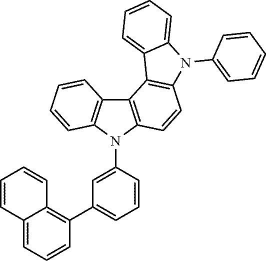 [SMILES: c1ccc(-n2c3ccccc3c3c4c5ccccc5n(-c5cccc(-c6cccc7ccccc67)c5)c4ccc32)cc1] 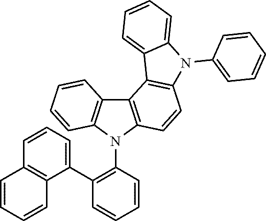 [SMILES: c1ccc(-n2c3ccccc3c3c4c5ccccc5n(-c5ccccc5-c5cccc6ccccc56)c4ccc32)cc1] 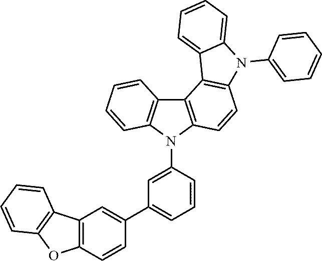 [SMILES: c1ccc(-n2c3ccccc3c3c4c5ccccc5n(-c5cccc(-c6ccc7oc8ccccc8c7c6)c5)c4ccc32)cc1] 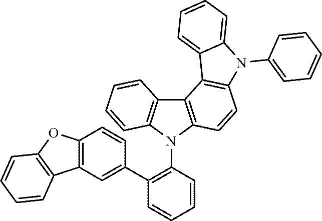 [SMILES: c1ccc(-n2c3ccccc3c3c4c5ccccc5n(-c5ccccc5-c5ccc6oc7ccccc7c6c5)c4ccc32)cc1] 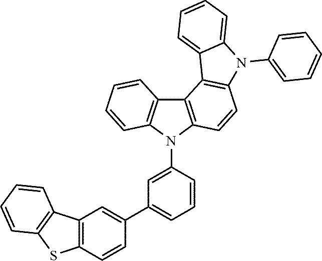 [SMILES: c1ccc(-n2c3ccccc3c3c4c5ccccc5n(-c5cccc(-c6ccc7sc8ccccc8c7c6)c5)c4ccc32)cc1] 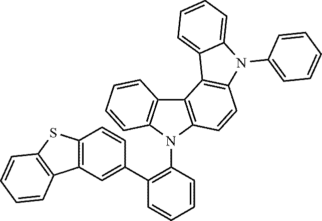 [SMILES: c1ccc(-n2c3ccccc3c3c4c5ccccc5n(-c5ccccc5-c5ccc6sc7ccccc7c6c5)c4ccc32)cc1] 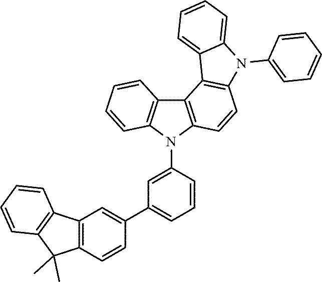 [SMILES: CC1(C)c2ccccc2-c2cc(-c3cccc(-n4c5ccccc5c5c6c7ccccc7n(-c7ccccc7)c6ccc54)c3)ccc21] 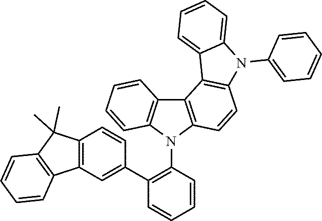 [SMILES: CC1(C)c2ccccc2-c2cc(-c3ccccc3-n3c4ccccc4c4c5c6ccccc6n(-c6ccccc6)c5ccc43)ccc21] 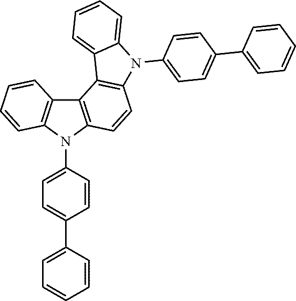 [SMILES: c1ccc(-c2ccc(-n3c4ccccc4c4c5c6ccccc6n(-c6ccc(-c7ccccc7)cc6)c5ccc43)cc2)cc1] 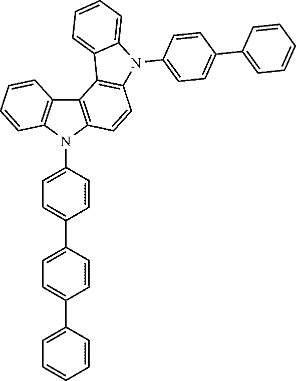 [SMILES: c1ccc(-c2ccc(-c3ccc(-n4c5ccccc5c5c6c7ccccc7n(-c7ccc(-c8ccccc8)cc7)c6ccc54)cc3)cc2)cc1] 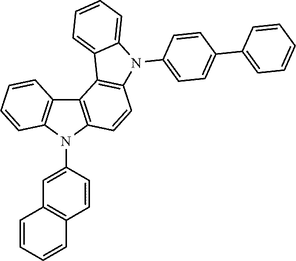 [SMILES: c1ccc(-c2ccc(-n3c4ccccc4c4c5c6ccccc6n(-c6ccc7ccccc7c6)c5ccc43)cc2)cc1] 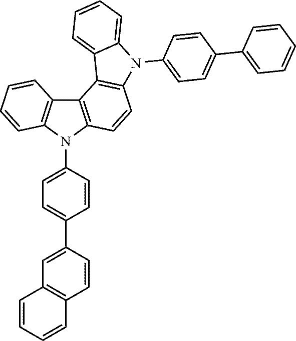 [SMILES: c1ccc(-c2ccc(-n3c4ccccc4c4c5c6ccccc6n(-c6ccc(-c7ccc8ccccc8c7)cc6)c5ccc43)cc2)cc1] 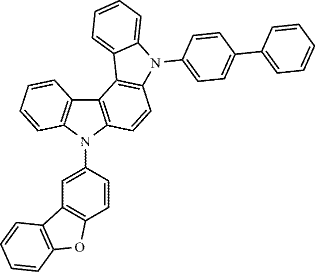 [SMILES: c1ccc(-c2ccc(-n3c4ccccc4c4c5c6ccccc6n(-c6ccc7oc8ccccc8c7c6)c5ccc43)cc2)cc1] 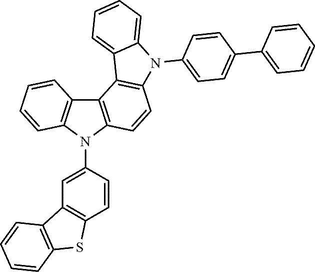 [SMILES: c1ccc(-c2ccc(-n3c4ccccc4c4c5c6ccccc6n(-c6ccc7sc8ccccc8c7c6)c5ccc43)cc2)cc1] 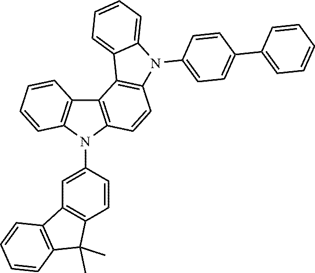 [SMILES: CC1(C)c2ccccc2-c2cc(-n3c4ccccc4c4c5c6ccccc6n(-c6ccc(-c7ccccc7)cc6)c5ccc43)ccc21] 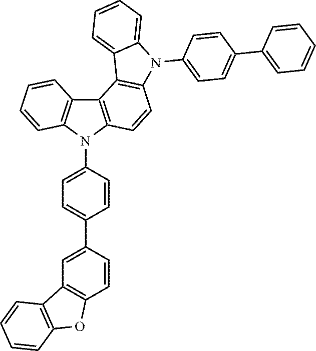 [SMILES: c1ccc(-c2ccc(-n3c4ccccc4c4c5c6ccccc6n(-c6ccc(-c7ccc8oc9ccccc9c8c7)cc6)c5ccc43)cc2)cc1] 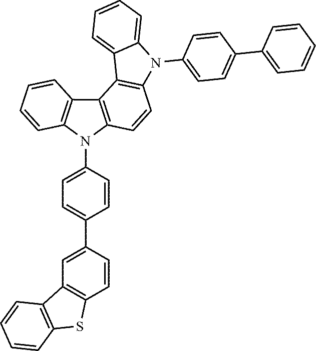 [SMILES: c1ccc(-c2ccc(-n3c4ccccc4c4c5c6ccccc6n(-c6ccc(-c7ccc8sc9ccccc9c8c7)cc6)c5ccc43)cc2)cc1] 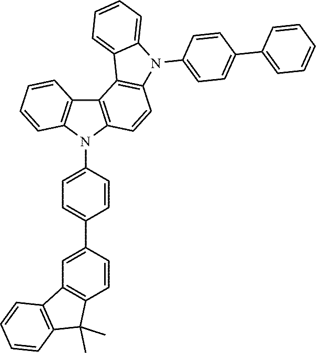 [SMILES: CC1(C)c2ccccc2-c2cc(-c3ccc(-n4c5ccccc5c5c6c7ccccc7n(-c7ccc(-c8ccccc8)cc7)c6ccc54)cc3)ccc21] 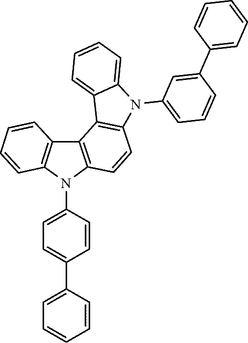 [SMILES: c1ccc(-c2ccc(-n3c4ccccc4c4c5c6ccccc6n(-c6cccc(-c7ccccc7)c6)c5ccc43)cc2)cc1] 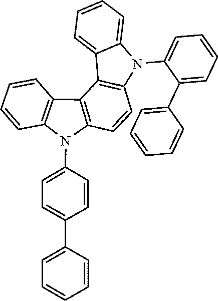 [SMILES: c1ccc(-c2ccc(-n3c4ccccc4c4c5c6ccccc6n(-c6ccccc6-c6ccccc6)c5ccc43)cc2)cc1] 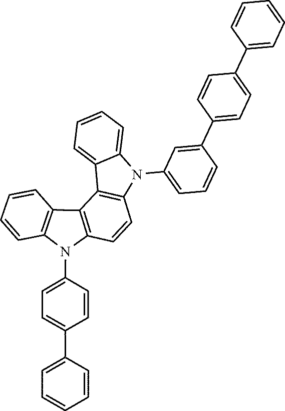 [SMILES: c1ccc(-c2ccc(-c3cccc(-n4c5ccccc5c5c6c7ccccc7n(-c7ccc(-c8ccccc8)cc7)c6ccc54)c3)cc2)cc1] 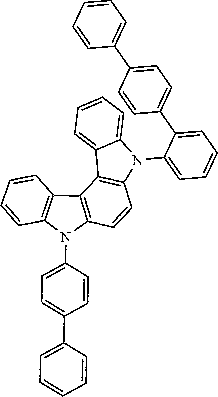 [SMILES: c1ccc(-c2ccc(-c3ccccc3-n3c4ccccc4c4c5c6ccccc6n(-c6ccc(-c7ccccc7)cc6)c5ccc43)cc2)cc1] 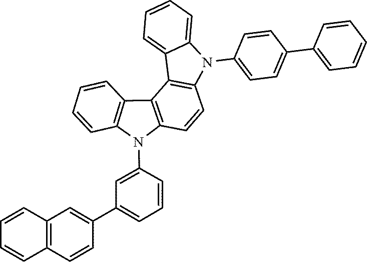 [SMILES: c1ccc(-c2ccc(-n3c4ccccc4c4c5c6ccccc6n(-c6cccc(-c7ccc8ccccc8c7)c6)c5ccc43)cc2)cc1] 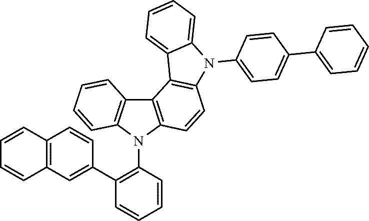 [SMILES: c1ccc(-c2ccc(-n3c4ccccc4c4c5c6ccccc6n(-c6ccccc6-c6ccc7ccccc7c6)c5ccc43)cc2)cc1] 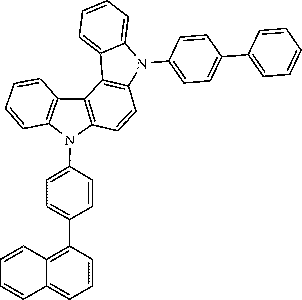 [SMILES: c1ccc(-c2ccc(-n3c4ccccc4c4c5c6ccccc6n(-c6ccc(-c7cccc8ccccc78)cc6)c5ccc43)cc2)cc1] 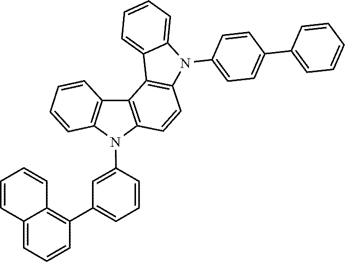 [SMILES: c1ccc(-c2ccc(-n3c4ccccc4c4c5c6ccccc6n(-c6cccc(-c7cccc8ccccc78)c6)c5ccc43)cc2)cc1] 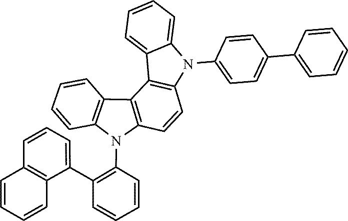 [SMILES: c1ccc(-c2ccc(-n3c4ccccc4c4c5c6ccccc6n(-c6ccccc6-c6cccc7ccccc67)c5ccc43)cc2)cc1] 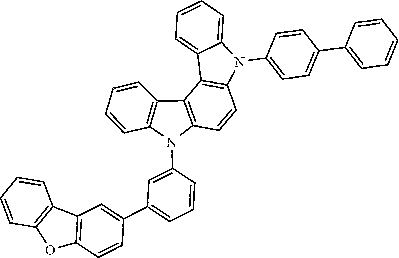 [SMILES: c1ccc(-c2ccc(-n3c4ccccc4c4c5c6ccccc6n(-c6cccc(-c7ccc8oc9ccccc9c8c7)c6)c5ccc43)cc2)cc1] 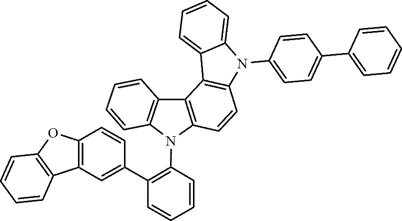 [SMILES: c1ccc(-c2ccc(-n3c4ccccc4c4c5c6ccccc6n(-c6ccccc6-c6ccc7oc8ccccc8c7c6)c5ccc43)cc2)cc1] 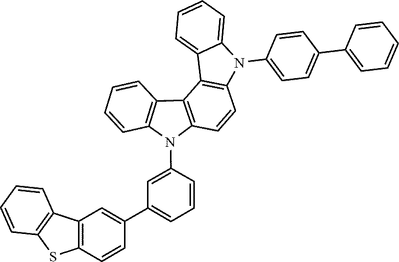 [SMILES: c1ccc(-c2ccc(-n3c4ccccc4c4c5c6ccccc6n(-c6cccc(-c7ccc8sc9ccccc9c8c7)c6)c5ccc43)cc2)cc1] 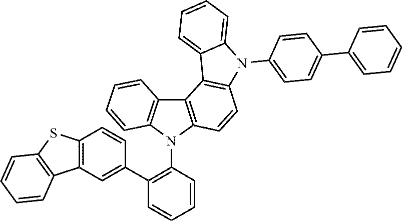 [SMILES: c1ccc(-c2ccc(-n3c4ccccc4c4c5c6ccccc6n(-c6ccccc6-c6ccc7sc8ccccc8c7c6)c5ccc43)cc2)cc1] 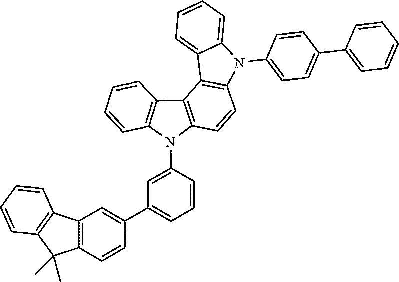 [SMILES: CC1(C)c2ccccc2-c2cc(-c3cccc(-n4c5ccccc5c5c6c7ccccc7n(-c7ccc(-c8ccccc8)cc7)c6ccc54)c3)ccc21] 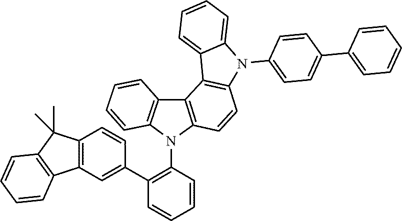 [SMILES: CC1(C)c2ccccc2-c2cc(-c3ccccc3-n3c4ccccc4c4c5c6ccccc6n(-c6ccc(-c7ccccc7)cc6)c5ccc43)ccc21] 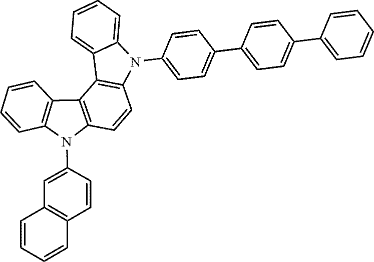 [SMILES: c1ccc(-c2ccc(-c3ccc(-n4c5ccccc5c5c6c7ccccc7n(-c7ccc8ccccc8c7)c6ccc54)cc3)cc2)cc1] 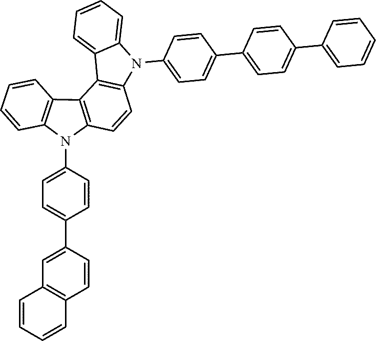 [SMILES: c1ccc(-c2ccc(-c3ccc(-n4c5ccccc5c5c6c7ccccc7n(-c7ccc(-c8ccc9ccccc9c8)cc7)c6ccc54)cc3)cc2)cc1] 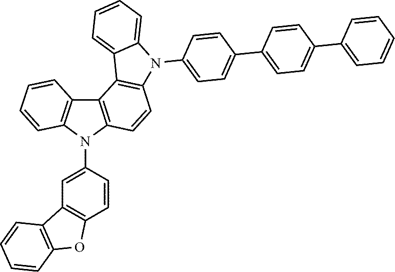 [SMILES: c1ccc(-c2ccc(-c3ccc(-n4c5ccccc5c5c6c7ccccc7n(-c7ccc8oc9ccccc9c8c7)c6ccc54)cc3)cc2)cc1] 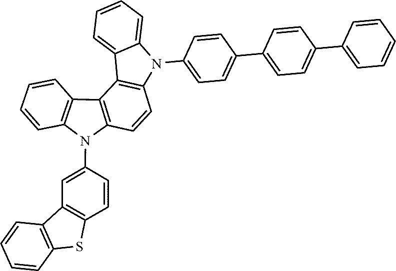 [SMILES: c1ccc(-c2ccc(-c3ccc(-n4c5ccccc5c5c6c7ccccc7n(-c7ccc8sc9ccccc9c8c7)c6ccc54)cc3)cc2)cc1] 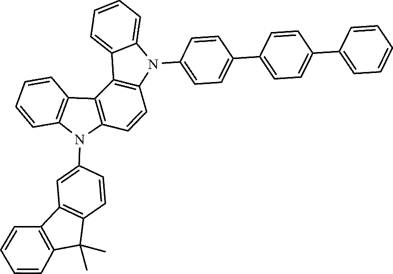 [SMILES: CC1(C)c2ccccc2-c2cc(-n3c4ccccc4c4c5c6ccccc6n(-c6ccc(-c7ccc(-c8ccccc8)cc7)cc6)c5ccc43)ccc21] 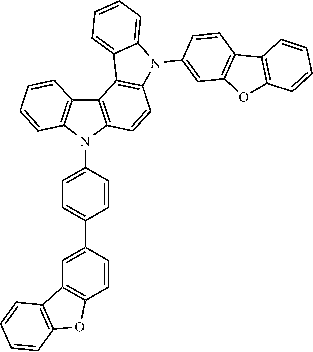 [SMILES: c1ccc2c(c1)oc1cc(-n3c4ccccc4c4c5c6ccccc6n(-c6ccc(-c7ccc8oc9ccccc9c8c7)cc6)c5ccc43)ccc12] 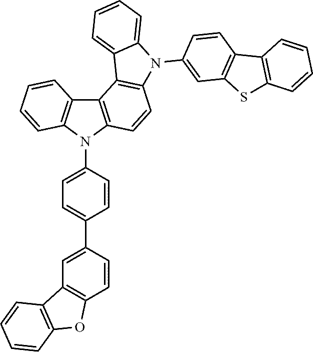 [SMILES: c1ccc2c(c1)oc1ccc(-c3ccc(-n4c5ccccc5c5c6c7ccccc7n(-c7ccc8c(c7)sc7ccccc78)c6ccc54)cc3)cc12] 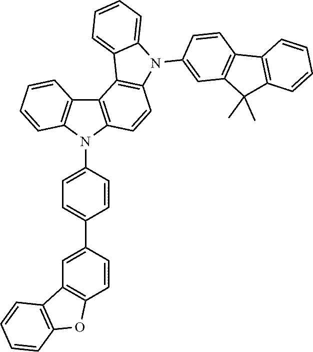 [SMILES: CC1(C)c2ccccc2-c2ccc(-n3c4ccccc4c4c5c6ccccc6n(-c6ccc(-c7ccc8oc9ccccc9c8c7)cc6)c5ccc43)cc21] 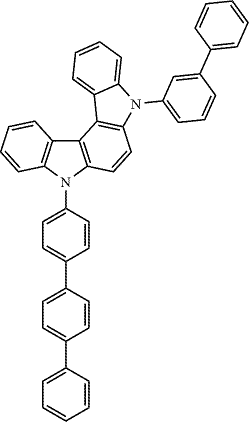 [SMILES: c1ccc(-c2ccc(-c3ccc(-n4c5ccccc5c5c6c7ccccc7n(-c7cccc(-c8ccccc8)c7)c6ccc54)cc3)cc2)cc1] 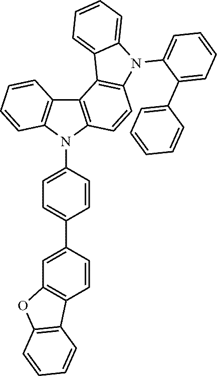 [SMILES: c1ccc(-c2ccccc2-n2c3ccccc3c3c4c5ccccc5n(-c5ccc(-c6ccc7c(c6)oc6ccccc67)cc5)c4ccc32)cc1] 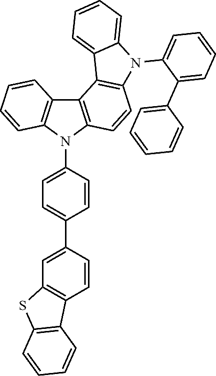 [SMILES: c1ccc(-c2ccccc2-n2c3ccccc3c3c4c5ccccc5n(-c5ccc(-c6ccc7c(c6)sc6ccccc67)cc5)c4ccc32)cc1] 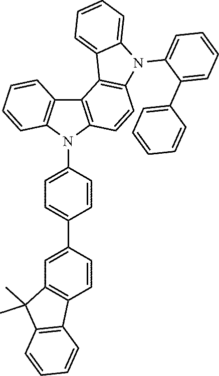 [SMILES: CC1(C)c2ccccc2-c2ccc(-c3ccc(-n4c5ccccc5c5c6c7ccccc7n(-c7ccccc7-c7ccccc7)c6ccc54)cc3)cc21] 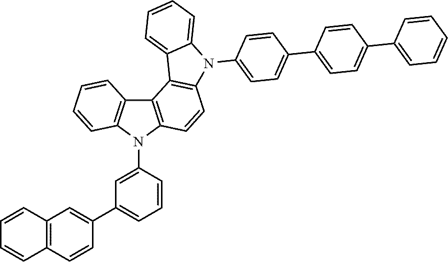 [SMILES: c1ccc(-c2ccc(-c3ccc(-n4c5ccccc5c5c6c7ccccc7n(-c7cccc(-c8ccc9ccccc9c8)c7)c6ccc54)cc3)cc2)cc1] 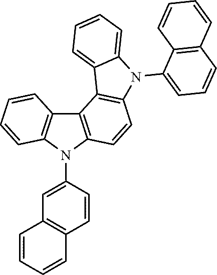 [SMILES: c1ccc2cc(-n3c4ccccc4c4c5c6ccccc6n(-c6cccc7ccccc67)c5ccc43)ccc2c1] 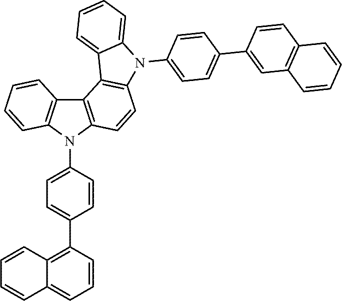 [SMILES: c1ccc2cc(-c3ccc(-n4c5ccccc5c5c6c7ccccc7n(-c7ccc(-c8cccc9ccccc89)cc7)c6ccc54)cc3)ccc2c1] 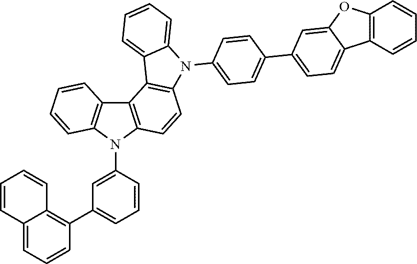 [SMILES: c1cc(-c2cccc3ccccc23)cc(-n2c3ccccc3c3c4c5ccccc5n(-c5ccc(-c6ccc7c(c6)oc6ccccc67)cc5)c4ccc32)c1] 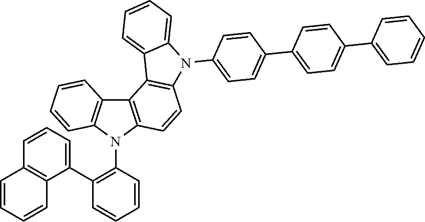 [SMILES: c1ccc(-c2ccc(-c3ccc(-n4c5ccccc5c5c6c7ccccc7n(-c7ccccc7-c7cccc8ccccc78)c6ccc54)cc3)cc2)cc1] 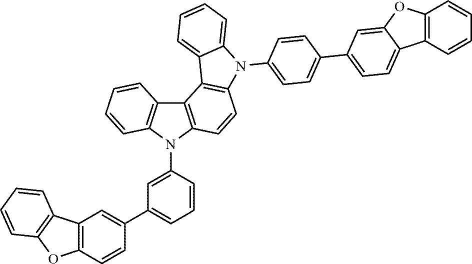 [SMILES: c1cc(-c2ccc3oc4ccccc4c3c2)cc(-n2c3ccccc3c3c4c5ccccc5n(-c5ccc(-c6ccc7c(c6)oc6ccccc67)cc5)c4ccc32)c1] 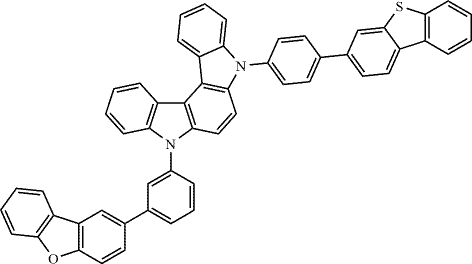 [SMILES: c1cc(-c2ccc3oc4ccccc4c3c2)cc(-n2c3ccccc3c3c4c5ccccc5n(-c5ccc(-c6ccc7c(c6)sc6ccccc67)cc5)c4ccc32)c1] 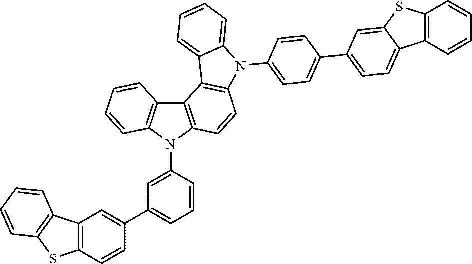 [SMILES: c1cc(-c2ccc3sc4ccccc4c3c2)cc(-n2c3ccccc3c3c4c5ccccc5n(-c5ccc(-c6ccc7c(c6)sc6ccccc67)cc5)c4ccc32)c1] 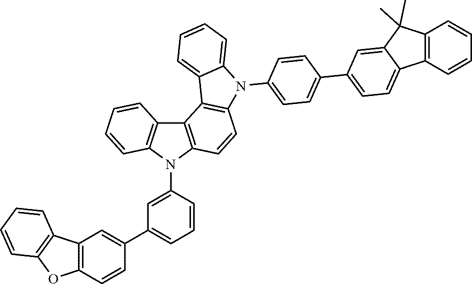 [SMILES: CC1(C)c2ccccc2-c2ccc(-c3ccc(-n4c5ccccc5c5c6c7ccccc7n(-c7cccc(-c8ccc9oc%10ccccc%10c9c8)c7)c6ccc54)cc3)cc21] 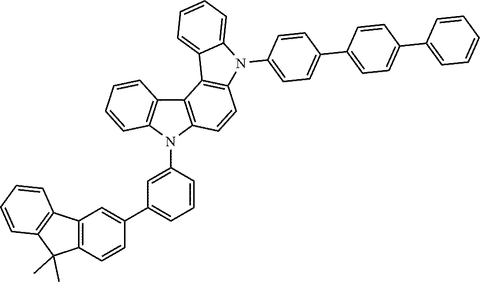 [SMILES: CC1(C)c2ccccc2-c2cc(-c3cccc(-n4c5ccccc5c5c6c7ccccc7n(-c7ccc(-c8ccc(-c9ccccc9)cc8)cc7)c6ccc54)c3)ccc21] 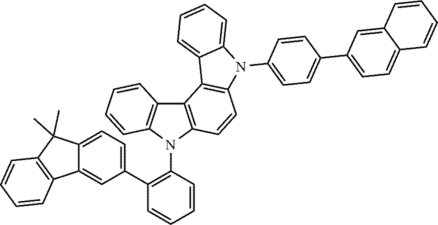 [SMILES: CC1(C)c2ccccc2-c2cc(-c3ccccc3-n3c4ccccc4c4c5c6ccccc6n(-c6ccc(-c7ccc8ccccc8c7)cc6)c5ccc43)ccc21] 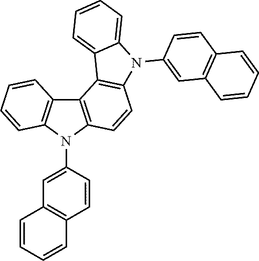 [SMILES: c1ccc2cc(-n3c4ccccc4c4c5c6ccccc6n(-c6ccc7ccccc7c6)c5ccc43)ccc2c1] 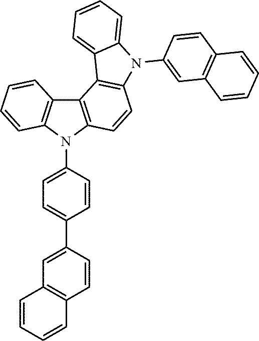 [SMILES: c1ccc2cc(-c3ccc(-n4c5ccccc5c5c6c7ccccc7n(-c7ccc8ccccc8c7)c6ccc54)cc3)ccc2c1] 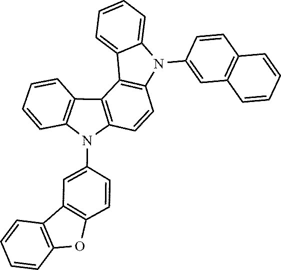 [SMILES: c1ccc2cc(-n3c4ccccc4c4c5c6ccccc6n(-c6ccc7oc8ccccc8c7c6)c5ccc43)ccc2c1] 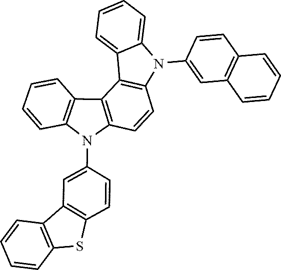 [SMILES: c1ccc2cc(-n3c4ccccc4c4c5c6ccccc6n(-c6ccc7sc8ccccc8c7c6)c5ccc43)ccc2c1] 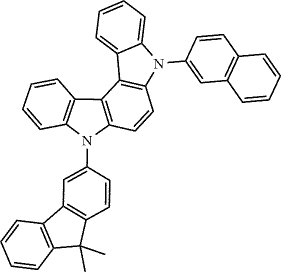 [SMILES: CC1(C)c2ccccc2-c2cc(-n3c4ccccc4c4c5c6ccccc6n(-c6ccc7ccccc7c6)c5ccc43)ccc21] 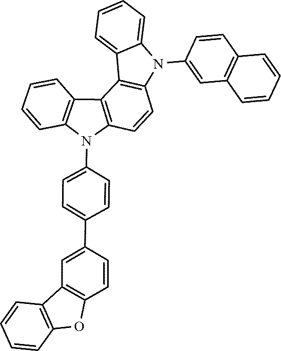 [SMILES: c1ccc2cc(-n3c4ccccc4c4c5c6ccccc6n(-c6ccc(-c7ccc8oc9ccccc9c8c7)cc6)c5ccc43)ccc2c1] 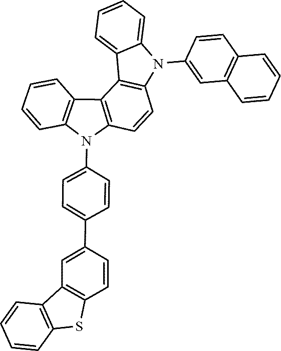 [SMILES: c1ccc2cc(-n3c4ccccc4c4c5c6ccccc6n(-c6ccc(-c7ccc8sc9ccccc9c8c7)cc6)c5ccc43)ccc2c1] 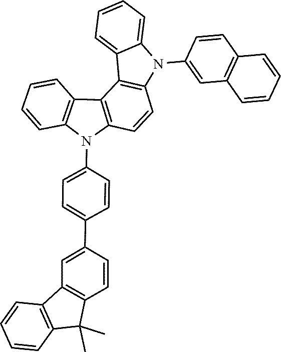 [SMILES: CC1(C)c2ccccc2-c2cc(-c3ccc(-n4c5ccccc5c5c6c7ccccc7n(-c7ccc8ccccc8c7)c6ccc54)cc3)ccc21] 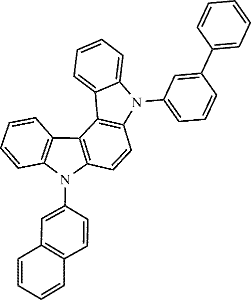 [SMILES: c1ccc(-c2cccc(-n3c4ccccc4c4c5c6ccccc6n(-c6ccc7ccccc7c6)c5ccc43)c2)cc1] 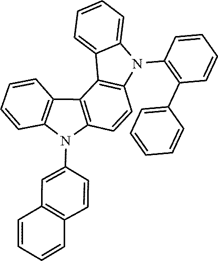 [SMILES: c1ccc(-c2ccccc2-n2c3ccccc3c3c4c5ccccc5n(-c5ccc6ccccc6c5)c4ccc32)cc1] 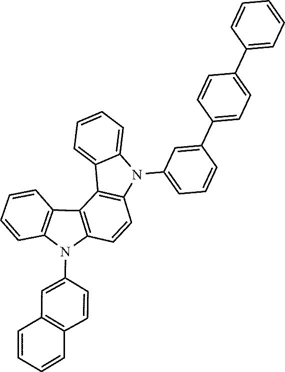 [SMILES: c1ccc(-c2ccc(-c3cccc(-n4c5ccccc5c5c6c7ccccc7n(-c7ccc8ccccc8c7)c6ccc54)c3)cc2)cc1] 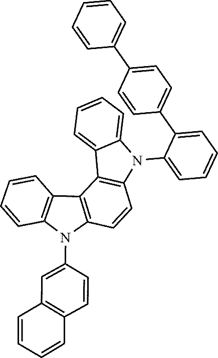 [SMILES: c1ccc(-c2ccc(-c3ccccc3-n3c4ccccc4c4c5c6ccccc6n(-c6ccc7ccccc7c6)c5ccc43)cc2)cc1] 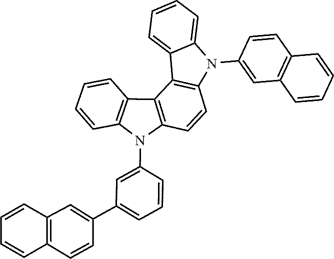 [SMILES: c1cc(-c2ccc3ccccc3c2)cc(-n2c3ccccc3c3c4c5ccccc5n(-c5ccc6ccccc6c5)c4ccc32)c1] 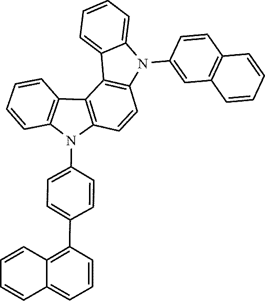 [SMILES: c1ccc2cc(-n3c4ccccc4c4c5c6ccccc6n(-c6ccc(-c7cccc8ccccc78)cc6)c5ccc43)ccc2c1] 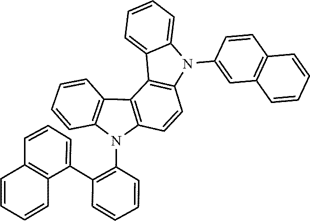 [SMILES: c1ccc(-n2c3ccccc3c3c4c5ccccc5n(-c5ccc6ccccc6c5)c4ccc32)c(-c2cccc3ccccc23)c1] 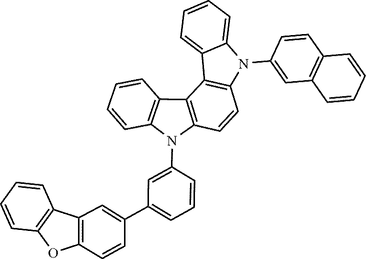 [SMILES: c1cc(-c2ccc3oc4ccccc4c3c2)cc(-n2c3ccccc3c3c4c5ccccc5n(-c5ccc6ccccc6c5)c4ccc32)c1] 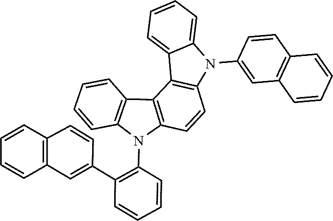 [SMILES: c1ccc(-n2c3ccccc3c3c4c5ccccc5n(-c5ccc6ccccc6c5)c4ccc32)c(-c2ccc3ccccc3c2)c1] 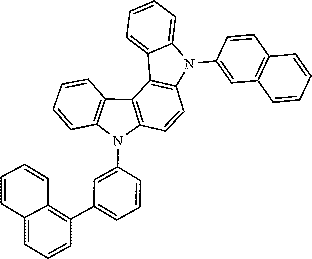 [SMILES: c1cc(-c2cccc3ccccc23)cc(-n2c3ccccc3c3c4c5ccccc5n(-c5ccc6ccccc6c5)c4ccc32)c1] 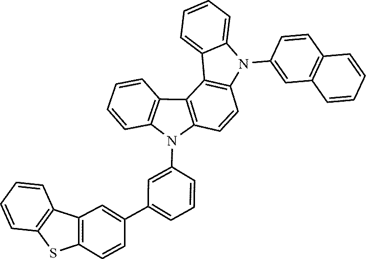 [SMILES: c1cc(-c2ccc3sc4ccccc4c3c2)cc(-n2c3ccccc3c3c4c5ccccc5n(-c5ccc6ccccc6c5)c4ccc32)c1] 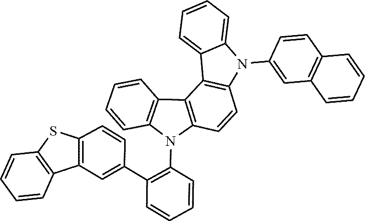 [SMILES: c1ccc(-n2c3ccccc3c3c4c5ccccc5n(-c5ccc6ccccc6c5)c4ccc32)c(-c2ccc3sc4ccccc4c3c2)c1] 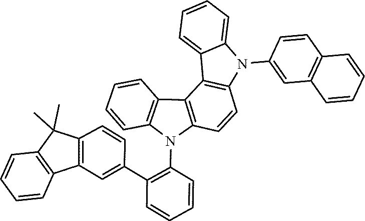 [SMILES: CC1(C)c2ccccc2-c2cc(-c3ccccc3-n3c4ccccc4c4c5c6ccccc6n(-c6ccc7ccccc7c6)c5ccc43)ccc21] 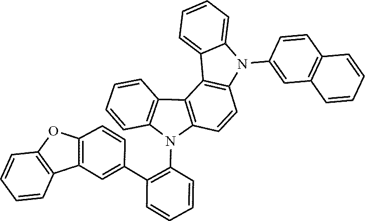 [SMILES: c1ccc(-n2c3ccccc3c3c4c5ccccc5n(-c5ccc6ccccc6c5)c4ccc32)c(-c2ccc3oc4ccccc4c3c2)c1] 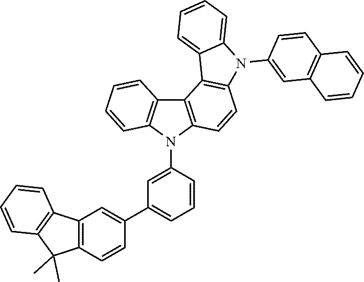 [SMILES: CC1(C)c2ccccc2-c2cc(-c3cccc(-n4c5ccccc5c5c6c7ccccc7n(-c7ccc8ccccc8c7)c6ccc54)c3)ccc21] 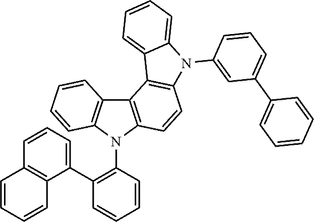 [SMILES: c1ccc(-c2cccc(-n3c4ccccc4c4c5c6ccccc6n(-c6ccccc6-c6cccc7ccccc67)c5ccc43)c2)cc1] 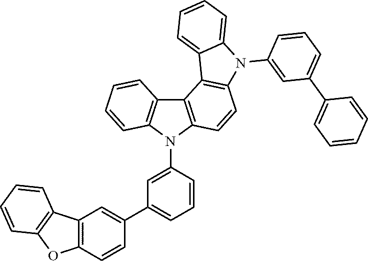 [SMILES: c1ccc(-c2cccc(-n3c4ccccc4c4c5c6ccccc6n(-c6cccc(-c7ccc8oc9ccccc9c8c7)c6)c5ccc43)c2)cc1] 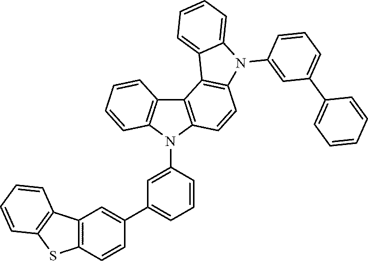 [SMILES: c1ccc(-c2cccc(-n3c4ccccc4c4c5c6ccccc6n(-c6cccc(-c7ccc8sc9ccccc9c8c7)c6)c5ccc43)c2)cc1] 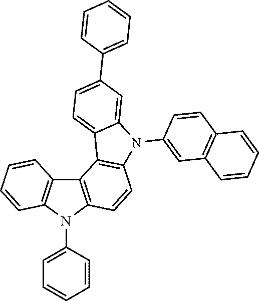 [SMILES: c1ccc(-c2ccc3c4c5c6ccccc6n(-c6ccccc6)c5ccc4n(-c4ccc5ccccc5c4)c3c2)cc1] 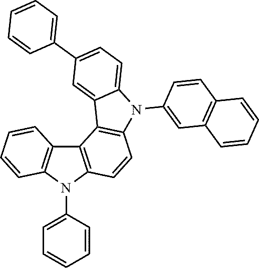 [SMILES: c1ccc(-c2ccc3c(c2)c2c4c5ccccc5n(-c5ccccc5)c4ccc2n3-c2ccc3ccccc3c2)cc1] 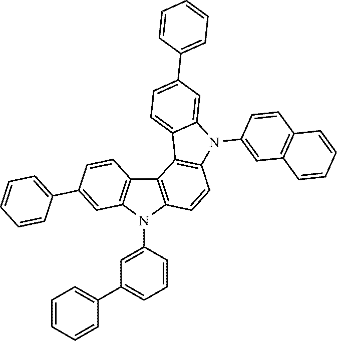 [SMILES: c1ccc(-c2cccc(-n3c4cc(-c5ccccc5)ccc4c4c5c6ccc(-c7ccccc7)cc6n(-c6ccc7ccccc7c6)c5ccc43)c2)cc1] 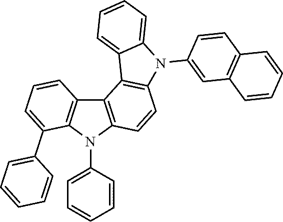 [SMILES: c1ccc(-c2cccc3c4c5c6ccccc6n(-c6ccc7ccccc7c6)c5ccc4n(-c4ccccc4)c23)cc1] 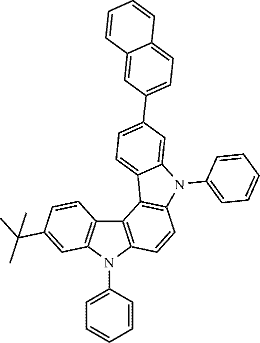 [SMILES: CC(C)(C)c1ccc2c3c4c5ccc(-c6ccc7ccccc7c6)cc5n(-c5ccccc5)c4ccc3n(-c3ccccc3)c2c1]
wherein optionally, hydrogens in Compound 2-1 to Compound 2-305 can be partially or fully substituted with deuterium.

12. The electroluminescent device according to claim 1, wherein the organic layer is a light-emitting layer, and the first compound and the second compound are host materials.

13. The electroluminescent device according to claim 12, wherein the light-emitting layer further comprises at least one phosphorescent material.

14. The electroluminescent device according to claim 13, wherein the at least one phosphorescent material is a metal complex having a general formula of M(Lₐ)ₘ(L_{b})ₙ(L_{c})_{q};
M is selected from a metal with a relative atomic mass greater than 40; and
Lₐ, L_{b} and L_{c} are a first ligand, a second ligand and a third ligand coordinated to M, respectively; Lₐ, L_{b} and L_{c} can be optionally joined to form a multidentate ligand; Lₐ, L_{b} and L_{c} may be identical or different; m is 1, 2 or 3; n is 0, 1 or 2; q is 0, 1 or 2; the sum of m, n and q is equal to an oxidation state of M; when m is greater than or equal to 2, a plurality of Lₐ may be identical or different; when n is 2, two L_{b} may be identical or different; when q is 2, two L_{c} may be identical or different;
wherein Lₐ has a structure represented by Formula 3: 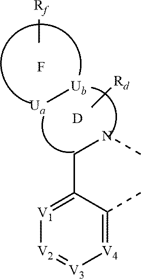
wherein the ring D is selected from a five-membered heteroaromatic ring or a six-membered heteroaromatic ring;
the ring F is selected from a five-membered unsaturated carbocyclic ring, a benzene ring, a five-membered heteroaromatic ring or a six-membered heteroaromatic ring;
the ring D and the ring F are fused via Uₐ and U_{b};
Uₐ and U_{b} are, at each occurrence identically or differently, selected from C or N;
R_{d} and R_{f} represent, at each occurrence identically or differently, mono-substitution, multiple substitutions or non-substitution;
V₁ to V₄ are, at each occurrence identically or differently, selected from CRᵥ or N;
R_{d}, R_{f} and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{d}, R_{f}, Rᵥ can be optionally joined to form a ring;
wherein L_{b} and L_{c} are, at each occurrence identically or differently, selected from any one of the following structures: 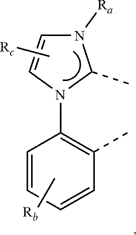 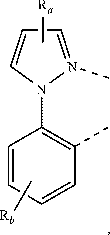 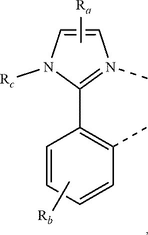 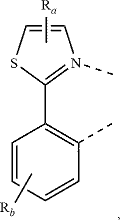 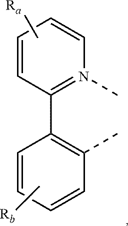 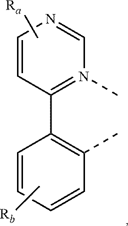 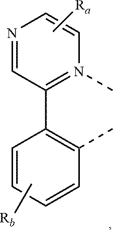 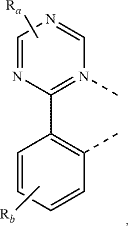 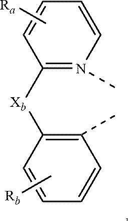 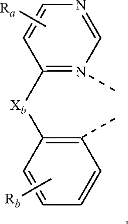 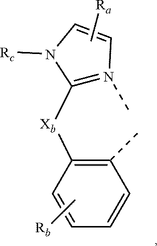 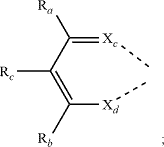
wherein Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
in the structures of the ligands L_{b} and L_{c}, adjacent substituents Rₐ, R_{b}, R_{c}, R_{N1}, R_{N2}, R_{C1} and R_{C2} can be optionally joined to form a ring.

15. The electroluminescent device according to claim 13, wherein the at least one phosphorescent material is a metal complex having a general formula of M(Lₐ)ₘ(L_{b})ₙ;
M is selected from a metal with a relative atomic mass greater than 40; and
Lₐ and L_{b} are a first ligand and a second ligand coordinated to M, respectively; Lₐ and L_{b} can be optionally joined to form a multidentate ligand; m is 1, 2 or 3; n is 0, 1 or 2; the sum of m and n is equal to an oxidation state of M; when m is greater than or equal to 2, a plurality of Lₐ may be identical or different; when n is 2, two L_{b} may be identical or different;
wherein Lₐ has a structure represented by Formula 3: 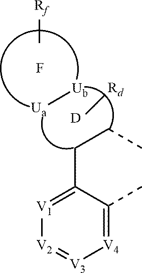
wherein the ring D is selected from a five-membered heteroaromatic ring or a six-membered heteroaromatic ring;
the ring F is selected from a five-membered unsaturated carbocyclic ring, a benzene ring, a five-membered heteroaromatic ring or a six-membered heteroaromatic ring;
the ring D and the ring F are fused via Uₐ and U_{b};
Uₐ and U_{b} are, at each occurrence identically or differently, selected from C or N;
R_{d} and R_{f} represent, at each occurrence identically or differently, mono-substitution, multiple substitutions or non-substitution;
V₁ to V₄ are, at each occurrence identically or differently, selected from CRᵥ or N;
R_{d}, R_{f} and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{d}, R_{f}, Rᵥ can be optionally joined to form a ring;
wherein the ligand L_{b} has the following structure: 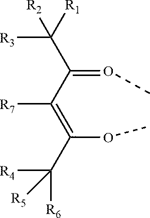
wherein R₁ to R₇ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
preferably, at least one or two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one or two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and
more preferably, at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof; and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

16. An electronic apparatus, comprising the electroluminescent device according to claim 1.

17. A compound combination, comprising a first compound and a second compound, wherein the first compound has a structure of H-L-E, wherein H has a structure represented by Formula 1: 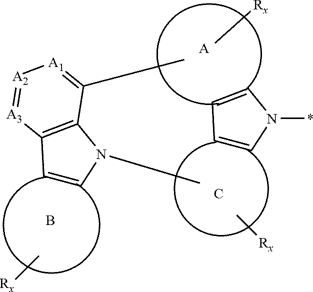
wherein in Formula 1, A₁, A₂ and A₃ are, at each occurrence identically or differently, selected from N or CR, and the ring A, the ring B and the ring C are, at each occurrence identically or differently, selected from a carbocyclic ring having 5 to 18 carbon atoms or a heterocyclic ring having 3 to 18 carbon atoms; and
Rₓ represents, at each occurrence identically or differently, mono-substitution, multiple substitutions or non-substitution;
wherein E has a structure represented by Formula 1-a: 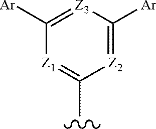
wherein in Formula 1-a, Ar is, at each occurrence identically or differently, selected from substituted or unsubstituted aryl having 6 to 30 carbon atoms or substituted or unsubstituted heteroaryl having 3 to 30 carbon atoms;
Z₁ to Z₃ are each independently selected from N or CR_{z}, and at least one of Z₁ to Z₃ is N;
L is selected from a single bond, substituted or unsubstituted arylene having 6 to 30 carbon atoms, substituted or unsubstituted heteroarylene having 3 to 30 carbon atoms or a combination thereof;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R, Rₓ can be optionally joined to form a ring;
wherein the second compound has a structure represented by Formula 2: 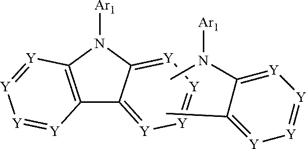
wherein Y is, at each occurrence identically or differently, selected from C, CR_{Y} or N;
R_{Y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and
adjacent substituents R_{Y} can be optionally joined to form a ring.
